(19) United States
(12) Patent Application Publication
(10) Pub. No.: US 2017/0001970 A1
(43) Pub. Date: Jan. 5, 2017

(54) Substituted benzohydrazide analogs as histone demethylase inhibitors

(71) Applicant: University of Utah Research Foundation, Salt Lake City, UT (US)

(72) Inventors: Hariprasad Vankayalapati, Draper, UT (US); Sunil Sharma, Salt Lake City, UT (US); Xiaohui Liu, Salt Lake City, UT (US); Craig Coburn, Salt Lake City, UT (US)

(21) Appl. No.: 15/200,555

(22) Filed: Jul. 1, 2016

Related U.S. Application Data
(60) Provisional application No. 62/188,328, filed on Jul. 2, 2015.

(51) Int. Cl. C07D 295/26; C07D 213/82; C07D 211/96

(52) U.S. Cl. CPC C07D 295/26; C07D 211/96; C07D 213/82

(57) ABSTRACT

Benzohydrazide analogs, derivatives thereof, and related compounds, which are useful as inhibitors of lysine-specific histone demethylase, including LSD1 and LSD2; synthetic methods for making the compounds; pharmaceutical compositions comprising the compounds; and methods of using the compounds and compositions to treat disorders associated with dysfunction of the LSD1 and/or LSD2.

### BACKGROUND

Over the past decade it has become clear that epigenetic changes, which alter gene activity without altering DNA sequence, collaborate with genetic mistakes to promote cancer development and progression (Tsai, H. C. and Baylin, S. B. Cell Res 2011, 21 (3), 502-17; and Fullgrabe, J., Kavanagh, E., and Joseph, B. Oncogene 2011). The regulation of the modifications on DNA and the proteins associated with DNA has become an area of intense interest and the enzymes involved in these processes have been suggested as a new class of protein targets for drug development. The major proteins associated with DNA are histone proteins. Histone tails are subject to a variety of posttranslational modifications, such as phosphorylation, acetylation, methylation, and ubiquitination, and these modifications, especially acetylation and methylation on lysine residues, play a major role in the regulation of gene expression, and are often dysregulated in cancer (Fullgrabe, J., Kavanagh, E., and Joseph, B. Oncogene 2011).

Recently an enzyme called Lysine-Specific Demethylase 1 (LSD1) was found to catalyze the oxidative demethylation of monomethylated and dimethylated histone H3 at lysine 4 (H3K4me1 and H3K4me2) and lysine 9 (H3K9me1 and H3K9me2) through a flavin adenine dinucleotide (FAD)-dependent reaction (Shi, Y., et al. Cell 2004, 119 (7), 941-53; and Metzger, E., et al. Nature 2005, 437 (7057), 436-9), Whereas histone acetylation is associated with loose chromatin and gene activation, methylation of histones is less straightforward. Using the lysine residues regulated by LSD1 as an example, methylation at H3K4 is generally associated with gene activation, while methylation of H3K9 is associated with transcriptional repression.

There is currently one known mammalian homolog of LSD1 which is a protein variously designated LSD2, KDM1b, and AOF1. It shares a similar domain homology, but exhibits less than 31% sequence identity (Fang, R. et al. Molecular Cell 2010, 39:222-233). It has been shown that LSD2 is a H3K4me1/2 demethylase that specifically regulates histone H3K4 methylation within intragenic regions of its target genes (ibid.). Both LSD1 and LSD2 contain a SWIRM domain, a FAD coenzyme-binding motif, and a C-terminal amine oxidase domain, all of which are critical to the enzymatic activity. However, unlike LSD1, the protein LSD2 contains a CW-type zinc finger domain in its N-terminal domain, a region which is unstructured in LSD1. Furthermore, LSD2 lacks the “tower domain” of LSD1. At a cellular level, it has been suggested that LSD2 has a role in transcriptional regulation (ibid.). As expected, LSD2 appears to play a role in regulating DNA methylation as well, although the role in DNA methylation may be developmental stage specific (ibid.; Ciccone, D. N., et al. Nature 2009 461:415-418; Karytinos, A., et al. J. Biol. Chem. 2009 284:17775-17782; and Yang, Z., et al. Cell Res. 2010 20:276-287).

Several lines of evidence point to LSD1 as being a possible therapeutic target in cancer. LSD1 is reportedly over-expressed in a variety of tumors including neuroblastoma, ER-negative breast, bladder, lung, and colorectal tumors (Schulte, J. H., et al. Cancer Res 2009, 69 (5), 2065-71; Lim, S., et al. Carcinogenesis 2010, 31 (3), 512-20; and Hayami, S., et al. Int J Cancer 2011, 128 (3), 574-86). Increased methylation of the permissive H3K4 mark by LSD1 inhibition has been shown to reactivate expression of tumor suppressor genes in cancer models (Huang, Y., et al. Clin Cancer Res 2009, 15 (23), 7217-28). In addition, LSD1 has been found to associate with estrogen and androgen receptors leading to the specific demethylation of the repressive H3K9 mark, thereby increasing target gene expression (Metzger, E., et al. Nature 2005, 437 (7057), 436-9; and Garcia-Bassets, I., et al. Cell 2007, 128 (3), 505-18). Thus, depending upon cofactors bound to LSD1, demethylation by LSD1 can contribute to cancer through both the permissive H3K4 and the repressive H3K9 mark. Therefore, the inhibition of LSD1 might be an effective strategy for re-expression of epigenetically silenced tumor suppressor genes as well as down regulation of important cancer pathways in a number of cancer types. Several LSD1 inhibitors have been reported, but they have shown poor selectivity and/or pharmacological properties, making further exploration of LSD1 biology difficult.

Monoamine oxidase (MAO) inhibitors such as tranylcypromine and pargyline have been reported as LSD1 inhibitors, and there have been several reports regarding attempts to discover derivatives with increased selectivity for LSD1 over MAO (Mimasu, S., et al. Biochemistry 2010, 49 (30), 6494-503; Binda, C., et al. J Am Chem Soc 2010, 132 (19), 6827-33; Culhane, J. C., et al. J Am Chem Soc 2006, 128 (14), 4536-7; Culhane, J. C., et al. J Am Chem Soc 2010, 132 (9), 3164-76; and Ueda, R. , et al. J Am Chem Soc 2009, 131 (48), 17536-7). These compounds irreversibly inactivate LSD1 by covalent binding to the FAD cofactor. Polyamine derivatives have also been evaluated as LSD1 inhibitors, where compounds with activity in the μM range have been described (Huang, Y., et al. Clin Cancer Res 2009, 15 (23), 7217-28; Sharma, S. K., et al. J Med Chem 2010, 53 (14), 5197-212; and Huang, Y., et al. Proc Natl Acad Sci USA 2007, 104 (19), 8023-8). In general, these and other reported LSD1 inhibitors are neither adequately selective nor potent enough to optimally interact with the crucial amino acid residues of the substrate-binding site present in LSD1.

In summary, the LSD proteins play a key role in epigenetic and transcriptional regulation, and they are frequently altered in mammalian cancers, thus making them an attractive target for therapeutic intervention. Despite advances in drug discovery directed to identifying inhibitors of LSD1 and/or LSD2 protein activity, there is still a scarcity of compounds that are both potent, efficacious, and selective inhibitors of either LSD1 or LSD2. Furthermore, there is a scarcity of compounds effective in the treatment of cancer and other diseases associated with dysfunction in LSD1 and/or LSD2. These needs and other needs are satisfied by the present invention.

### SUMMARY

The invention, in one aspect, relates to compounds useful useful as inhibitors of lysine-specific demethylase, or LSD. In a further aspect, the disclosed compounds and products of disclosed methods of making, or a pharmaceutically acceptable salt, hydrate, solvate, or polymorph thereof, are modulators of LSD activity, methods of making same, pharmaceutical compositions comprising same, and methods of treating disorders associated with a LSD activity dysfunction using same. In a still further aspect, the present invention relates to compounds that bind to a LSD protein and negatively modulate LSD activity. The disclosed compounds can, in one aspect, exhibit subtype selectivity. In a further aspect, the disclosed compounds exhibit selectivity for the LSD1 member of the LSD protein family. In a still further aspect, the disclosed compounds exhibit selectivity for the LSD2 member of the LSD protein family.

Also disclosed are pharmaceutical compositions comprising, a therapeutically effective amount of a disclosed compound and a pharmaceutically acceptable carrier.

Also disclosed are synthetic methods for making the disclosed compounds. In a further aspect, disclosed are the products of the disclosed synthetic methods.

Disclosed are methods for the treatment of a disorder associated with a LSD activity dysfunction in a mammal comprising the step of administering to the mammal a therapeutically effective amount of a disclosed compound, or a pharmaceutically acceptable salt, hydrate, solvate, or polymorph thereof.

Also disclosed are methods for inhibition of LSD activity in a mammal comprising the step of administering to the mammal a therapeutically effective amount of least one disclosed compound, or a pharmaceutically acceptable salt, hydrate, solvate, or polymorph thereof.

Also disclosed are methods for inhibiting LSD activity in at least one cell, comprising the step of contacting the at least one cell with an effective amount of least one disclosed compound, or a pharmaceutically acceptable salt, hydrate, solvate, or polymorph thereof.

Also disclosed are uses of a disclosed compound, or a pharmaceutically acceptable salt, hydrate, solvate, or polymorph thereof. In a further aspect, the invention relates to pharmaceutical compositions comprising a pharmaceutically acceptable carrier and an effective amount of a disclosed compound, or a pharmaceutically acceptable salt, hydrate, solvate, or polymorph thereof.

Also disclosed are kits comprising at least one disclosed compound, or a pharmaceutically acceptable salt, hydrate, solvate, or polymorph thereof, and one or more of: (a) at least one agent known to increase histone demethylase activity; (b) at least one agent known to decrease histone demethylase activity; (c) at least one agent known to treat a disorder of uncontrolled cellular proliferation; (d) at least one agent known to treat a neurodegenerative disorder; (e) instructions for treating a neurodegenerative disorder; or (f) instructions for treating a disorder associated with uncontrolled cellular proliferation.

Also disclosed are methods for manufacturing a medicament comprising, combining at least one disclosed compound or at least one disclosed product with a pharmaceutically acceptable carrier or diluent. In a further aspect, the invention relates to the use of a disclosed compound in the manufacture of a medicament for the treatment of a a disorder associated with a LSD activity dysfunction. In a yet further aspect, the LSD activity dysfunction is a LSD1 activity dysfunction. In an even further aspect, the LSD activity dysfunction is a LSD2 activity dysfunction. In a still further aspect, the invention relates to the used of disclosed compound in the manufacture of a medicament for the treatment of a a disorder of uncontrolled cellular proliferation.

Also disclosed are uses of a disclosed compound or a disclosed product in the manufacture of a medicament for the treatment of a disorder associated with a LSD dysfunction in a mammal.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

FIG. 1 is a graph depicting LSD1 enzymatic activity of one of the compounds of the invention.

FIG. 2 is a graph depicting LSD1 enzymatic activity of three compounds of the invention.

FIG. 3 is a graph depicting LSD1 enzymatic activity of two compounds of the invention.

FIG. 4 is a graph depicting LSD2 enzymatic activity of four compounds of the invention.

FIG. 5 is a graph depicting LSD1 inhibition in Ewing's sarcoma cells by two compounds of the invention (SK-N-MC cells).

FIG. 6 is a graph depicting LSD1 inhibition in Ewing's sarcoma cells by two compounds of the invention (SKESI cells).

FIG. 7 is a graph depicting LSD1 inhibition in Ewing's sarcoma cells by two compounds of the invention (A673 cells).

FIG. 8 is a graph depicting the effect of administrating compounds of the invention on Ewing's sarcoma (SKNMC cells).

FIG. 9 is a graph depicting the effect of administrating compounds of the invention on Ewing's sarcoma (SKNMC cells).

### DESCRIPTION

### A. Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as ChemDraw™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the term “LSD” refers collectively to either or both LSD1 and LSD2.

As used herein, the terms “LSD1” and “lysine-specific demethylase 1” can be used interchangeably and refer to a histone demethylase encoded by the KDM1A gene. The KDM1A gene has a gene map locus of 1p36.12 as described by the Entrez Gene cytogenetic band, Ensemb1 cytogenetic band, and the HGNC cytogenetic band. The term LSD1 refers to a native protein that has 852 amino acids with a molecular weight of about 92903 Da, and is a member of the flavin monoamine oxidase family. The term LSD1 is inclusive of the protein, gene product and/or gene referred to by such alternative designations as: LSD1, KDM1; RP1-184J9.1; AOF2; BHC110; KIAA0601; LSD1; BRAF35-HDAC complex protein BHC110; FAD-binding protein BRAF35-HDAC complex, 110 kDa subunit; amine oxidase (flavin containing) domain 2; lysine-specific histone demethylase 1; lysine-specific histone demethylase 1A; flavin-containing amine oxidase domain-containing protein 2; lysine (K)-specific demethylase 1; amine oxidase (flavin containing) domain 2; and FAD-binding protein BRAF35-HDAC complex, 110 kDa subunit, as used by those skilled in the art.

As used herein, the terms “LSD2 and “lysine-specific demethylase 2 can be used interchangeably and refer to a histone demethylase encoded by the KDM1B gene. The KDM1B gene has a gene map locus of 6p22.3 as described by the Entrez Gene cytogenetic band, Ensemb1 cytogenetic band, and the HGNC cytogenetic band. The term LSD21 refers to a native protein that has 822 amino acids with a molecular weight of about 92098 Da, and is a member of the flavin monoamine oxidase family. The term LSD2 is inclusive of the protein, gene product and/or gene referred to by such alternative designations as: LSD2, AOF1; FLJ33898; FLJ34109; FLJ43328; C6orfl93; DKFZp68610412; OTTHUMP00000179125; bA204B7.3; dJ298J15.2; flavin-containing amine oxidase domain-containing protein 1; lysine-specific histone demethylase 2; lysine (K)-specific demethylase 1B; amine oxidase (flavin containing) domain 1; amine oxidase, flavin containing 1; lysine-specific histone demethylase 2; chromosome 6 open reading frame 193; and lysine-specific histone demethylase 1B, as used by those skilled in the art.

As used herein, the term “histone demethylase” refers to that group of enzymes which remove methyl groups from histone proteins. The term is inclusive of both histone lysine demethylases, i.e. enzymes which remove methyl groups from lysine residues in histones, and histone arginine demethylases, i.e. enzymes which remove methyl groups from arginine residues in histones.

As used herein, the term “histone lysine demethylase” or “lysine-specific histone demethylase” can be used interchangeably, and both refer to that group of enzymes which remove methyl groups from lysine residues of histone proteins. The histone lysine demethylases are a group of enzymes which comprise the following specific forms: LSD1, LSD2, JMJD2A, JMJD2B, JMJD2C and JMJD2D.

As used herein, the terms “optional” or “optionally” means that the subsequently described event or circumstance can or can not occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of a disorder of uncontrolled cellular proliferation associated with a histone lysine demethylase dysfunction prior to the administering step. In some aspects of the disclosed method, the subject has been diagnosed with a need for inhibition of a histone lysine demethylase prior to the administering step.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, zebra fish etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder of uncontrolled cellular proliferation” means having been subjected to a physical examination by a person of skill, for example, a physician, and found to have a condition that can be diagnosed or treated by a compound or composition that can inhibit a histone lysine demethylase. As a further example, “diagnosed with a need for inhibition of a histone demethylase” refers to having been subjected to a physical examination by a person of skill, for example, a physician, and found to have a condition characterized by a histone demethylase dysfunction. Such a diagnosis can be in reference to a disorder, such as a disorder of uncontrolled cellular proliferation, cancer and the like, as discussed herein. For example, the term “diagnosed with a need for inhibition of histone demethylase activity” refers to having been subjected to a physical examination by a person of skill, for example, a physician, and found to have a condition that can be diagnosed or treated by inhibition of histone demethylase activity. For example, “diagnosed with a need for treatment of one or more disorders of uncontrolled cellular proliferation associated with a histone demethylase dysfunction” means having been subjected to a physical examination by a person of skill, for example, a physician, and found to have one or more disorders of uncontrolled cellular proliferation associated with a histone demethylase dysfunction.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to a dysfunction of histone demethylase activity) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, intraurethral administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “EC₅₀,” is intended to refer to the concentration of a substance (e.g., a compound or a drug) that is required for 50% agonism or activation of a biological process, or component of a process, including a protein, subunit, organelle, ribonucleoprotein, etc. In one aspect, an EC₅₀ can refer to the concentration of a substance that is required for 50% agonism or activation in vivo, as further defined elsewhere herein. In a further aspect, EC₅₀ refers to the concentration of agonist or activator that provokes a response halfway between the baseline and maximum response.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. For example, an IC₅₀ can refer to the concentration of a substance that is required for 50% inhibition in vivo or the inhibition is measured in vitro, as further defined elsewhere herein. Alternatively, IC₅₀ refers to the half maximal (50%) inhibitory concentration (IC) of a substance The inhibition can be measured in a cell-line such as AN3 CA, BT-20, BT-549, HCT 116, HER218, MCF7, MDA-MB-231, MDA-MB-235, MDA-MB-435S, MDA-MB-468, PANC-1, PC-3, SK-N-MC, T-47D, and U-87 MG. In a yet further aspect, the inhibition is measured in a cell-line, e.g. HEK-293 or HeLa, transfected with a mutant or wild-type mammalian histone demethylase, e.g. LSD1 or LSD2.

The term “pharmaceutically acceptable” describes a material that is not biologically or otherwise undesirable, i.e., without causing an unacceptable level of undesirable biological effects or interacting in a deleterious manner.

The term “stable,” as used herein, refers to compounds that are not substantially altered when subjected to conditions to allow for their production, detection, and, in certain aspects, their recovery, purification, and use for one or more of the purposes disclosed herein.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

For example, a “C1-C3 alkyl” group can be selected from methyl, ethyl, n-propyl, i-propyl, and cyclopropyl, or from a subset thereof. In certain aspects, the “C1-C3 alkyl” group can be optionally further substituted. As a further example, a “C1-C4 alkyl” group can be selected from methyl, ethyl, n-propyl, i-propyl, cyclopropyl, n-butyl, i-butyl, s-butyl, t-butyl, and cyclobutyl, or from a subset thereof. In certain aspects, the “C1-C4 alkyl” group can be optionally further substituted. As a further example, a “C1-C6 alkyl” group can be selected from methyl, ethyl, n-propyl, i-propyl, cyclopropyl, n-butyl, i-butyl, s-butyl, t-butyl, cyclobutyl, n-pentyl, i-pentyl, s-pentyl, t-pentyl, neopentyl, cyclopentyl, n-hexyl, i-hexyl, 3-methylpentane, 2,3-dimethylbutane, neohexane, and cyclohexane, or from a subset thereof. In certain aspects, the “C1-C6 alkyl” group can be optionally further substituted. As a further example, a “C1-C8 alkyl” group can be selected from methyl, ethyl, n-propyl, i-propyl, cyclopropyl, n-butyl, i-butyl, s-butyl, t-butyl, cyclobutyl, n-pentyl, i-pentyl, s-pentyl, t-pentyl, neopentyl, cyclopentyl, n-hexyl, i-hexyl, 3-methylpentane, 2,3-dimethylbutane, neohexane, cyclohexane, heptane, cycloheptane, octane, and cyclooctane, or from a subset thereof. In certain aspects, the “C1-C8 alkyl” group can be optionally further substituted. As a further example, a “C1-C12 alkyl” group can be selected from methyl, ethyl, n-propyl, i-propyl, cyclopropyl, n-butyl, i-butyl, s-butyl, i-butyl, cyclobutyl, n-pentyl, i-pentyl, s-pentyl, t-pentyl, neopentyl, cyclopentyl, n-hexyl, i-hexyl, 3-methylpentane, 2,3-dimethylbutane, neohexane, cyclohexane, heptane, cycloheptane, octane, cyclooctane, nonane, cyclononane, decane, cyclodecane, undecane, cycloundecane, dodecane, and cyclododecane, or from a subset thereof. In certain aspects, the “C1-C12 alkyl” group can be optionally further substituted.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, nitrile, sulfonamide, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, nitrile, sulfonamide, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The terms “halogen,” “halide,” and “halo,” as used herein, refer to the halogens fluorine, chlorine, bromine, and iodine. It is also contemplated that, in various aspects, halogen can be selected from fluoro, chloro, bromo, and iodo. For example, halogen can be selected from fluoro, chloro, and bromo. As a further example, halogen can be selected from fluoro and chloro. As a further example, halogen can be selected from chloro and bromo. As a further example, halogen can be selected from bromo and iodo. As a further example, halogen can be selected from chloro, bromo, and iodo. In one aspect, halogen can be fluoro. In a further aspect, halogen can be chloro. In a still further aspect, halogen is bromo. In a yet further aspect, halogen is iodo.

It is also contemplated that, in certain aspects, pseudohalogens (e.g. triflate, mesylate, tosylate, brosylate, etc.) can be used in place of halogens. For example, in certain aspects, halogen can be replaced by pseudohalogen. As a further example, pseudohalogen can be selected from triflate, mesylate, tosylate, and brosylate. In one aspect, pseudohalogen is triflate. In a further aspect, pseudohalogen is mesylate. In a further aspect, pseudohalogen is tosylate. In a further aspect, pseudohalogen is brosylate.

The term “heterocycle,” as used herein refers to single and multi-cyclic aromatic or non-aromatic ring systems in which at least one of the ring members is other than carbon. Heterocycle includes azetidine, dioxane, furan, imidazole, isothiazole, isoxazole, morpholine, oxazole, oxazole, including, 1,2,3-oxadiazole, 1,2,5-oxadiazole and 1,3,4-oxadiazole, piperazine, piperidine, pyrazine, pyrazole, pyridazine, pyridine, pyrimidine, pyrrole, pyrrolidine, tetrahydrofuran, tetrahydropyran, tetrazine, including 1,2,4,5-tetrazine, tetrazole, including 1,2,3,4-tetrazole and 1,2,4,5-tetrazole, thiadiazole, including. 1,2,3-thiadiazole, 1,2,5-thiadiazole, and 1,3,4-thiadiazole, thiazole, thiophene, triazine, including 1,3,5-triazine and 1,2,4-triazine, triazole, including, 1,2,3-triazole, 1,3,4-triazole, and the like.

The term “hydroxyl” as used herein is represented by the formula —OH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and I or (+) and (−) are employed to designate the sign of rotation of plane-polarized light by the compound, with (−) or I meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture.

Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

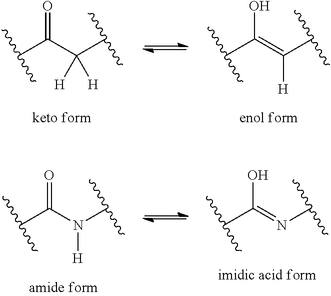 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

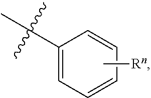

which is understood to be equivalent to a formula:

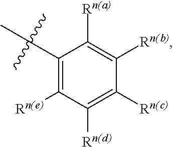

wherein n is typically an integer. That is, R″ is understood to represent five independent substituents, R^{n(a )}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Sigma-Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets. interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds useful as inhibitors of histone demethylase. In a further aspect, the compounds are useful as inhibitors of lysine-specific histone demethylase (“LSD”). Moreover, in one aspect, the compounds of the invention are useful in the treatment of disorders of uncontrolled cellular proliferations. In a further aspect, the disorder of uncontrolled cellular proliferation is a cancer or a tumor. In a still further aspect, the disorder of uncontrolled cellular proliferation is associated with a LSD dysfunction, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, the invention relates to a compound having a structure represented by formula (I):

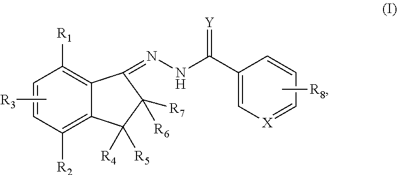

wherein

### X is CH or N;

### Y is O or S;

R₁, R₂ and R₃ are independently selected from the group consisting of hydrogen, OH, a C₁₋₆ alkyl, NH₂, a halogen, CF₃, OCF₃, O—(C₁₋₆ alkyl); and CN;
R₄, R₅, R₆ and R₇ are independently selected from the group consisting of hydrogen, a C₁₋₆ alkyl, and a halogen;

### R₈ is

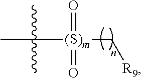

R₉ is selected from the group consisting of CH₃, NH₂, NCH₃, a C₁₋₆ alkyl, a C₁₋₆ cycloalkyl, a halogen-C₁₋₆ alkyl, a cycloalkyl, a C₁₋₆ heterocycloalkyl, aziridinyl, azetidinyl, pyrrolidinyl, piperidinyl, azepanyl, oxazolidinyl, imidazolidinyl, pyrazolidinyl, piperazinyl, oxazinanyl, morpholinyl, hexahydrophyrimidinyl, hexahydropyridazinyl and an optionally substituted moiety selected from the group consisting of:

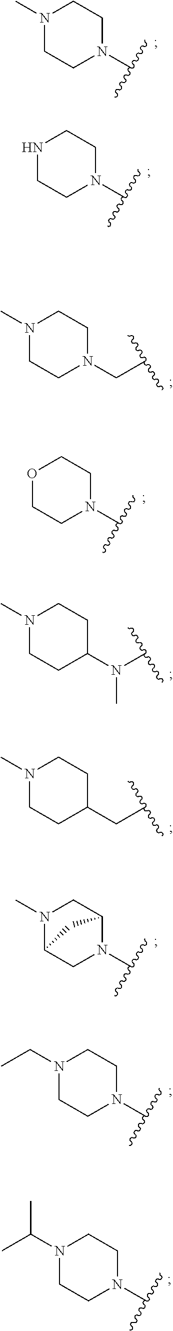

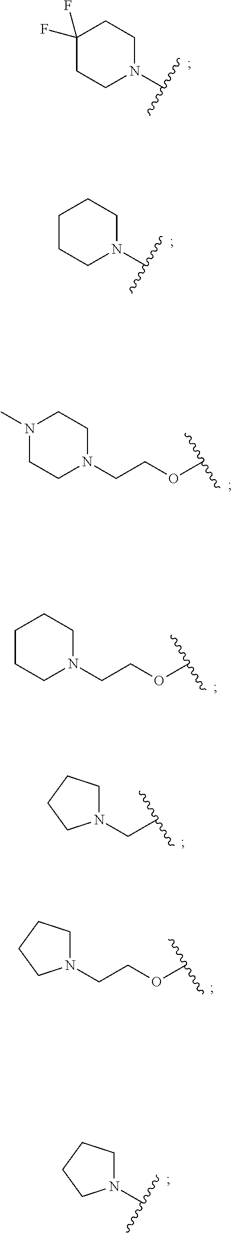

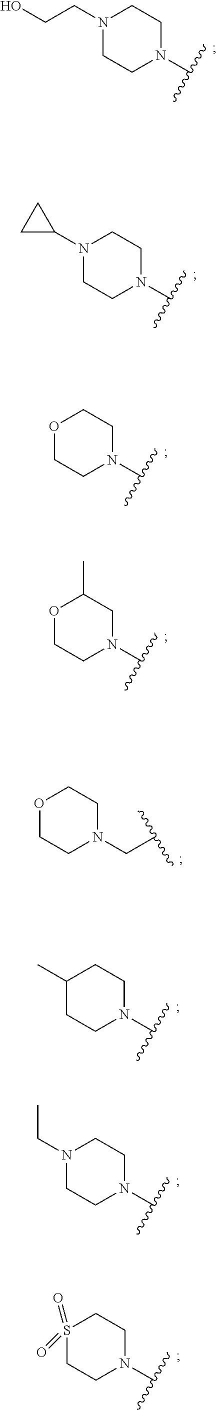

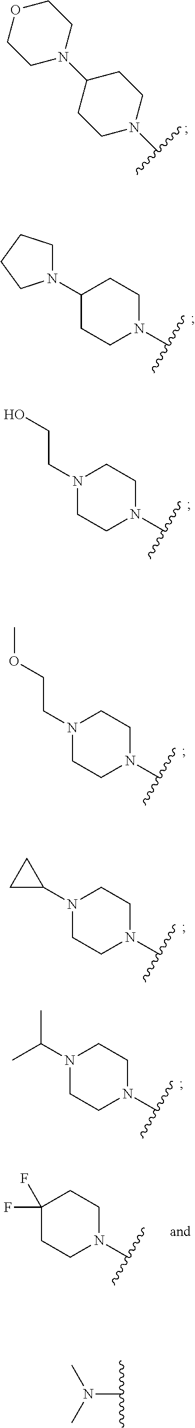

m is 0 or 1;
n is 0 or 1; with the proviso that when:
a) R₂ is a halogen; and

### b) R₃ is H; and

c) m is 1; and
d) n is 0; then
R₁ cannot be OH.
or an isomer or a pharmaceutically acceptable salt thereof.

In a preferred embodiment,

### X is CH;

### Y is O;

R₁ is selected from the group consisting of H, a halogen, an alkyl and OH;
R₂ is selected from the group consisting of H and a halogen;
R₃ is selected from the group consisting of H, OH and an alkyl;
R₄, R₅, R₆ and R₇ are H;
n is 0; and
R₉ is selected from the group consisting of:

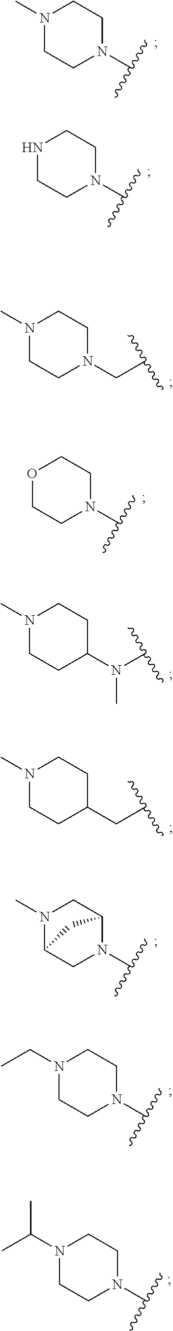

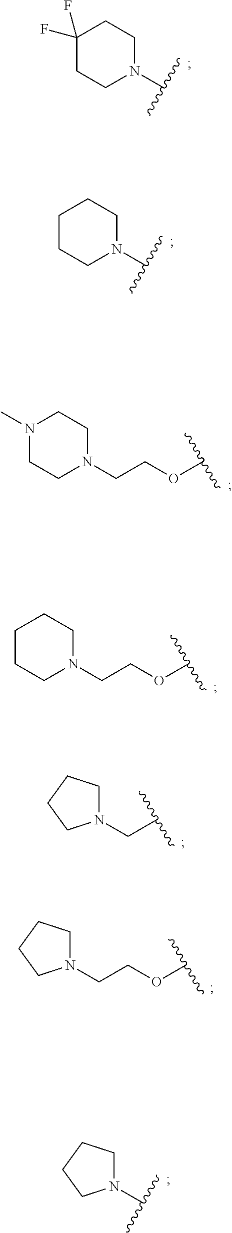

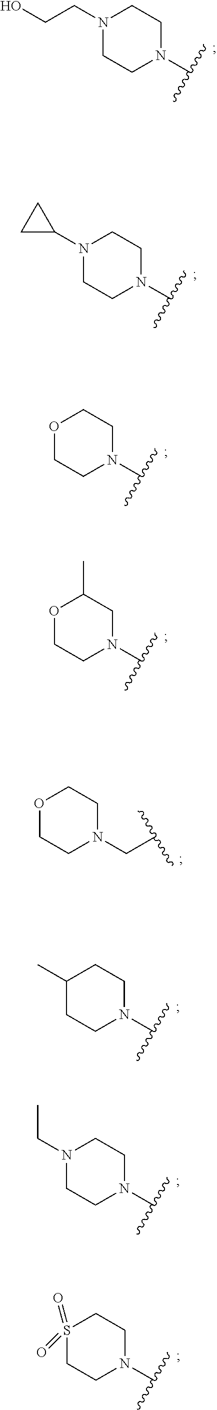

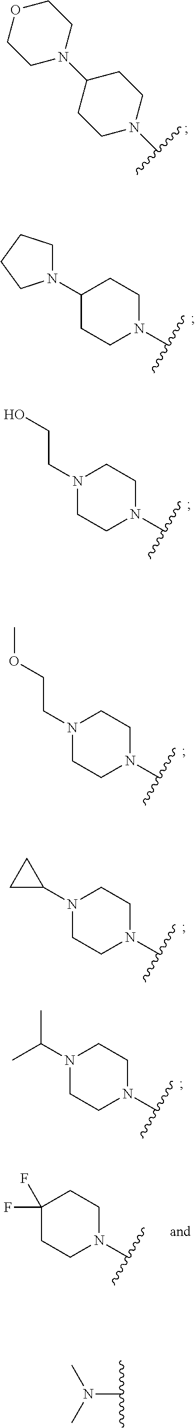

with the proviso that when:
a) R₂ is a halogen; and

### b) R₃ is H; and

c) m is 1; and
d) n is 0; then
R₁ cannot be OH.
or an isomer or a pharmaceutically acceptable salt thereof.

The following are the preferred compounds of the invention:

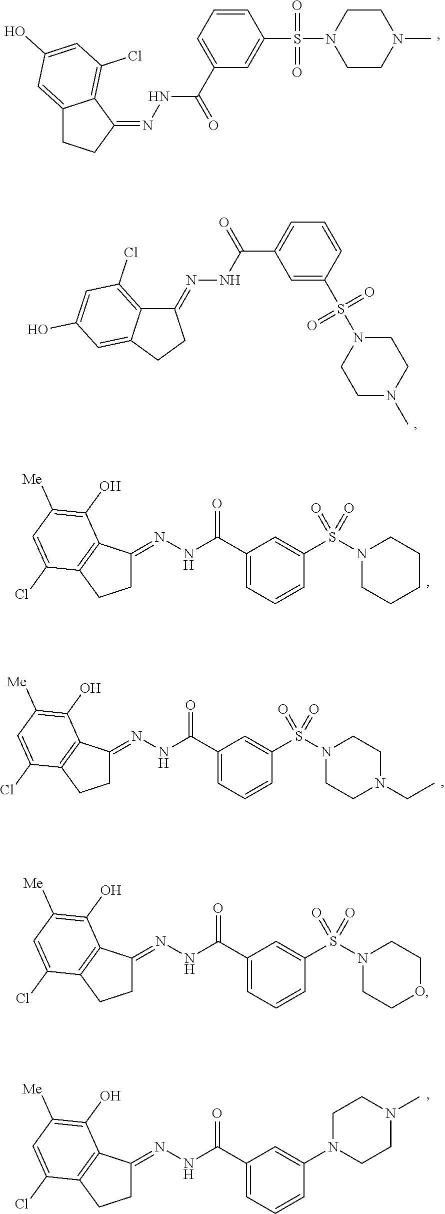 [SMILES: CN1CCN(S(=O)(=O)c2cccc(C(=O)N/N=C3/CCc4cc(O)cc(Cl)c43)c2)CC1 ; CN1CCN(S(=O)(=O)c2cccc(C(=O)N/N=C3\CCc4cc(O)cc(Cl)c43)c2)CC1 ; Cc1cc(Cl)c2c(c1O)/C(=N/NC(=O)c1cccc(S(=O)(=O)N3CCCCC3)c1)CC2 ; CCN1CCN(S(=O)(=O)c2cccc(C(=O)N/N=C3\CCc4c(Cl)cc(C)c(O)c43)c2)CC1 ; Cc1cc(Cl)c2c(c1O)/C(=N/NC(=O)c1cccc(S(=O)(=O)N3CCOCC3)c1)CC2 ; Cc1cc(Cl)c2c(c1O)/C(=N/NC(=O)c1cccc(N3CCN(C)CC3)c1)CC2]

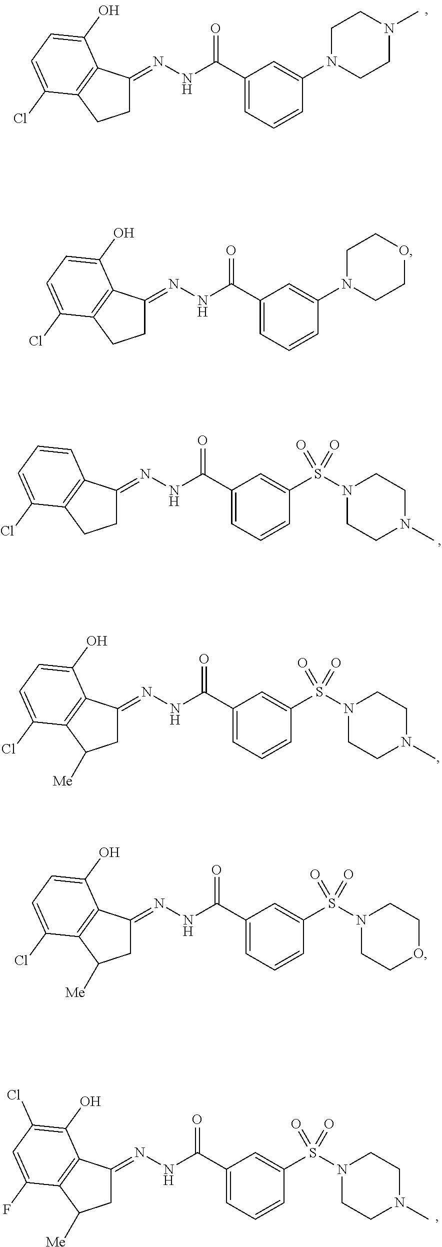 [SMILES: CN1CCN(c2cccc(C(=O)N/N=C3\CCc4c(Cl)ccc(O)c43)c2)CC1 ; O=C(N/N=C1\CCc2c(Cl)ccc(O)c21)c1cccc(N2CCOCC2)c1 ; CN1CCN(S(=O)(=O)c2cccc(C(=O)N/N=C3\CCc4c(Cl)cccc43)c2)CC1 ; CC1C/C(=N\NC(=O)c2cccc(S(=O)(=O)N3CCN(C)CC3)c2)c2c(O)ccc(Cl)c21 ; CC1C/C(=N\NC(=O)c2cccc(S(=O)(=O)N3CCOCC3)c2)c2c(O)ccc(Cl)c21 ; CC1C/C(=N\NC(=O)c2cccc(S(=O)(=O)N3CCN(C)CC3)c2)c2c(O)c(Cl)cc(F)c21]

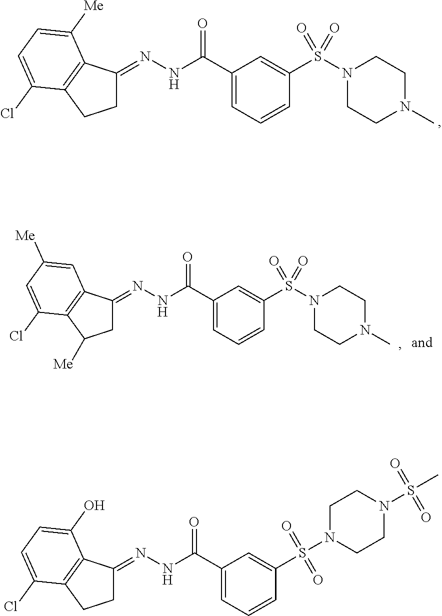 [SMILES: Cc1ccc(Cl)c2c1/C(=N/NC(=O)c1cccc(S(=O)(=O)N3CCN(C)CC3)c1)CC2 ; Cc1cc(Cl)c2c(c1)/C(=N/NC(=O)c1cccc(S(=O)(=O)N3CCN(C)CC3)c1)CC2C ; CS(=O)(=O)N1CCN(S(=O)(=O)c2cccc(C(=O)N/N=C3\CCc4c(Cl)ccc(O)c43)c2)CC1]

or an isomer or a pharmaceutically acceptable salt thereof.

The invention also provides a pharmaceutical composition comprising a therapeutically effective amount of any of a compound of the invention and a pharmaceutically acceptable carrier.

The invention also provides a method for the treatment of a disorder of uncontrolled cellular proliferation in a mammal, the method comprising the step of administering to the mammal an effective amount of any of the compounds of the invention.

The invention also provides a method for decreasing histone demethylase activity in a mammal, the method comprising the step of administering to the mammal an effective amount of any of the compounds of the invention.

The invention also provides a method for inhibiting lysine specific demethylase 1 (LSD1) activity in a mammal, the method comprising the step of administering to the mammal an effective amount of any of the compounds of the invention.

The invention also provides a method for inhibiting lysine specific demethylase 2 (LSD2) activity in a mammal, the method comprising the step of administering to the mammal an effective amount of any of the compounds of the invention.

In particular, the invention provides the following compounds that are suitable for inhibiting lysine specific demethylase 2 (LSD2) activity:

A compound having a structure represented by a formula (II):

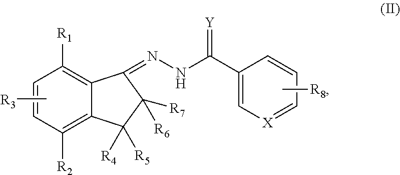

wherein

### X is CH or N;

### Y is O or S;

R₁, R₂ and R₃ are independently selected from the group consisting of hydrogen, OH, a C₁₋₆ alkyl, NH₂, a halogen, CF₃, OCF₃, O—(C₁₋₆ alkyl); and CN;
R₄, R₅, R₆ and R₇ are independently selected from the group consisting of hydrogen, a C₁₋₆ alkyl, and a halogen;

### R₈ is

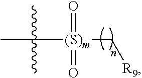

R₉ is selected from the group consisting of CH₃, NH₂, NCH₃, a C₁₋₆ alkyl, a C₁₋₆ cycloalkyl, a halogen-C₁₋₆ alkyl, a cycloalkyl, a C₁₋₆ heterocycloalkyl, aziridinyl, azetidinyl, pyrrolidinyl, piperidinyl, azepanyl, oxazolidinyl, imidazolidinyl, pyrazolidinyl, piperazinyl, oxazinanyl, morpholinyl, hexahydrophyrimidinyl, hexahydropyridazinyl and an optionally substituted moiety selected from the group consisting of:

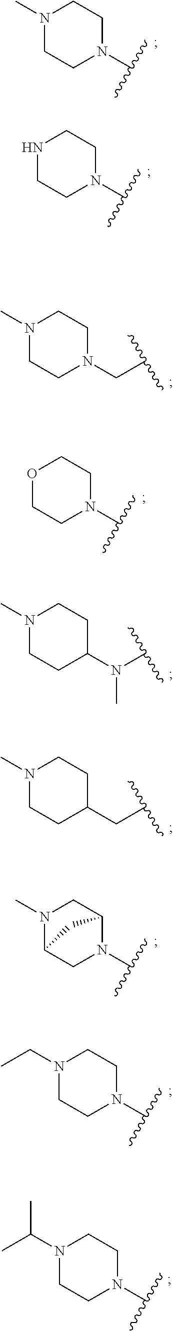

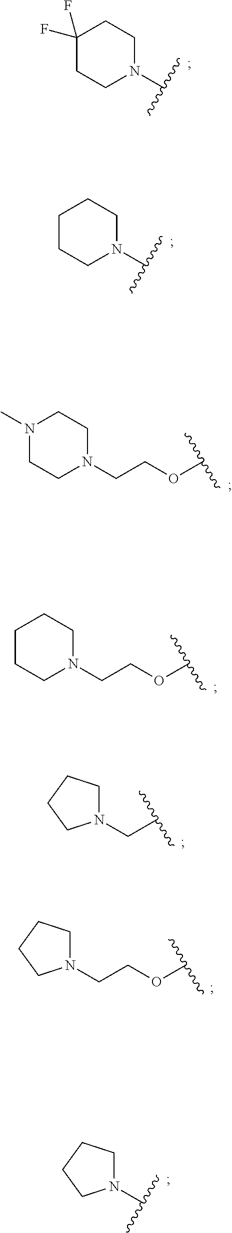

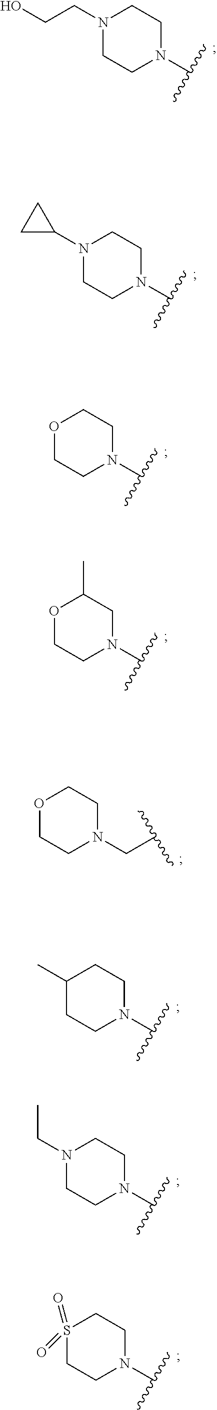

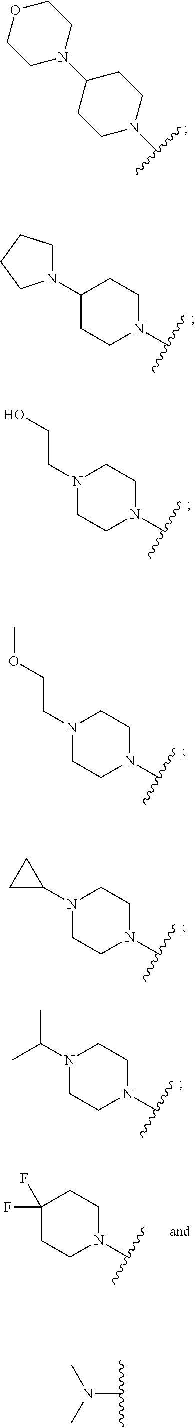

m is 0 or 1;
n is 0 or 1:

or an isomer or a pharmaceutically acceptable salt thereof.

In a preferred embodiment,

### X is CH;

### Y is O;

R₁ is selected from the group consisting of H, a halogen, an alkyl and OH;
R₂ is selected from the group consisting of H and a halogen;
R₃ is selected from the group consisting of H, OH and an alkyl;
R₄, R₅, R₆ and R₇ are H;
n is 0; and
R₉ is selected from the group consisting of:

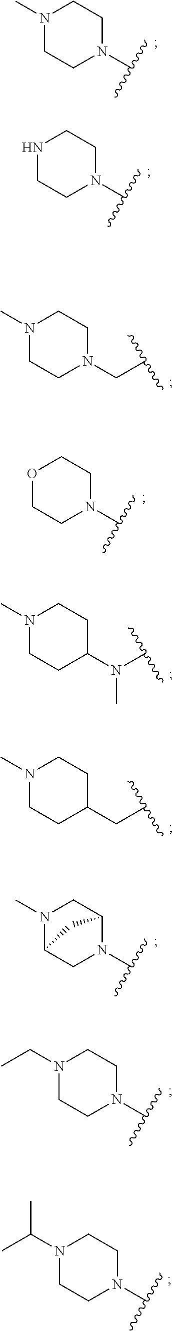

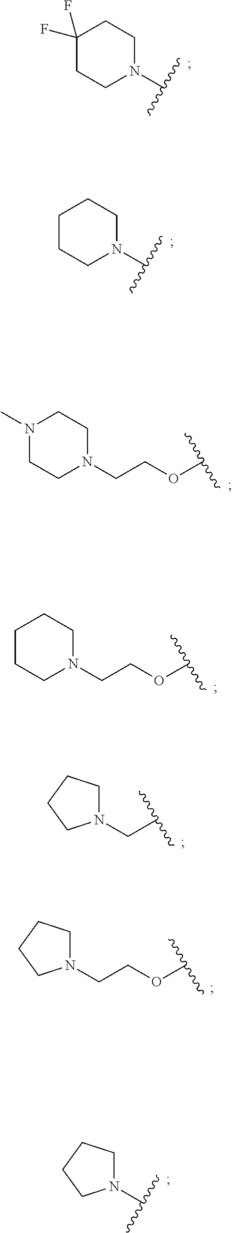

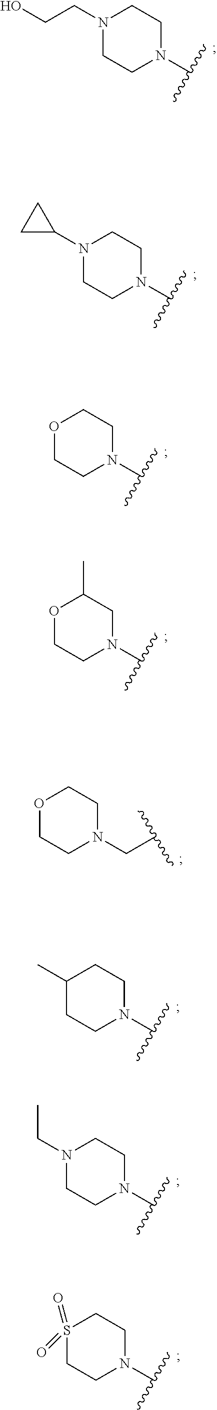

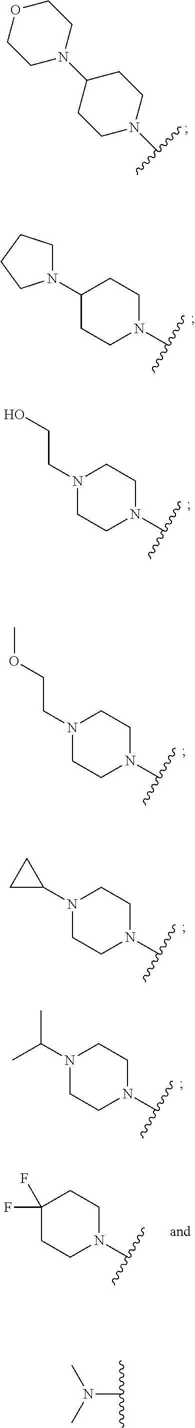

or an isomer or a pharmaceutically acceptable salt thereof.

Compounds which are particularly preferred for inhibiting LSD2 activity are the following:

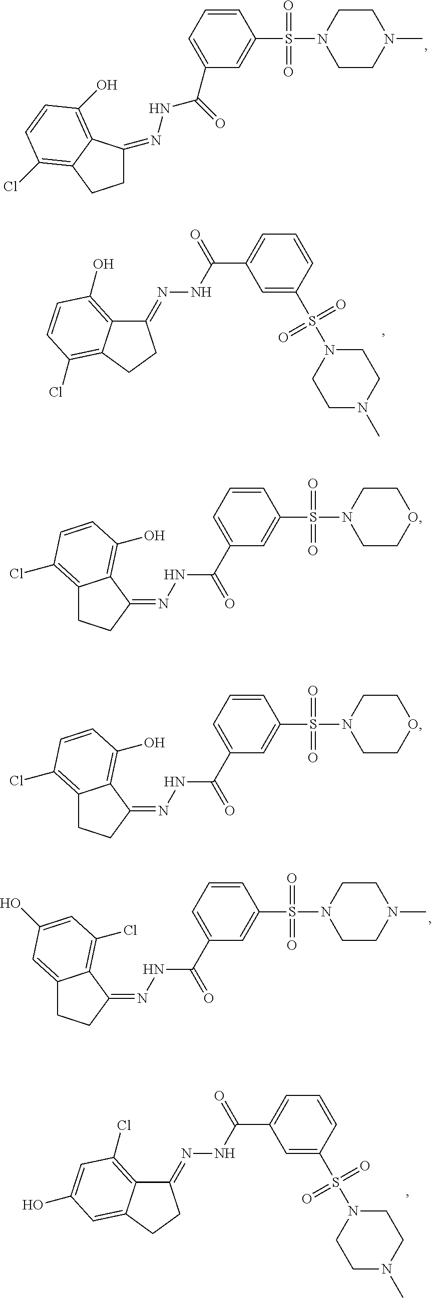 [SMILES: CN1CCN(S(=O)(=O)c2cccc(C(=O)N/N=C3/CCc4c(Cl)ccc(O)c43)c2)CC1 ; CN1CCN(S(=O)(=O)c2cccc(C(=O)N/N=C3\CCc4c(Cl)ccc(O)c43)c2)CC1 ; O=C(N/N=C1/CCc2c(Cl)ccc(O)c21)c1cccc(S(=O)(=O)N2CCOCC2)c1 ; O=C(N/N=C1/CCc2c(Cl)ccc(O)c21)c1cccc(S(=O)(=O)N2CCOCC2)c1 ; CN1CCN(S(=O)(=O)c2cccc(C(=O)N/N=C3/CCc4cc(O)cc(Cl)c43)c2)CC1 ; CN1CCN(S(=O)(=O)c2cccc(C(=O)N/N=C3\CCc4cc(O)cc(Cl)c43)c2)CC1]

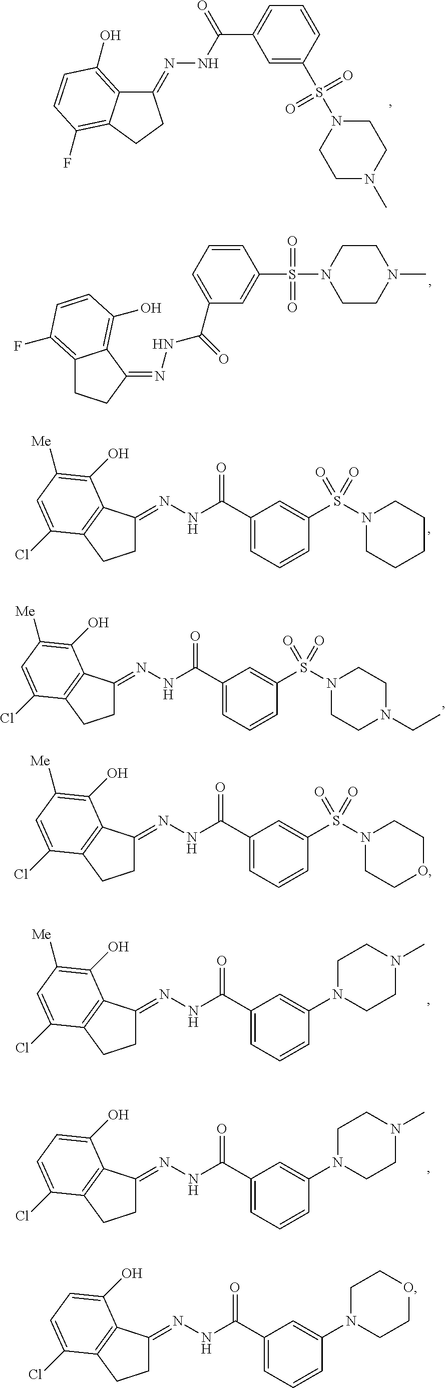

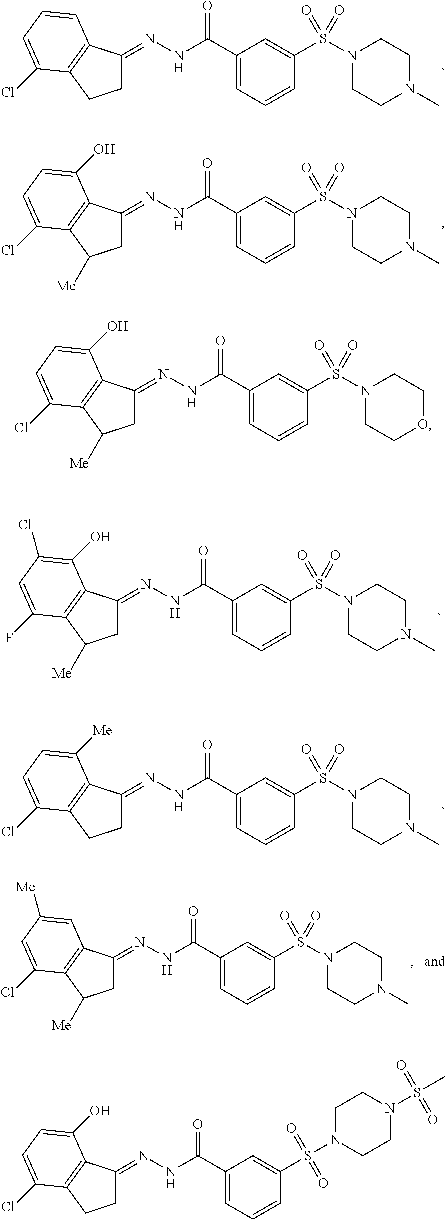

or an isomer or a pharmaceutically acceptable salt thereof.

In other words, the compounds that are suitable for inhibiting LSD2 activity include compounds which are excluded by the proviso in Formula I.

2. Inhibition of Histone Demethylase Activity

In one aspect, the disclosed compounds exhibit inhibition of LSD protein activity. In a yet further aspect, the disclosed compounds exhibit selective inhibition of LSD1 protein activity. In an even further aspect, the disclosed compounds exhibit selective inhibition of LSD2 protein activity. In a still further aspect, the disclosed compounds inhibit LSD demethylase activity. In a still further aspect, the disclosed compounds exhibit binding to the FAD domain of LSD. In an even further aspect, the disclosed compounds exhibit inhibition of LSD-mediated demethylation of histone 3 (H3) at the Lys4 position. In a still further aspect, the disclosed compounds exhibit inhibition LSD-mediated demethylation of H3K3 ml and H3K4me2. In a yet further aspect, the disclosed compounds exhibit inhibition LSD-mediated demethylation of H3K9me2 and H3K9me1.

In a still further aspect, the disclosed compounds inhibit LSD1 demethylase activity. In a still further aspect, the disclosed compounds exhibit binding to the FAD domain of LSD1. In an even further aspect, the disclosed compounds exhibit inhibition of LSD I-mediated demethylation of histone 3 (H3) at the Lys4 position. In a still further aspect, the disclosed compounds exhibit inhibition LSD1-mediated demethylation of H3K3 ml and H3K4me2. In a yet further aspect, the disclosed compounds exhibit inhibition LSD1-mediated demethylation of H3K9me2 and H3K9me1.

In a still further aspect, the disclosed compounds inhibit LSD2 demethylase activity. In a still further aspect, the disclosed compounds exhibit binding to the FAD domain of LSD2. In an even further aspect, the disclosed compounds exhibit inhibition of LSD2-mediated demethylation of histone 3 (H3) at the Lys4 position. In a still further aspect, the disclosed compounds exhibit inhibition LSD2-mediated demethylation of H3K3 m1 and H3K4me2.

In a further aspect, the disclosed compounds exhibit disruption of of LSD interaction with a complexes comprising one or more of HDAC1/2, CoREST, CtBP1, BRAF35 and BHC80 proteins. In a still further aspect, the disclosed compounds disrupt binding of LSD1 to one or more proteins selected from HDAC1/2, CoREST, CtBP1, BRAF35 and BHC80 proteins. In a still further aspect, the disclosed compounds disrupt binding of LSD2 to one or more proteins selected from G9a, NSD3, HDAC1/2, CoREST, CtBP1, BRAF35 and BHC80 proteins.

Inhibition of LSD activity can be determined by a variety of both in vitro and in vivo methods known to one skilled in the art. For example, enzymatic activity can be determined in in vitro enzyme assay systems. In various aspects, the enzymatic activity of either LSD1 or LSD2 can be determined in a spectrophometric assay. Briefly, the assay is based on the multistep enzymatic reaction in which LSD1 or LSD2 first produces H₂O₂ during the demethylation of lysine 4 on a peptide corresponding to the first 21 amino acids of the N-terminal tail of histone H3. In the presence of horseradish peroxidase, the H₂O₂ produced reacts with ADHP to produce the highly fluorescent compound resorufin that can be analyzed with an excitation wavelength of 530-540 nm and an emission wavelength of 585-595 nm. The assay requires a source of LSD or LSD2 enzyme, either purified from natural sources (e.g. a tissue or cultured cells), isolated as a recombinantly expressed protein, or as a unpurified protein in whole cell extracts. In one aspect, the disclosed compounds exhibit inhibition of LSD protein activity with an IC₅₀ in an EMSA assay of less than about about 300 μM, less than about about 100 μM, less than about 50 μM, less than about 10 μM, less than about 1 μM, less than about 500 nM, or of less than about 100 nM. In a further aspect, the disclosed compounds exhibit inhibition of LSD1 protein activity with an IC₅₀ in an EMSA assay of less than about about 300 μM, less than about about 100 μM, less than about 50 μM, less than about 10 μM, less than about 1 μM, less than about 500 nM, or of less than about 100 nM. In a still further aspect, the disclosed compounds exhibit inhibition of LSD2 protein activity with an IC₅₀ in an EMSA assay of less than about about 300 μM, less than about about 100 μM, less than about 50 μM, less than about 10 μM, less than about 1 μM, less than about 500 nM, or of less than about 100 nM.

In one aspect, the disclosed compounds are selective for LSD. In a further aspect, selective inhibition of LSD activity is determined using an enzyme assay. In various further aspects, the compound inhibits LSD activity in an enzyme assay with an IC₅₀ less than the IC₅₀ for MAO A and/or MAO B. That is, a disclosed compound can have selectivity for the LSD protein vis-à-vis MAO A and/or MAO B. For example, in one aspect, a disclosed compound can inhibit LSD with an IC₅₀ of about 5-fold less than that for MAO A, of about 10-fold less than that for MAO A, of about 20-fold less than that for MAO A, of about 30-fold less than that for MAO A, of about 50-fold less than that for MAO A, of about 100-fold less than that for MAO A, of about 250-fold less than that for MAO A, of about 500-fold less than that for MAO A, of about 1000-fold less than that for MAO A, and more than about 1000-fold less than that for MAO A. In a further aspect, a disclosed compound can inhibit LSD with an IC₅₀ of about 5-fold less than that for MAO B, of about 10-fold less than that for MAO B, of about 20-fold less than that for MAO B, of about 30-fold less than that for MAO B, of about 50-fold less than that for MAO B, of about 100-fold less than that for MAO B, of about 250-fold less than that for MAO B, of about 500-fold less than that for MAO B, of about 1000-fold less than that for MAO B, and more than about 1000-fold less than that for MAO B.

In one aspect, the disclosed compounds are selective for LSD1. In a further aspect, selective inhibition of LSD1 activity is determined using an enzyme assay. In various further aspects, the compound inhibits LSD1 activity in an enzyme assay with an IC₅₀ less than the IC₅₀ for one or more of LSD2, MAO A, and MAO B. That is, a disclosed compound can have selectivity for the LSD1 protein vis-à-vis one or more of of LSD2, MAO A, and MAO B. For example, in one aspect, a disclosed compound can inhibit LSD1 with an IC₅₀ of about 5-fold less than that for LSD2, of about 10-fold less than that for LSD2, of about 20-fold less than that for LSD2, of about 30-fold less than that for LSD2, or of about 50-fold less than that for LSD2. In a further aspect, a disclosed compound can inhibit LSD1 with an IC₅₀ of about 5-fold less than that for MAO A, of about 10-fold less than that for MAO A, of about 20-fold less than that for MAO A, of about 30-fold less than that for MAO A, of about 50-fold less than that for MAO A, of about 100-fold less than that for MAO A, of about 250-fold less than that for MAO A, of about 500-fold less than that for MAO A, of about 1000-fold less than that for MAO A, and more than about 1000-fold less than that for MAO A. In a further aspect, a disclosed compound can inhibit LSD1 with an IC₅₀ of about 5-fold less than that for MAO B, of about 10-fold less than that for MAO B, of about 20-fold less than that for MAO B, of about 30-fold less than that for MAO B, of about 50-fold less than that for MAO B, of about 100-fold less than that for MAO B, of about 250-fold less than that for MAO B, of about 500-fold less than that for MAO B, of about 1000-fold less than that for MAO B, and more than about 1000-fold less than that for MAO B.

In one aspect, the disclosed compounds are selective for LSD2. In a further aspect, selective inhibition of LSD2 activity is determined using an enzyme assay. In various further aspects, the compound inhibits LSD2 activity in an enzyme assay with an IC₅₀ less than the IC₅₀ for one or more of LSD1, MAO A, and MAO B. That is, a disclosed compound can have selectivity for the LSD2 protein vis-à-vis one or more of of LSD1, MAO A, and MAO B. For example, in one aspect, a disclosed compound can inhibit LSD2 with an IC₅₀ of about 5-fold less than that for LSD1, of about 10-fold less than that for LSD1, of about 20-fold less than that for LSD1, of about 30-fold less than that for LSD1, or of about 50-fold less than that for LSD1. In a further aspect, a disclosed compound can inhibit LSD2 with an IC₅₀ of about 5-fold less than that for MAO A, of about 10-fold less than that for MAO A, of about 20-fold less than that for MAO A, of about 30-fold less than that for MAO A, of about 50-fold less than that for MAO A, of about 100-fold less than that for MAO A, of about 250-fold less than that for MAO A, of about 500-fold less than that for MAO A, of about 1000-fold less than that for MAO A, and more than about 1000-fold less than that for MAO A. In a further aspect, a disclosed compound can inhibit LSD2 with an IC₅₀ of about 5-fold less than that for MAO B, of about 10-fold less than that for MAO B, of about 20-fold less than that for MAO B, of about 30-fold less than that for MAO B, of about 50-fold less than that for MAO B, of about 100-fold less than that for MAO B, of about 250-fold less than that for MAO B, of about 500-fold less than that for MAO B, of about 1000-fold less than that for MAO B, and more than about 1000-fold less than that for MAO B.

In various aspects, the disclosed compounds exhibit binding to a LSD protein. In a further aspect, the disclosed compounds exhibit binding to the FAD domain of a LSD protein. In a still further aspect, the disclosed compounds exhibit binding to LSD1 protein. In an even further aspect, the disclosed compounds exhibit binding to LSD2 protein. The binding affinity of a disclosed compound for a LSD protein, e.g. LSD1 protein, can be determined by various methods known to one skilled in the art. In one aspect, the disclosed compounds exhibit binding to LSD protein with a K_{D} of less than about about 50 μM, less than about 10 μM, less than about 1 μM, less than about 500 nM, or of less than about 100 nM. In a further aspect, the K_{D} is determined using an SPR method. In a still further aspect, the binding is determined using LSD1 protein. In a yet further aspect, the binding is determined using LSD2 protein.

In various further aspects, the binding to LSD is selective. In a further aspect, the disclosed compounds exhibit a K_{D} for LSD binding less than the K_{D} of MAO A and/or MAO B. That is, a disclosed compound can have selectivity for the LSD protein vis-à-vis MAO A and/or MAO B proteins. For example, in one aspect, a disclosed compound can bind LSD with a K_{D} of about 5-fold less than that for MAO A, of about 10-fold less than that for MAO A, of about 20-fold less than that for MAO A, of about 30-fold less than that for MAO A, of about 50-fold less than that for MAO A, of about 100-fold less than that for MAO A, of about 250-fold less than that for MAO A, of about 500-fold less than that for MAO A, of about 1000-fold less than that for MAO A, and of more than about 1000-fold less than that for MAO A. In a further aspect, a disclosed compound can bind LSD with a K_{D} of about 5-fold less than that for MAO B, of about 10-fold less than that for MAO B, of about 20-fold less than that for MAO B, of about 30-fold less than that for MAO B, of about 50-fold less than that for MAO B, of about 100-fold less than that for MAO B, of about 250-fold less than that for MAO B, of about 500-fold less than that for MAO B, of about 1000-fold less than that for MAO B, and of more than about 1000-fold less than that for MAO B.

In various further aspects, the binding to LSD1 is selective. In a further aspect, the disclosed compounds exhibit a K_{D} for LSD1 binding less than the K_{D} for one or more of LSD2, MAO A, and MAO B. That is, a disclosed compound can have selectivity for the LSD1 protein vis-à-vis one or more of of LSD2, MAO A, and MAO B proteins. For example, in one aspect, a disclosed compound can bind LSD1 with a K_{D} of about 5-fold less than that for LSD2, of about 10-fold less than that for LSD2, of about 20-fold less than that for LSD2, of about 30-fold less than that for LSD2, or of about 50-fold less than that for LSD2. In a further aspect, a disclosed compound can bind LSD1 with a K_{D} of about 5-fold less than that for MAO A, of about 10-fold less than that for MAO A, of about 20-fold less than that for MAO A, of about 30-fold less than that for MAO A, of about 50-fold less than that for MAO A, of about 100-fold less than that for MAO A, of about 250-fold less than that for MAO A, of about 500-fold less than that for MAO A, of about 1000-fold less than that for MAO A, and of more than about 1000-fold less than that for MAO A. In a further aspect, a disclosed compound can bind LSD1 with a K_{D} of about 5-fold less than that for MAO B, of about 10-fold less than that for MAO B, of about 20-fold less than that for MAO B, of about 30-fold less than that for MAO B, of about 50-fold less than that for MAO B, of about 100-fold less than that for MAO B, of about 250-fold less than that for MAO B, of about 500-fold less than that for MAO B, of about 1000-fold less than that for MAO B, and of more than about 1000-fold less than that for MAO B.

In various further aspects, the binding to LSD2 is selective. In a further aspect, the disclosed compounds exhibit a K_{D} for LSD2 binding less than the K_{D} for one or more of LSD1, MAO A, and MAO B. That is, a disclosed compound can have selectivity for the LSD2 protein vis-à-vis one or more of of LSD1, MAO A, and MAO B proteins. For example, in one aspect, a disclosed compound can bind LSD2 with a K_{D} of about 5-fold less than that for LSD1, of about 10-fold less than that for LSD1, of about 20-fold less than that for LSD1, of about 30-fold less than that for LSD1, or of about 50-fold less than that for LSD. In a further aspect, a disclosed compound can bind LSD2 with a K_{D} of about 5-fold less than that for MAO A, of about 10-fold less than that for MAO A, of about 20-fold less than that for MAO A, of about 30-fold less than that for MAO A, of about 50-fold less than that for MAO A, of about 100-fold less than that for MAO A, of about 250-fold less than that for MAO A, of about 500-fold less than that for MAO A, of about 1000-fold less than that for MAO A, and of more than about 1000-fold less than that for MAO A. In a further aspect, a disclosed compound can bind LSD2 with a K_{D} of about 5-fold less than that for MAO B, of about 10-fold less than that for MAO B, of about 20-fold less than that for MAO B, of about 30-fold less than that for MAO B, of about 50-fold less than that for MAO B, of about 100-fold less than that for MAO B, of about 250-fold less than that for MAO B, of about 500-fold less than that for MAO B, of about 1000-fold less than that for MAO B, and of more than about 1000-fold less than that for MAO B.

Alternatively, the inhibition of STAT protein activity can be determined in a cell-based assay. There are a variety of cell-based assays that are suitable for determination of inhibition of LSD protein activity known to one skilled in the art. For example, cell growth inhibition or cell arrest can be determined using a cell, either a permanent cell-line or a primary cell culture that has a LSD protein with dysfunction activity. In a further aspect, the LSD protein is LSD1. In a still further aspect, the LSD protein is LSD2. In a yet further aspect, the LSD protein dysfunction is one wherein the LSD protein is has acquired a gain of function mutation. Alternatively, the LSD protein dysfunction has a phenotype of persistent or constitutive activity. For example, the LSD protein can have a persistent or constitutive activity due to a dysfunction in an upstream regulatory protein. In a further aspect, the LSD protein is overexpressed due to a dysfunction in regulation of transcription and/or translation of the LSD gene. In a further aspect, the cell harbors an active oncogene is associated with LSD dysfunction.

In one aspect, the disclosed compounds and products of disclosed methods of making inhibit cell growth. In a still further aspect, the disclosed compounds and products of disclosed methods inhibit cell growth in an in vitro assay system. In an even further aspect, the in vitro assay system makes use of a cell-line derived from a from cancer or tumor selected from breast cancer, ovarian cancer, testicular cancer, lung cancer, liver cancer, prostate cancer, pancreatic cancer and a sarcoma. In a yet further aspect, the cell-line is derived from a human source. In a yet further aspect, the disclosed compounds inhibit cell growth in a cell with a persistently active LSD protein. In an even further aspect, the cell-line has an activated LSD protein. In a still further aspect, the cell-line is selected from AN3 CA, BT-20, BT-549, HCT 116, HER218, MCF7, MDA-MB-231, MDA-MB-235, MDA-MB-435S, MDA-MB-468, PANC-1, PC-3, SK-N-MC, T-47D, and U-87 MG. In one aspect, the disclosed compounds exhibit inhibition of cell growth activity in an in vitro cell-based assay with an IC₅₀ of less than about about 500 μM, of less than about about 250 μM, less than about about 100 μM, less than about 50 μM, less than about 10 μM, less than about 1 μM, less than about 500 nM, of less than about 100 nM, of less than about 10 nM, and of less than about 1 nM.

In one aspect, the disclosed compounds and products of disclosed methods of making inhibit cell migration. In a still further aspect, the disclosed compounds and products of disclosed methods inhibit cell migration in an in vitro assay system. In an even further aspect, the in vitro assay system makes use of a cell-line derived from a from cancer or tumor selected from breast cancer, ovarian cancer, testicular cancer, lung cancer, liver cancer, prostate cancer, pancreatic cancer and a sarcoma. In a yet further aspect, the cell-line is derived from a human source. In a yet further aspect, the disclosed compounds inhibit cell growth in a cell with a persistently active LSD protein. In an even further aspect, the cell-line has an activated LSD protein. In a still further aspect, the cell-line is selected from AN3 CA, BT-20, BT-549, HCT 116, HER218, MCF7, MDA-MB-231, MDA-MB-235, MDA-MB-435S, MDA-MB-468, PANC-1, PC-3, SK-N-MC, T-47D, and U-87 MG. In one aspect, the disclosed compounds exhibit inhibition of cell migration in an in vitro cell-based assay with an IC₅₀ of less than about about 300 μM, less than about about 100 μM, less than about 50 μM, less than about 10 μM, less than about 1 μM, less than about 500 nM, or of less than about 100 nM.

### C. Methods of Making the Compounds

In one aspect, the invention relates to methods of making compounds useful as inhibitors of LSD. In a further aspect, the products of disclosed methods of making are modulators of LSD activity. In a yet further aspect, the products of disclosed methods of making bind to a STAT protein and negatively modulate LSD activity. The compounds can, in one aspect, exhibit subtype selectivity. In a still further aspect, the products of disclosed methods of making exhibit selectivity for the LSD1 member of the LSD protein family. In an even further aspect, the products of the disclosed methods of making exhibit selectivity for the LSD2 member of the LSD protein family.

In one aspect, the invention relates to methods of making compounds useful as inhibitors of histone demethylase, which can be useful in the treatment of disorders of uncontrolled cellular proliferation. In a further aspect, the histone demethylase is LSD1. In a yet further aspect, the histone demethylase is LSD2.

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, in addition to other standard manipulations known in the literature or to one skilled in the art. The following examples are provided so that the invention might be more fully understood, are illustrative only, and should not be construed as limiting.

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

1. Building Blocks

In one aspect, the compounds of the present invention can be prepared utilizing the following compounds as “building blocks”:

### Indanones and Ketone

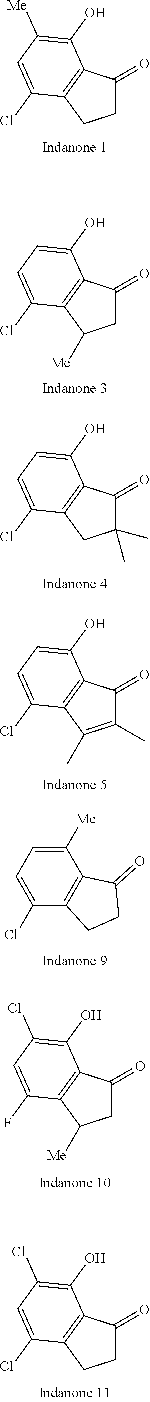

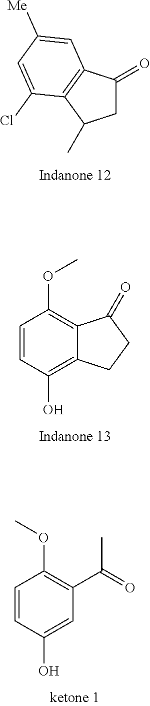

### Hydrazide Intermediates

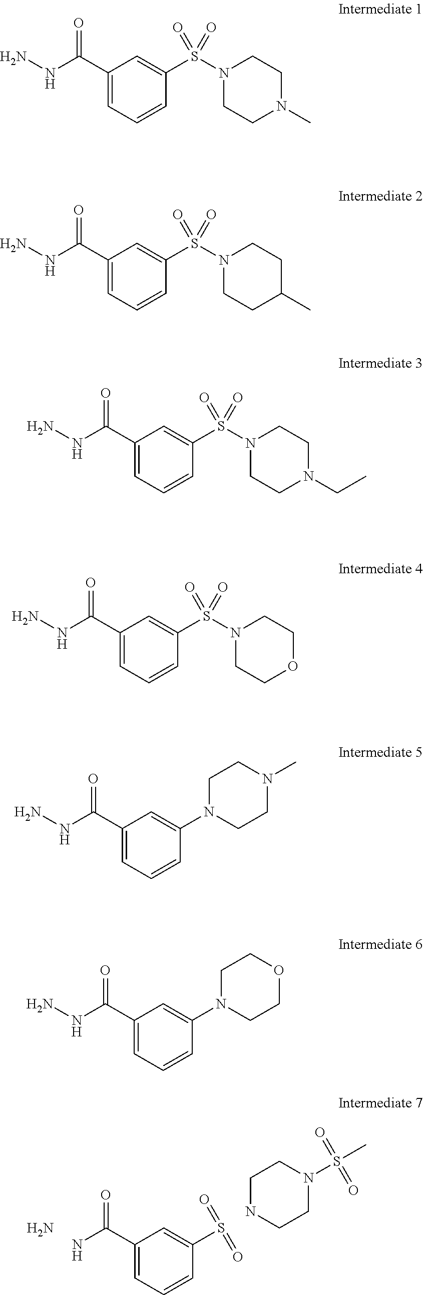

The following generic schemes can be used to make compounds of the invention:

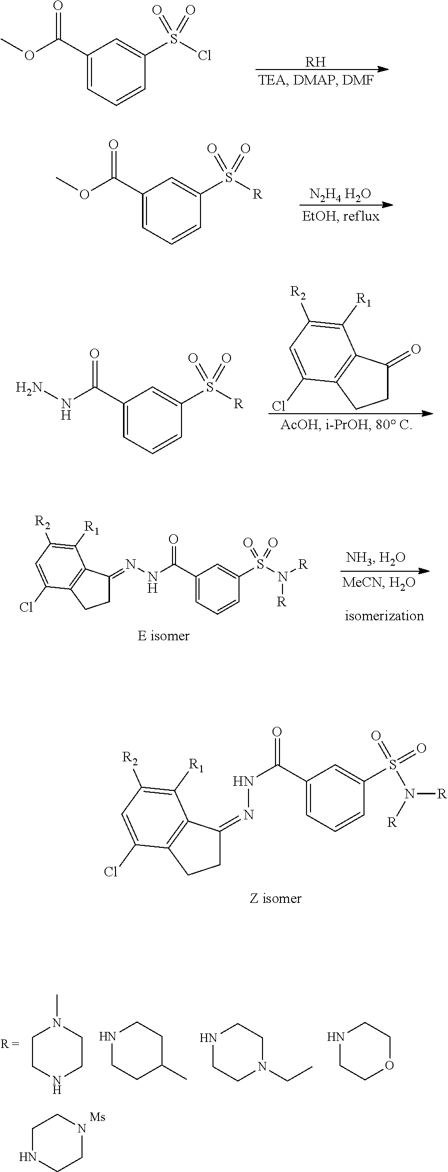

It is contemplated that each disclosed methods can further comprise additional steps, manipulations, and/or components. It is also contemplated that any one or more step, manipulation, and/or component can be optionally omitted from the invention. It is understood that a disclosed methods can be used to provide the disclosed compounds. It is also understood that the products of the disclosed methods can be employed in the disclosed methods of using.

### D. Pharmaceutical Compositions

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In a further aspect, the invention relates to pharmaceutical compositions comprising a pharmaceutically acceptable carrier and an effective amount of the product of a disclosed synthetic method. In a further aspect, the effective amount is a therapeutically effective amount. In a further aspect, the effective amount is a prophylactically effective amount. In a further aspect, the compound is a disclosed compound.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in moulds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment conditions which require inhibition or negative modulation of LSD protein activity an appropriate dosage level will generally be about 0.01 to 500 mg per kg patient body weight per day and can be administered in single or multiple doses. Preferably, the dosage level will be about 0.1 to about 250 mg/kg per day; more preferably 0.5 to 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the from of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

The present invention is further directed to a method for the manufacture of a medicament for inhibiting or negatively modulating LSD protein activity (e.g., treatment of a disorder of uncontrolled cellular proliferation, or one or more neurodegenerative disorders associated with LSD dysfunction) in mammals (e.g., humans) comprising combining one or more disclosed compounds, products, or compositions with a pharmaceutically acceptable carrier or diluent. Thus, in one aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

### E. Methods of Using the Compounds and Compositions

The disclosed compounds can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

The pharmaceutical compositions and methods of the present invention can further comprise other therapeutically active compounds as noted herein which are usually applied in the treatment of the above mentioned pathological conditions.

1. Treatment Methods

The compounds disclosed herein are useful for treating, preventing, ameliorating, controlling or reducing the risk of a variety of disorders wherein the patient or subject would benefit from inhibition or negative modulation of a LSD protein. In one aspect, a treatment can include selective inhibition of LSD to an extent effective to affect histone demethylation activity. Thus, a disorder can be associated with histone demethylation activity, for example dysfunctional epigenetic regulation of genes in a cancer cell. In one aspect, provided is a method of treating or preventing a disorder in a subject comprising the step of administering to the subject at least one disclosed compound; at least one disclosed pharmaceutical composition; and/or at least one disclosed product in a dosage and amount effective to treat the disorder in the subject.

Also provided is a method for the treatment of one or more disorders, for which LSD inhibition is predicted to be beneficial, in a subject comprising the step of administering to the subject at least one disclosed compound; at least one disclosed pharmaceutical composition; and/or at least one disclosed product in a dosage and amount effective to treat the disorder in the subject.

In one aspect, provided is a method for treating a disorder of uncontrolled cellular proliferation, comprising: administering to a subject at least one disclosed compound; at least one disclosed pharmaceutical composition; and/or at least one disclosed product in a dosage and amount effective to treat the disorder in the subject. In a further aspect, provided is a method for treating or preventing a neurodegenerative disorder, comprising: administering to a subject at least one disclosed compound; at least one disclosed pharmaceutical composition; and/or at least one disclosed product in a dosage and amount effective to treat the disorder in the subject. Also provided is a method for the treatment of a disorder in a mammal comprising the step of administering to the mammal at least one disclosed compound, composition, or medicament.

The invention is directed at the use of described chemical compositions to treat diseases or disorders in patients (preferably human) wherein wherein LSD inhibition would be predicted to have a therapeutic effect, such as disorders of uncontrolled cellular proliferation (e.g. cancers) and neurodegenerative disorders such as Alzheimer's disease, Huntington's disease, and Parkinson's disease, by administering one or more disclosed compounds or products.

The compounds disclosed herein are useful for treating, preventing, ameliorating, controlling or reducing the risk of a variety of disorders of uncontrolled cellular proliferation. In one aspect, the disorder of uncontrolled cellular proliferation is associated with a histone demethylase dysfunction. In a further aspect, the histone demethylase dysfunction is disregulation of the LSD. In a still further aspect, the histone demethylase dysfunction is disregulation of the LSD1. In an even further aspect, the histone demethylase dysfunction is disregulation of the LSD2.

Also provided is a method of use of a disclosed compound, composition, or medicament. In one aspect, the method of use is directed to the treatment of a disorder. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

Examples of disorders associated with a histone demethylase dysfunction include a disorder of uncontrolled cellular proliferation. In a yet further aspect, the disorder of uncontrolled cellular proliferation is cancer. In a yet further aspect, the cancer is a leukemia. In an even further aspect, the cancer is a sarcoma. In a still further aspect, the cancer is a solid tumor. In a yet further aspect, the cancer is a lymphoma.

It is understood that cancer refer to or describe the physiological condition in mammals that is typically characterized by unregulated cell growth. The cancer may be multi-drug resistant (MDR) or drug-sensitive. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer.

In various aspects, further examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas

In a further aspect, the cancer is a hematological cancer. In a still further aspect, the hematological cancer is selected from acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, and extramedullary myeloma. In a still further aspect, the cancer is selected from chronic lymphocytic leukemia, small lymphocytic lymphoma, B-cell non-Hodgkin lymphoma, and large B-cell lymphoma.

In a further aspect, the cancer is a cancer of the brain. In a still further aspect, the cancer of the brain is selected from a glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, and metastatic brain tumor. In a yet further aspect, the glioma is selected from ependymoma, astrocytoma, oligodendroglioma, and oligoastrocytoma. In an even further aspect, the glioma is selected from juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, and ganglioglioma.

In one aspect, the cancer can be a cancer selected from cancers of the blood, brain, genitourinary tract, gastrointestinal tract, colon, rectum, breast, kidney, lymphatic system, stomach, lung, pancreas, and skin. In a further aspect, the cancer is selected from prostate cancer, glioblastoma multiforme, endometrial cancer, breast cancer, and colon cancer. In a further aspect, the cancer is selected from a cancer of the breast, ovary, prostate, head, neck, and kidney. In a still further aspect, the cancer is selected from cancers of the blood, brain, genitourinary tract, gastrointestinal tract, colon, rectum, breast, livery, kidney, lymphatic system, stomach, lung, pancreas, and skin. In a yet further aspect, the cancer is selected from a cancer of the lung and liver. In an even further aspect, the cancer is selected from a cancer of the breast, ovary, testes and prostate In a still further aspect, the cancer is a cancer of the breast. In a yet further aspect, the cancer is a cancer of the ovary. In an even further aspect, the cancer is a cancer of the prostate. In a still further aspect, the cancer is a cancer of the testes.

In various aspects, disorders associated with a histone demethylase dysfunction include neurodegenerative disorders. In a further aspect, the neurodegenerative disease is selected from Alzheimer's disease, Parkinson's disease, and Huntington's disease.

The compounds are further useful in a method for the prevention, treatment, control, amelioration, or reducation of risk of the diseases, disorders and conditions noted herein. The compounds are further useful in a method for the prevention, treatment, control, amelioration, or reduction of risk of the aforementioned diseases, disorders and conditions in combination with other agents.

The present invention is further directed to administration of a LSD inhibitor for improving treatment outcomes in the context of disorders of uncontrolled cellular proliferation, including cancer. That is, in one aspect, the invention relates to a cotherapeutic method comprising the step of administering to a mammal an effective amount and dosage of at least one compound of the invention in connection with cancer therapy.

In a further aspect, administration improves treatment outcomes in the context of cancer therapy. Administration in connection with cancer therapy can be continuous or intermittent. Administration need not be simultaneous with therapy and can be before, during, and/or after therapy. For example, cancer therapy can be provided within 1, 2, 3, 4, 5, 6, 7 days before or after administration of the compound. As a further example, cancer therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

In one aspect, the disclosed compounds can be used in combination with one or more other drugs in the treatment, prevention, control, amelioration, or reduction of risk of diseases or conditions for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly.

Accordingly, the pharmaceutical compositions include those that contain one or more other active ingredients, in addition to a compound of the present invention.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the subject compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In one aspect, the compound can be employed in combination with anti-cancer therapeutic agents or other known therapeutic agents.

In the treatment of conditions which require inhibition or negative modulation of LSD, an appropriate dosage level will generally be about 0.01 to 1000 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Thus, in one aspect, the invention relates to methods for inhibiting or negatively modulating LSD in at least one cell, comprising the step of contacting the at least one cell with at least one compound of the invention, in an amount effective to modulate or activate LSD activity response, e.g. LSD1 or LSD2, in the at least one cell. In a further aspect, the cell is mammalian, for example human. In a further aspect, the cell has been isolated from a subject prior to the contacting step. In a further aspect, contacting is via administration to a subject.

A. Treatment of a Disorder of Uncontrolled Cellular Proliferation

In one aspect, the invention relates to a method for the treatment of a disorder of uncontrolled cellular proliferation in a mammal, the method comprising the step of administering to the mammal an effective amount of least one disclosed compound or a product of a disclosed method of making a compound, or a pharmaceutically acceptable salt, hydrate, solvate, or polymorph thereof, thereby treating the disorder of uncontrolled cellular proliferation.

In a still further aspect, the effective amount is a therapeutically effective amount. In a yet still further aspect, the effective amount is a prophylactically effective amount.

In a further aspect, the mammal is a human. In a yet further aspect, the method further comprises the step of identifying a mammal in need of treatment of a disorder of uncontrolled cellular proliferation. In a still further aspect, the mammal has been diagnosed with a need for treatment of a disorder of uncontrolled cellular proliferation prior to the administering step.

In a further aspect, the disorder of uncontrolled cellular proliferation is associated with a histone demethylase dysfunction. In a further aspect, the histone demethylase is a lysine-specific histone demethylase. In a yet further aspect, the lysine-specific histone demethylase is LSD1. In an even further aspect, the lysine-specific histone demethylase is LSD2.

In a further aspect, the disorder of uncontrolled cellular proliferation is a cancer. In a yet further aspect, the cancer is a leukemia. In an even further aspect, the cancer is a sarcoma. In a still further aspect, the cancer is a solid tumor. In a yet further aspect, the cancer is a lymphoma. In an even further aspect, the cancer is selected from chronic lymphocytic leukemia, small lymphocytic lymphoma, B-cell non-Hodgkin lymphoma, and large B-cell lymphoma. In a still further aspect, the cancer is selected from cancers of the blood, brain, genitourinary tract, gastrointestinal tract, colon, rectum, breast, livery, kidney, lymphatic system, stomach, lung, pancreas, and skin. In a yet further aspect, the cancer is selected from a cancer of the lung and liver. In an even further aspect, the cancer is selected from a cancer of the breast, ovary, testes and prostate. In a still further aspect, the cancer is a cancer of the breast. In a yet further aspect, the cancer is a cancer of the ovary. In an even further aspect, the cancer is a cancer of the prostate. In a still further aspect, the cancer is a cancer of the testes.

B. Decreasing Histone Demethylase Activity

In one aspect, the invention relates to a method for decreasing histone demethylase activity in a mammal, the method comprising the step of administering to the mammal an effective amount of at least one disclosed compound or a product of a disclosed method of making a compound, or a pharmaceutically acceptable salt, hydrate, solvate, or polymorph thereof, or a pharmaceutically acceptable salt, hydrate, solvate, or polymorph thereof, thereby decreasing histone demethylase activity in the mammal.

In a still further aspect, the effective amount is a therapeutically effective amount. In a yet still further aspect, the effective amount is a prophylactically effective amount.

In a further aspect, the mammal is a human. In a yet further aspect, the method further comprises the step of identifying a mammal in need of decreasing histone demethylase activity. In a still further aspect, the mammal has been diagnosed with a need for decreasing histone demethylase activity prior to the administering step.

In a further aspect, the histone demethylase is a lysine-specific histone demethylase. In a yet further aspect, the lysine-specific histone demethylase is LSD1. In an even further aspect, the lysine-specific histone demethylase is LSD2.

In a further aspect, the need for decreasing histone demethylase activity is associated with a histone demethylase dysfunction. In a yet further aspect, the histone demethylase dysfunction is associated with a disorder of uncontrolled cellular proliferation. In a yet further aspect, the method further comprises the step of identifying a mammal in need of treating a disorder of uncontrolled cellular proliferation. In a still further aspect, the mammal has been diagnosed with a need for treating a disorder of uncontrolled cellular proliferation prior to the administering step.

In a still further aspect, the disorder of uncontrolled cellular proliferation is a cancer. In a yet further aspect, the cancer is a leukemia. In an even further aspect, the cancer is a sarcoma. In a still further aspect, the cancer is a solid tumor. In a yet further aspect, the cancer is a lymphoma. In an even further aspect, the cancer is selected from chronic lymphocytic leukemia, small lymphocytic lymphoma, B-cell non-Hodgkin lymphoma, and large B-cell lymphoma. In a still further aspect, the cancer is selected from cancers of the blood, brain, genitourinary tract, gastrointestinal tract, colon, rectum, breast, livery, kidney, lymphatic system, stomach, lung, pancreas, and skin. In a yet further aspect, the cancer is selected from a cancer of the lung and liver. In an even further aspect, the cancer is selected from a cancer of the breast, ovary, testes and prostate. In a still further aspect, the cancer is a cancer of the breast. In a yet further aspect, the cancer is a cancer of the ovary. In an even further aspect, the cancer is a cancer of the prostate. In a still further aspect, the cancer is a cancer of the testes.

C. Decreasing Histone Demethylase Activity in Cells

In one aspect, the invention relates to a method for decreasing histone demethylase activity in at least one cell, the method comprising the step of contacting the at least one cell with an effective amount of least one disclosed compound or a product of a disclosed method of making a compound, or a pharmaceutically acceptable salt, hydrate, solvate, or polymorph thereof, or a pharmaceutically acceptable salt, hydrate, solvate, or polymorph thereof, thereby decreasing histone demethylase activity in the cell.

In a still further aspect, the effective amount is a therapeutically effective amount. In a yet still further aspect, the effective amount is a prophylactically effective amount.

In a further aspect, the cell is mammalian. In a still further aspect, the cell is human. In a yet further aspect, contacting is via administration to a mammal. In a further aspect, the method further comprises the step of identifying the mammal as having a need of decreasing histone demethylase activity in a cell. In a still further aspect, the mammal has been diagnosed with a need for decreasing histone demethylase activity prior to the administering step.

In a further aspect, the histone demethylase is a lysine-specific histone demethylase. In a yet further aspect, the lysine-specific histone demethylase is LSD1. In an even further aspect, the lysine-specific histone demethylase is LSD2.

In a further aspect, the need for decreasing histone demethylase activity in a cell is associated with a disorder of uncontrolled cellular. In a still further aspect, the disorder of uncontrolled cellular proliferation is a cancer. In a yet further aspect, the cancer is a leukemia. In an even further aspect, the cancer is a sarcoma. In a still further aspect, the cancer is a solid tumor. In a yet further aspect, the cancer is a lymphoma. In an even further aspect, the cancer is selected from chronic lymphocytic leukemia, small lymphocytic lymphoma, B-cell non-Hodgkin lymphoma, and large B-cell lymphoma. In a still further aspect, the cancer is selected from cancers of the blood, brain, genitourinary tract, gastrointestinal tract, colon, rectum, breast, livery, kidney, lymphatic system, stomach, lung, pancreas, and skin. In a yet further aspect, the cancer is selected from a cancer of the lung and liver. In an even further aspect, the cancer is selected from a cancer of the breast, ovary, testes and prostate. In a still further aspect, the cancer is a cancer of the breast. In a yet further aspect, the cancer is a cancer of the ovary. In an even further aspect, the cancer is a cancer of the prostate. In a still further aspect, the cancer is a cancer of the testes.

2. Manufacture of a Medicament

In one aspect, the invention relates to a method for the manufacture of a medicament for inhibition of histone demethylase activity in a mammal comprising combining a therapeutically effective amount of a disclosed compound or product of a disclosed method with a pharmaceutically acceptable carrier or diluent.

### F. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of this invention are illustrated in the following Examples. Starting materials and the requisite intermediates are in some cases commercially available, or can be prepared according to literature procedures or as illustrated herein.

The following exemplary compounds of the invention were synthesized. The Examples are provided herein to illustrate the invention, and should not be construed as limiting the invention in any way. The Examples are typically depicted in free base form, according to the IUPAC naming convention. However, some of the Examples were obtained or isolated in salt form.

Some of the Examples were obtained as racemic mixtures of one or more enantiomers or diastereomers. The compounds may be separated by one skilled in the art to isolate individual enantiomers. Separation can be carried out by the coupling of a racemic mixture of compounds to an enantiomerically pure compound to form a diastereomeric mixture, followed by separation of the individual diastereomers by standard methods, such as fractional crystallization or chromatography. A racemic or diastereomeric mixture of the compounds can also be separated directly by chromatographic methods using chiral stationary phases.

1. General Chemical Materials and Methods

All analytical or anhydrous grade reagents were purchased from commercial sources and were used without further purification. Solvents were of analytical or anhydrous grade (Sigma-Aldrich). Specialty chemicals and building blocks obtained from several suppliers were of the highest offered purity (always ≧95%).

NMR spectroscopy was performed on a Varian Unity 400 instrument with a 5 mm broadband probe and using standard pulse sequences. Chemical shifts (δ) are reported in parts-per-million (ppm) downfield from solvent references. Coupling constants (J-values) are expressed in Hz.

Mass spectrometry was performed on a Finnigan LCQ Duo LCMS ion trap electrospray (ESI) mass spectrometer. All samples were analyzed by positive ESI-MS and the mass-to-charge ratio (m/z) of the protonated molecular ion is reported.

Microwave-assisted reactions were performed on a Biotage Initiator 2.5 at various powers.

Hydrogenation reactions were performed on a standard Parr hydrogenation apparatus.

Reactions were monitored either by HPLC or TLC. When monitored by TLC, reactions were analyzed on Baker flexible-backed plates coated with 200 μm of silica gel containing a fluorescent indicator. Preparative TLC was performed on 20 cm×20 cm Analtech Uniplates coated with a 1000 or 2000 μm silica gel layer containing a fluorescent (UV 254) indicator. Elution mixtures are reported as v:v. Spot visualization was achieved using UV light.

Flash chromatography was performed on a Teledyne Isco CombiFlash RF 200 using appropriately sized Redisep Rf Gold or Standard normal-phase silica or reversed-phase C-18 columns. Crude compounds were adsorbed on silica gel, 70-230 mesh 40 Å (for normal phase) or Celite 503 (for reversed-phase) and loaded into solid cartridges. Elution mixtures are reported as v:v.

2. Preparation of the Compounds of the Invention

Compounds of the invention can be made by the following experimental procedures:

### (E)-N′-(4-chloro-7-hydroxy-2,3-dihydro-1H-inden-1-ylidene)-3-((4-methylpiperazin-1-yl) sulfonyl)benzohydrazide (SP-10041)

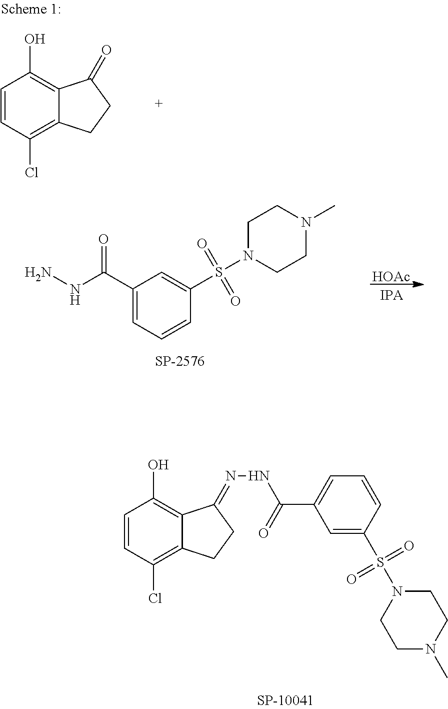 [SMILES: O=C1CCc2c(Cl)ccc(O)c21 ; CN1CCN(S(=O)(=O)c2cccc(C(=O)NN)c2)CC1 ; CN1CCN(S(=O)(=O)c2cccc(C(=O)N/N=C3\CCc4c(Cl)ccc(O)c43)c2)CC1 ; "HOAc"]

The reaction mixture of 4-chloro-7-hydroxy-2,3-dihydro-1H-inden-1-one (0.428 g, 2.346 mmol), 3-((4-methylpiperazin-1-yl)sulfonyl)benzohydrazide (0.7 g, 2.346 mmol) and acetic acid (0.537 mL, 9.38 mmol) in 20 mL of IPA was heated at 120° C. for 2 hr, Filtration, gave (E)-N′-(4-chloro-7-hydroxy-2,3-dihydro-1H-inden-1-ylidene)-3-((4-methylpiperazin-1-yl)sulfonyl)benzohydrazide (0.936 g, 2.022 mmol, 86% yield). ¹HNMR (400 MHz, CDCl₃) δ 8.88 (s, 1H), 8.142 (s, 1H), 8.05 (br-s, 1H), 7.90 (d, J=7.6 Hz, 1H), 7.66 (t, J=7.6 Hz, 1H), 6.78 (m, 1H), 3.19 (m, 2H), 3.07 (s, 4H), 2.94 (m, 2H), 2.47 (s, 4H), 2.26 (s, 3H). ESI-MS: 463.2 (M+H)⁺

### (E)-N′-(4-chloro-7-hydroxy-2,3-dihydro-1H-inden-1-ylidene)-3-(morpholinosulfonyl) benzohydrazide (SP-10042)

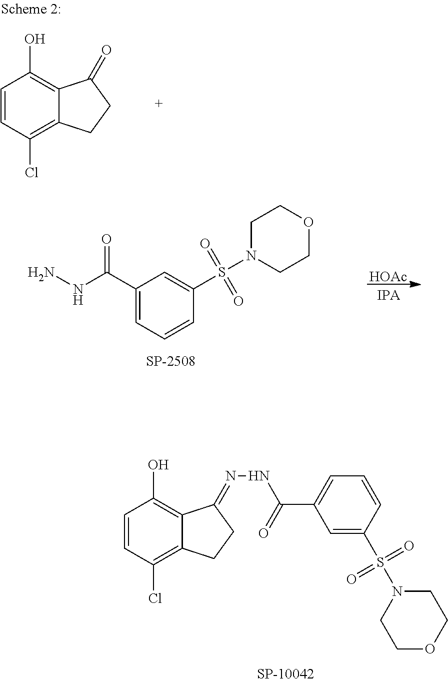 [SMILES: O=C1CCc2c(Cl)ccc(O)c21 ; NNC(=O)c1cccc(S(=O)(=O)N2CCOCC2)c1 ; O=C(N/N=C1\CCc2c(Cl)ccc(O)c21)c1cccc(S(=O)(=O)N2CCOCC2)c1 ; "HOAc"]

The reaction mixture of 4-chloro-7-hydroxy-2,3-dihydro-1H-inden-1-one (19.20 mg, 0.105 mmol), 3-(morpholinosulfonyl)benzohydrazide (30 mg, 0.105 mmol) and acetic acid (0.024 mL, 0.421 mmol) in 2-Propanol (2 mL) was heated at 120° C. for 2.45 hr, white precipitate formed overnight. Filtration and washed by MeOH gave (E)-N′-(4-chloro-7-hydroxy-2,3-dihydro-1H-inden-1-ylidene)-3-(morpholinosulfonyl) benzohydrazide (39 mg, 0.087 mmol, 82% yield). ¹HNMR (400 MHz, CDCl₃) δ 9.4 (s, 1H), 8.74 (s, 1H), 8.15 (s, 1H), 8.07 (s, 1H), 7.92 (t, J=7.6 Hz, 1H), 7.71 (t, J=7.6 Hz, 1H), 6.89 (s, 1H). ESI-MS: 450.3 (M+H)+.

### (E)-N′-(7-chloro-5-hydroxy-2,3-dihydro-1H-inden-1-ylidene)-3-((4-methylpiperazin-1-yl) sulfonyl)benzohydrazide (SP-10043)

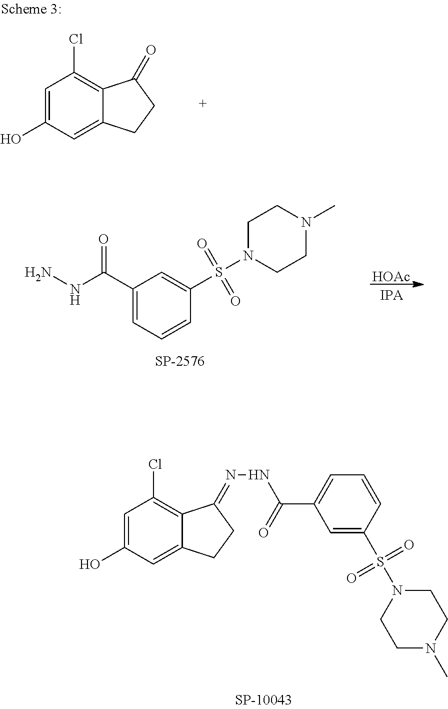 [SMILES: O=C1CCc2cc(O)cc(Cl)c21 ; CN1CCN(S(=O)(=O)c2cccc(C(=O)NN)c2)CC1 ; CN1CCN(S(=O)(=O)c2cccc(C(=O)N/N=C3\CCc4cc(O)cc(Cl)c43)c2)CC1 ; "HOAc"]

The reaction mixture of 7-chloro-5-hydroxy-2,3-dihydro-1H-inden-1-one (18.36 mg, 0.101 mmol), 3-((4-methylpiperazin-1-yl)sulfonyl)benzohydrazide (30 mg, 0.101 mmol), acetic acid (0.023 mL, 0.402 mmol) in 2-Propanol (1 mL) was heated at 120° C. for 2 hr, Concentration and purification by combiflash (DCM-MeOH) gave (E)-N′-(7-chloro-5-hydroxy-2,3-dihydro-1H-inden-1-ylidene)-3-((4-methylpiperazin-1-yl)sulfonyl) benzohydrazide (15 mg, 0.032 mmol, 32.2% yield). ¹HNMR (400 MHz, DMSO-d₆) δ 10.82 (s, 1H), 8.17 (m, 1H), 8.09 (s, 1H), 7.88 (s, 1H), 7.80 (m, 1H), 6.74 (d, J=8.0 Hz, 1H), 6.70 (m, 1H), 3.31 (s, 3H), 2.96 (s, 3H), 2.48 (s, 3H), 2.11 (s, 3H) ESI-MS:463.2 (M+H)+.

### (E)-N′-(4-fluoro-7-hydroxy-2,3-dihydro-1H-inden-1-ylidene)-3-((4-methylpiperazin-1-yl)sulfonyl)benzohydrazide (SP-10044)

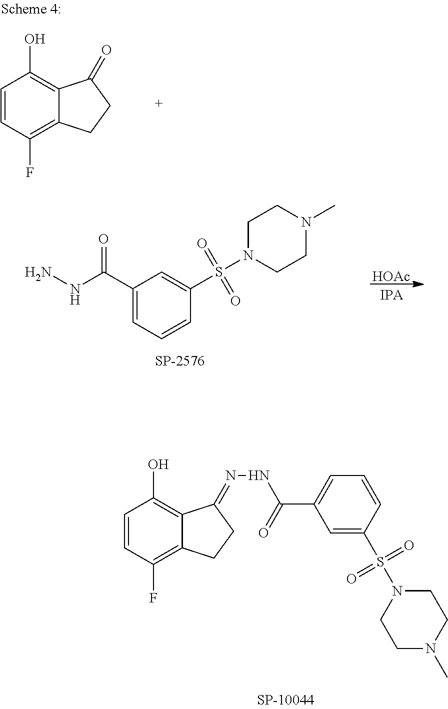 [SMILES: O=C1CCc2c(F)ccc(O)c21 ; CN1CCN(S(=O)(=O)c2cccc(C(=O)NN)c2)CC1 ; CN1CCN(S(=O)(=O)c2cccc(C(=O)N/N=C3\CCc4c(F)ccc(O)c43)c2)CC1 ; "HOAc"]

The reaction mixture of 4-fluoro-7-hydroxy-2,3-dihydro-1H-inden-1-one (16.71 mg, 0.101 mmol), 3-((4-methylpiperazin-1-yl)sulfonyl)benzohydrazide (30 mg, 0.101 mmol) and acetic acid (0.023 mL, 0.402 mmol) in 2-Propanol (1 mL) was heated at 120° C. for 2 hr, filtration gave (E)-N′-(4-fluoro-7-hydroxy-2,3-dihydro-1H-inden-1-ylidene)-3-((4-methylpiperazin-yl)sulfonyl) benzohydrazide (25 mg, 0.056 mmol, 55.7% yield). ¹HNMR (400 MHz, CD₃OD) δ 8.27 (s, 1H), 8.20 (d, J=8.4 Hz, 1H), 7.99 (d, J=8.0 Hz, 1H), 7.79 (t, J=7.6 Hz, 1H), 7.04 (t, J=8.4 Hz, 1H), 6.75 (m, 1H), 3.17 (s, 2H), 3.09 (s, 4H), 2.52 (s, 4H), 2.27 (s, 3H). ESI-MS: 448.1 (M+H)+.

### 1. 2-1. Preparation for Indanone 1

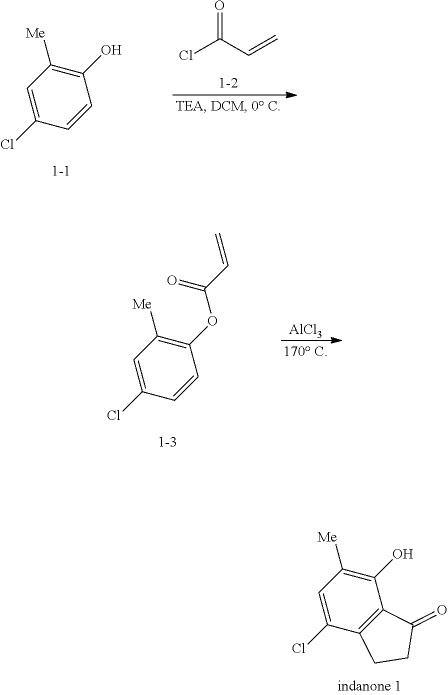

### 2.1-1. Compound 1-3: 4-chloro-2-methylphenyl acrylate

To a solution of compound 1-1 (10.0 g, 70.1 mmol, 1.0 eq) in DCM (100 mL) was added TEA (14.2 g, 140 mmol, 2.00 eq) and prop-2-enoyl chloride (7.00 g, 77.2 mmol, 1.10 eq). The mixture was stirred at 0° C. for 12 hour. The reaction mixture was diluted with DCM (100 mL), washed with water (30 mL*2) and brine (20 mL), dried over anhydrous sodium sulfate and filtered. The filtrate was concentrated under vacuum to give compound 1-3 as a colorless oil (11.5 g, 83% yield), which was used in the next step without further purification. ¹HNMR: (400 MHz, CDCl₃) δ 7.26 (d, J=2 Hz, 1H), 7.260-7.198 (m, 1H), 7.020 (d, J=8 Hz, 1H), 6.654 (d, J=17.2 Hz, 1H), 6.398-6.328 (m, 1H), 6.069 (d, J=9.6 Hz, 1H), 2.189 (s, 3H).

### 2.1-2. Indanone 1: 4-chloro-7-hydroxy-6-methyl-2,3-dihydro-1H-inden-1-one

Compound 1-3 (10.5 g, 53.4 mmol, 1.00 eq) was mixed with aluminum trichloride (71.2 g, 534 mmol, 10.0 eq) and was stirred at 170° C. for 1 hour. The mixture was cooled to 20° C. and diluted carefully with aq. HCl (4 N, 100 mL). Then the solution was extracted with EtOAc (100 mL*2), washed with con. NaHCO₃ (30 mL) and brine (50 mL), dried over anhydrous sodium sulfate, filtered and concentrated. The residue was purified by column chromatography (PE/EtOAc=100/1) first, then by prep-HPLC (0.1% TFA-ACN, Daiso 250*50 mm, 10 um) to afford Indanone 1 (2.40 g, 12.2 mmol, 23% yield) as a white solid. ¹HNMR: (400 MHz, CDCl₃) δ 9.128 (s, 1H), 7.301 (s, 1H), 3.044 (t, J=5.6 Hz, 2H), 2.743 (t, J=5.6 Hz, 2H), 2.225 (s, 3H)

### 2. 2-2. Preparation for Indanone 3

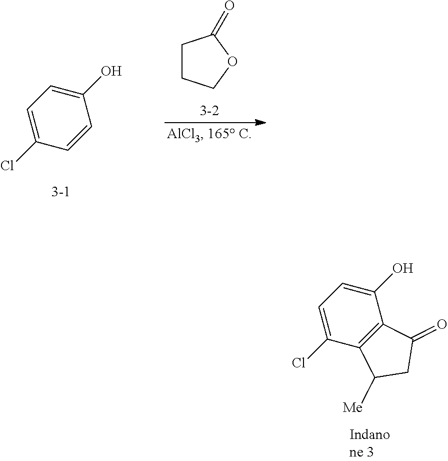 [SMILES: Oc1ccc(Cl)cc1 ; O=C1CCCO1 ; CC1CC(=O)c2c(O)ccc(Cl)c21]

### 2.2-1. Indanone 3: 4-chloro-7-hydroxy-3-methyl-2,3-dihydro-1H-inden-1-one

A mixture of compound 3-1 (20.0 g, 155 mmol, 1.00 eq), tetrahydrofuran-2-one (13.4 g, 155 mmol, 1.00 eq) and aluminium trichloride (207 g, 1.6 mol, 10.0 eq) was stirred at 165° C. for 1 hour. The mixture was cooled to 20° C. and diluted carefully with aq. HCl (4 N, 200 mL). Then the solution was extracted with EtOAc (250 mL*2), washed with con. NaHCO₃ (100 mL) and brine (100 mL), dried over anhydrous sodium sulfate, filtered and concentrated. The residue was purified by silica column chromatography (Petroleum ether/Ethyl acetate=100:1) to afford Indanone 3 as a yellow oil (9.00 g, 29% yield). ¹HNMR: (400 MHz, CDCl₃) δ 9.085 (s, 1H), 7.412 (d, J=8.8 Hz, 1H), 7.252 (d, J=8.8 Hz, 1H), 3.566-3.508 (m, 1H), 3.043-2.976 (m, 1H), 2.397-2.343 (m, 1H), 1.450 (d, J=7.2 Hz, 3H).

### 3. 2-3. Preparation for Indanone 4

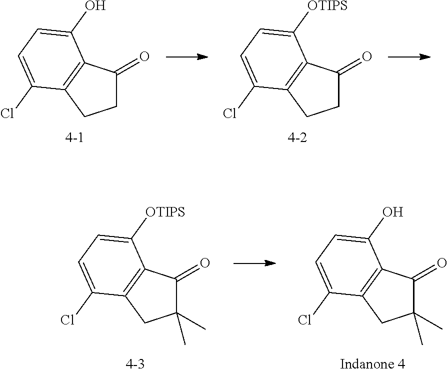

### Compound 4-2: 4-chloro-7-((triisopropylsilyl)oxy)-2,3-dihydro-H-inden-1-one

To a solution of compound 4-1 (2.00 g, 10.9 mmol, 1.00 eq) in DMF (20 mL) was added imidazole (894 mg, 13.1 mmol, 1.20 eq) and TIPSCI (2.22 g, 11.5 mmol, 1.05 eq). The mixture was stirred at 10° C. for 4 hr. The mixture was diluted with water (20 mL) and extracted with ethyl acetate (10 mL*3). The combined organic layer was dried over anhydrous sodium sulfate, filtered and concentrated. The crude was purified by column chromatography on silica gel (100-200 mesh, pure PE to PE:EtOAc=10:1) to give Compound 4-2 (3.70 g, 99% yield) as a yellow solid. ¹HNMR: (400 MHz, CDCl₃) δ 7.380 (d, J=8.0 Hz, 1H), 6.703 (d, J=8.0 Hz, 1H), 3.035 (t, J=6.0 Hz, 2H), 2.669 (t, J=6.0 Hz, 2H), 1.396-1.283 (m, 3H), 1.146 (d, J=3.6 Hz, 18H).

### Compound 4-3: 4-chloro-2,2-dimethyl-7-((triisopropylsilyl)oxy)-2,3-dihydro-1H-inden-1-one

To a solution of compound 4-2 (2.70 g, 8.00 mmol, 1.00 eq) in anhydrous THF (40 mL) was added NaH (797 mg, 19.9 mmol, 2.50 eq) under N₂ at 0° C. in portions. The reaction mixture was stirred at 0° C. for 0.5 hr. MeI (2.80 g, 20 mmol, 2.51 eq) was added to the above mixture through syringe under N₂ slowly. The mixture was stirred at 0° C. for 1 hr and then 15° C. for 0.5 hr. The mixture was quenched with water (2 mL) at 0° C. and stirred at 0° C. for about 15 minutes. The reaction mixture was used in next step directly without further work-up.

### Indanone 4: 4-chloro-7-hydroxy-2,2-dimethyl-2,3-dihydro-1H-inden-1-one

To a solution of compound 4-3 (3.30 g, 8.90 mmol, 1.00 eq) in THF (15 mL) was added TBAF3H₂O (5.60 g, 17.7 mmol, 2.00 eq). The mixture was stirred at 10° C. for 17 hours. The reaction mixture was diluted with EtOAc (150 mL) and washed with water (80 mL*2). The organic layer was dried over anhydrous Na₂SO₄, filtered and concentrated. The crude was purified by column chromatography first, eluting with PE/EtOAc=100/1 to 10/1, and then by pre-HPLC (Condition: 0.1% TFA-ACN, Begin B: 50,100% B, End B: 70, Hold Time (min), 4, Gradient Time (min): 30%-90%) to give Indanone 4 (680 mg, 36% yield) as a brown oil. ¹HNMR: (400 MHz, CDCl₃) δ 8.912 (s, 1H), 7.434 (d, J=8.8, 1H), 6.766 (d, J=8.8, 1H), 2.948 (s, 2H), 1.272 (s, 6H).

### 4. 2-4. Preparation for Indanone 5

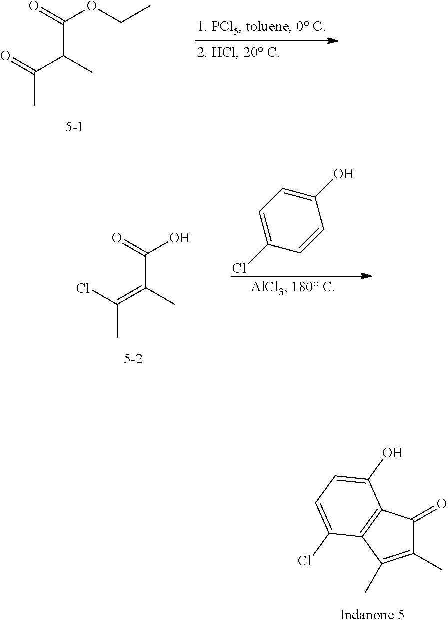

### Compound 5-2: (Z)-3-chloro-2-methylbut-2-enoic acid

To a solution of compound 5-1 (20.0 g, 138 mmol, 1.00 eq) in toluene (100 mL) was added pentachloro-phosphane (57.8 g, 277 mmol, 2.00 eq). The reaction mixture was stirred at 0° C. for 24 hour. Then water (50 mL) was added drop-wise and the mixture was stirred for an additional 24 hour. The reaction mixture was extracted with EtOAc (300 mL) and then washed with con NaHCO₃ aq (100 mL), brine (100 mL) and dried over anhydrous sodium sulfate. The solution was filtered and the filtrate was concentrated under vacuum to afford compound 5-2 as a colorless oil (1.60 g, crude product) which was used in the next step without further purification.

### Indanone 5: 4-chloro-7-hydroxy-2, 3-dimethyl-1H-inden-1-one

A mixture of 4-chlorophenol (1.00 g, 7.8 mmol, 1.00 eq), compound 5-2 (1.10 g crude, 7.8 mmol, 1.00 eq) and aluminium trichloride (10.4 g, 77.8 mmol, 10.00 eq) was stirred at 165° C. for 0.5 hr under N₂ atmosphere. The reaction mixture was cooled to 20° C., diluted carefully with aq HCl (4N, 5 mL) and extracted with DCM (30 mL). The organic layer was separated, dried over anhydrous sodium sulfate, filtered and concentrated. The crude product was purified by pre-HPLC (Column, Boston Green ODS 150*30 Su, Condition, 0.225% FA-ACN) to afford Indanone 5 as a yellow solid (20.0 mg, 1.2% yield). ¹HNMR: (400 MHz, CDCl₃) δ 8.101 (s, 1H), 6.990 (d, J=8.8, 1H), 6.569 (d, J=8.8, 1 H), 2.256 (s, 3H), 1.725 (s, 3H).

### 5. 2-5. Preparation for Indanone 9

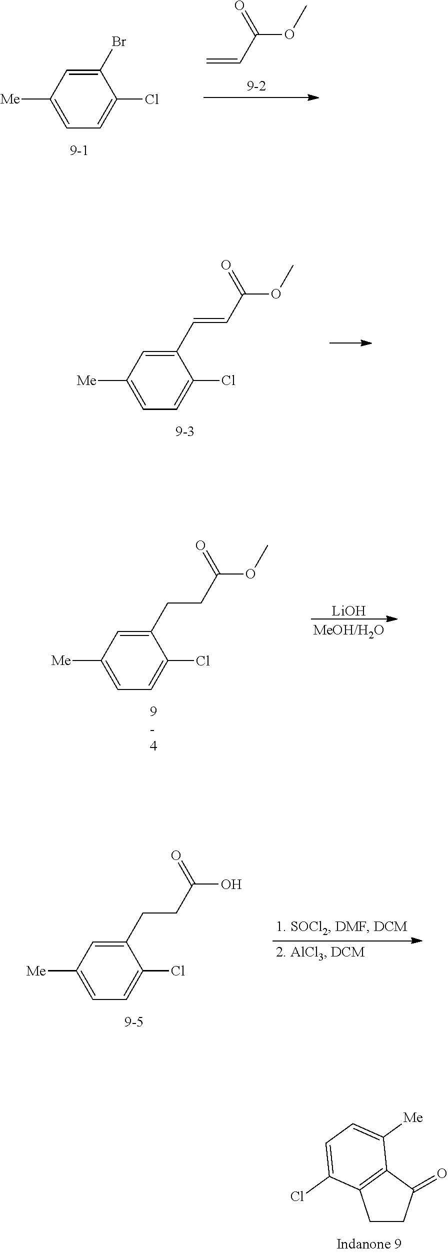

### Compound 9-3: (E)-methyl 3-(2-chloro-5-methylphenyl)acrylate

A suspension of compound 9-1 (10.0 g, 48.7 mmol, 1.00 eq) and compound 9-2 (12.6 g, 146 mmol, 3.01 eq), Pd₂(dba)₃ (2.20 g, 2.4 mmol, 0.05 eq), Cy₂NMe (28.5 g, 146 mmol, 3.00 eq) and tritert-butylphosphoniumtetrafluoroborate (847 mg, 2.9 mmol, 0.06 eq) in anhydrous dioxane (100 mL). After the reaction mixture was stirred at 75° C. for 13 hr, it was concentrated under reduced pressure to afford the crude product. The crude was then purified by column chromatography eluting with PE/EtOAc=100/1 to 10/1 to afford compound 9-3 as a white solid (2.70 g, 26% yield). ¹HNMR: (400 MHz, CDCl₃) δ 8.064 (d, J=16 Hz, 1H), 7.413 (s, 1H), 7.276 (t, J=8 Hz, 1H), 7.111 (d, J=8 Hz, 1H), 6.417 (d, J=16 Hz, 1H), 3.818 (s, 3H), 2.338 (s, 3H).

### Compound 9-4: methyl 3-(2-chloro-5-methylphenyl)propanoate

To a mixture of compound 9-3 (3.50 g, 15.0 mmol, 1.00 eq) in anhydrous MeOH (25 mL) was added NiCl₂ (5.80 g, 44.9 mmol, 3.00 eq), followed by NaBH₄ (1.70 g, 45.0 mmol, 3.01 eq) in portions under N₂. After addition, the solution turned black and was stirred at 20° C. for 1 hr. The reaction mixture was quenched with aq. NH₄Cl (30 mL) and filtered, the filter cake was washed with MeOH (50 mL). The filtrate was then concentrated under reduce pressure. The residue was diluted with EtOAc (60 mL*2) and washed with H₂O (50 mL). The organic layer was dried over anhydrous Na₂SO₄, filtered and concentrated under vacuum to give compound 9-4 (2.90 g, 56% yield) as colorless oil contained the de-chloride product. ¹HNMR: (400 MHz, CDCl₃) δ 7.260-7.181 (m, 2H), 7.054-6.972 (m, 4H), 3.684 (s, 3H), 3.676 (s, 2H), 3.037-2.998 (m, 2H), 2.917-2.896 (m, 2H), 2.657-2.617 (m, 3H), 2.328 (s, 2H), 2.293 (s, 3H).

### Compound 9-5: 3-(2-chloro-5-methylphenyl)propanoic acid

To a mixture of compound 9-4 (2.90 g, 8.30 mmol) in MeOH (20 mL) and H₂O (10 mL) was added LiOH.H₂O (1.40 g, 33.4 mmol, 4.00 eq). After addition the mixture was stirred at 20° C. for 16 hr. The solution was concentrated under reduced pressure. Then the mixture was diluted with H₂O, extracted with petroleum ether (120 mL*3). The aqueous layer was then acidified with HCl (1N) to pH=4. The solid precipitated and was collected by filtration. The filter cake was then washed with H₂O, dissolved with EtOAc, dried over Na₂SO₄, filtered to give compound 9-5 (2.50 g, crude) as a colorless oil which contained some des-chloro byproduct. ¹HNMR: (400 MHz, CDCl₃) δ 7.260-7.170 (m, 1H), 7.061-6.968 (m, 4H), 3.047-3.007 (m, 2H), 2.930-2.910 (m, 2H), 2.715-2.675 (m, 3H), 2.333 (s, 2H), 2.298 (s, 3H).

### Compound Indanone 9: 4-chloro-7-methyl-2, 3-dihydro-1H-inden-1-one

To a mixture of compound 9-5 (2.50 g, crude) in anhydrous DCM (25 mL) was added DMF (a few drops) and SOCl₂ (2.00 g, 16.6 mmol, 2.24 eq) slowly at 5° C. After the mixture was stirred at 15° C. for 0.5 hr, the solution was concentrated under reduced pressure to afford the crude product. Then the crude was dissolved with anhydrous DCM (25 mL) and cooled to 0° C. AlCl₃ (988 mg, 7.40 mmol, 1.00 eq) was added to the above mixture. The reaction mixture was then stirred at 10° C. for 16 hr. The reaction was quenched with aq. HCl (4 N, 15 mL), then extracted with DCM. The combined organic layer was washed with H₂O, dried over anhydrous Na₂SO₄, filtered and concentrated in vacuum to give the residue. The residue was then purified by pre-TLC (PE/EtOAc=2/1) to give Indanone 9 (900 mg, 67% yield for two steps) as a white solid. ¹HNMR: (400 MHz, CDCl₃) δ 7.416 (d, 1H), 7.072 (d, J=8 Hz, 1H), 3.066 (t, J=6.4 Hz, 2H), 2.705 (t, J=6.4 Hz, 2H), 2.606 (s, 3H).

### 6. 2-6. Preparation for Indanone 10

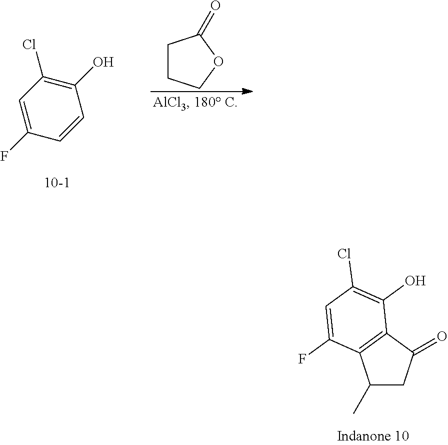

### Indanone 10: 6-chloro-4-fluoro-7-hydroxy-3-methyl-2,3-dihydro-1H-inden-1-one

A mixture of compound 10-1 (1.00 g, 6.80 mmol, 1.00 eq), tetrahydrofuran-2-one (587 mg, 6.80 mmol, 1.00 eq), AlCl₃ (9.10 g, 68.2 mmol, 10.0 eq) was stirred at 165° C. for 1 hr. The mixture was cooled to 20° C., diluted with HCl (4 N, 10 mL) carefully and extracted with EtOAc (25 mL*2). The combined organic layer was washed with con. NaHCO₃ (10 mL), brine (10 mL), dried over anhydrous sodium sulfate, filtered and concentrated in vacuum to give the crude product. The crude product was purified by silica gel column chromatography (Petroleum ether/Ethyl acetate-100:1) first and then pre-HPLC (Phenomenex Synergi C18 150*25*10 um, 0.225% FA-ACN) to give Indanone 10 (300 mg, 20% yield) as a white solid. ¹HNMR: (400 MHz, CDCl₃) δ 9.203 (br, 1H), 7.308 (d, J=8.4 Hz, 1H), 3.642-3.578 (m, 1H), 2.429-2.374 (m, 1H), 1.470 (d, J=6.8 Hz, 3H).

### 7. 2-7. Preparation for Indanone 11

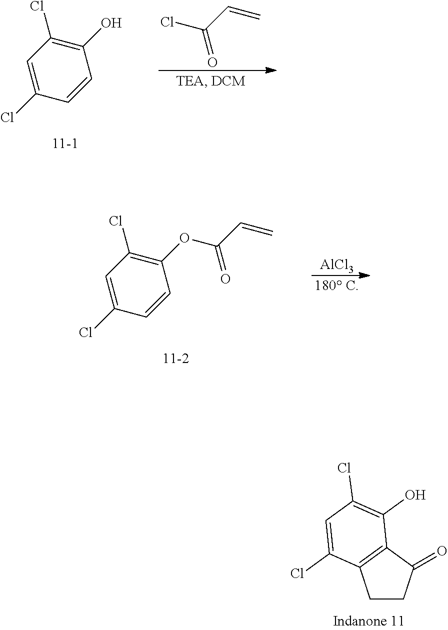

### Compound 11-2: 2, 4-dichlorophenyl acrylate

To a solution of compound 11-1 (5.00 g, 30.7 mmol, 1.00 eq) and TEA (6.20 g, 61.3 mmol, 2.00 eq) in DCM (50 mL) was added prop-2-enoyl chloride (3.30 g, 36.8 mmol, 1.20 eq). The mixture was stirred at 0° C. for 12 hr and then concentrated in vacuum. The residue was diluted with EtOAc, washed with aq. HCl (1 N, 20 mL) and brine (20 mL), dried over anhydrous sodium sulfate, filtered and concentrated to afford compound 11-2 (5.30 g, 24.4 mmol, 79.6% yield) as a yellow oil, which was used into the next step without further purification. ¹HNMR: (400 MHz, CDCl₃) δ 7.384 (s, 1H), 7.200-7.172 (m, 1H), 7.046 (d, J=8.8 Hz, 1H), 7.581 (d, J=17.2 Hz, 1H), 6.296-6.227 (m, 1H), 5.996 (d, J=10.8 Hz, 1H).

### Indanone 11: 4,6-dichloro-7-hydroxy-2,3-dihydro-1H-inden-1-one

A mixture of compound 11-2 (4.00 g, 18.4 mmol, 1.00 eq) and AlCl₃ (24.5 g, 184 mmol, 10.0 eq) was stirred at 180° C. for 0.5 hr. The mixture was cooled to 20° C. and diluted carefully with aq. HCl (4 N). Then the product was extracted with EtOAc, washed with con. NaHCO₃ and brine, dried over anhydrous sodium sulfate, filtered and concentrated to give the crude product. The crude was purified by silica column chromatography (Petroleum ether/Ethyl acetate=100/1) first and then by prep-HPLC (0.1% TFA-ACN, Daiso 250*50 mm, 10 um) to afford Indanone 11 (1.00 g, 25% yield) as a white solid. ¹HNMR: (400 MHz, CDCl₃) δ 7.462 (s, 1H), 3.011 (d, J=5.6 Hz, 1H), 2.738 (d, J=5.6 Hz, 1H).

### 8. 2-8. Preparation for Indanone 12

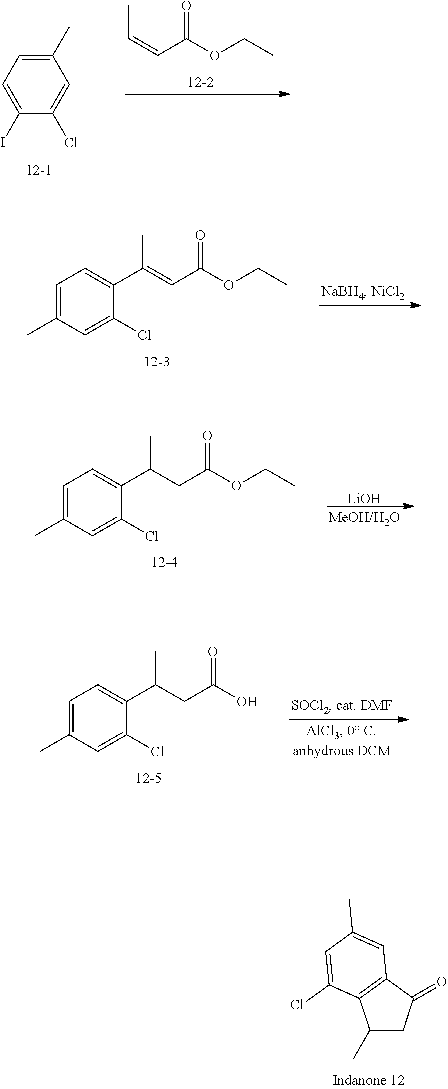

### Compound 12-3: (E)-ethyl 3-(2-chloro-4-methylphenyl)but-2-enoate

To a solution of compound 12-1 (10.0 g, 39.6 mmol, 1.00 eq) and compound 12-2 (6.30 g, 55.4 mmol, 1.40 eq) in MeCN (100 mL) was added Pd(OAc)₂ (450 mg, 2.00 mmol, 0.05 eq), TEA (10 g, 98.8 mmol, 2.49 eq) and tris-o-tolylphosphane (600 mg, 2 mmol, 0.05 eq) under N₂. The mixture was stirred at 100° C. under N₂ atmosphere for 14 hr and concentrated. The residue was purified by silica column chromatography (PE/EtOAc=50/1) to afford compound 12-3 (1.30 g, 12% yield) as a yellow oil. ¹HNMR: (400 MHz, CDCl₃) δ 7.203 (s, 1H), 7.048 (s, 2H), 5.810 (d, J=1.2 Hz, 1H), 4.239-4.185 (m, 2H), 2.473 (d, J=1.6 Hz, 3H), 2.331 (s, 3H), 1.309 (t, J=7.2 Hz, 3H).

### Compound 12-4: ethyl 3-(2-chloro-4-methylphenyl)butanoate

To a mixture of compound 12-3 (1.30 g, 4.90 mmol, 1.00 eq) in anhydrous MeOH (15 mL) was added NiCl₂ (635 mg, 4.90 mmol, 1.00 eq) and NaBH₄ (741 mg, 19.6 mmol, 4.00 eq) in portions at 10° C. under N₂. After addition, the reaction mixture turned black and stirred at 10° C. for 1 hr. The mixture was quenched with aq. NH₄Cl and filtered through a celite pad. The filter cake was washed with MeOH and the filtrate was concentrated under reduced pressure to remove MeOH. The residue was then diluted with EtOAc and washed with H₂O. The combined organic layer was dried over anhydrous Na₂SO₄, filtered and concentrated to give compound 12-4 (1.30 g, 98% yield) as a colorless oil. ¹HNMR: (400 MHz, CDCl₃) δ 7.173 (s, 1H), 7.136-7.108 (m, 1H), 7.040-7.020 (d, J=8.0 Hz, 1H), 4.13-4.07 (m, 2H), 3.78-3.73 (m, 1H), 2.689-2.635 (m, 1H), 2.53-2.49 (m, 1H), 2.29 (s, 3H), 1.270 (d, J=7.2 Hz, 3H), 1.190 (t, J=8.0 Hz, 3H).

### Compound 12-5: 3-(2-chloro-4-methylphenyl)butanoic acid

To a mixture of compound 12-4 (1.30 g, 4.40 mmol, 1.00 eq) in MeOH (16 mL) and H₂O (8 mL) was added LiOH (2.90 g, 121 mmol, 27.6 eq). The reaction mixture was stirred at 10° C. for 14 hr. The mixture was concentrated in vacuum. The remaining aqueous layer was then acidified with IM aq. HCl and extracted with EtOAc. The combined organic layer was dried over anhydrous Na₂SO₄, filtered and concentrated in vacuum to give compound 12-5 (800 mg, 77% yield) as a pale white solid. ¹HNMR: (400 MHz, CDCl₃) δ 7.184 (s, 1H), 7.127 (d, J=8.4 Hz, 1H), 7.040 (d, J=8.4 Hz, 1H), 3.756-3.735 (m, 1H), 2.759-2.705 (m, 1H), 2.562-2.501 (m, 1H), 2.296 (s, 3H), 1.302 (d, J=6.8 Hz, 3H).

### Compound Indanone 12: 4-chloro-3, 6-dimethyl-2,3-dihydro-H-inden-1-one

To a mixture of compound 12-5 (465 mg, 2.00 mmol, 1.00 eq) in anhydrous DCM (5 mL) was added DMF (144 ug, 2.00 umol, 1.00 eq) and SOCl₂ (656 mg, 5.50 mmol, 2.80 eq) at 10° C. After addition, the mixture was stirred at 10° C. for 0.5 hr. The solvent was evaporated to dryness. The residue was re-dissolved in anhydrous DCM (5 mL) and cooled to 0° C. AlCl₃ (265 mg, 2.00 mmol, 1.01 eq) was added to the above mixture and stirred at 20° C. slowly. After stirred at 20° C. for 16 hr, the reaction was quenched with 4 M aq. HCl. The mixture was extracted with DCM. The organic layer was washed with water, dried over Na₂SO₄, filtered and concentrated in vacuum to give the residue, which was purified by pre-TLC (PE/EA=2/1) to give indanone 12 (170 mg, 38% yield) as a pale yellow solid. ¹HNMR: (400 MHz, CDCl₃) δ 7.435 (s, 1H), 7.405 (s, 1H), 3.561-3.525 (m, 1H), 2.986-2.919 (m, 1H), 2.387 (s, 3H), 1.142 (d, J=6.8 Hz, 3H).

### 9. 2-9. Preparation for Indanone 13

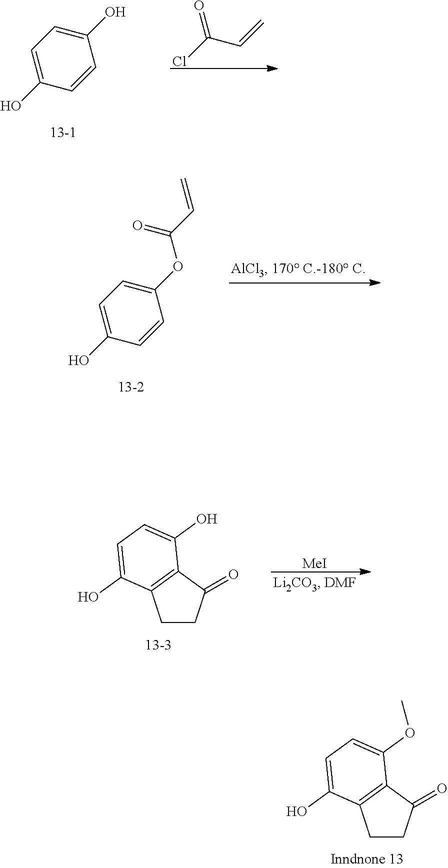

### Compound 13-2: 4-hydroxyphenyl acrylate

To a solution of compound 13-1 (20.0 g, 181 mmol, 1.00 eq) and TEA (36.8 g, 363 mmol, 2.00 eq) in anhydrous THF (300 mL) was added prop-2-enoyl chloride (16.5 g, 181 mmol, 1.00 eq) dropwise at −30° C. After addition, the mixture was stirred at 0° C. for 0.5 hr. The reaction was quenched with water at 0° C. and the product was extracted with EtOAc. The organic layer was separated and concentrated under vacuum. The residue was purified by column chromatography eluting with Petroleum ether/Ethyl acetate=30/1 to 20/1 to afford Compound 13-2 (12.3 g, 39% yield) as a pale yellow oil. ¹HNMR (400 MHz, CDCl₃) δ 6.951-6.929 (m, 2H), 6.754-6.732 (m, 2H), 6.030 (d, J=18.4 Hz, 1H), 6.015 (d, J=11.6 Hz, 1H), 6.00-5.73 (m, 1H), 5.728 (s, 1H).

### Compound 13-3: 4,7-dihydroxy-2,3-dihydro-1H-inden-1-one

A mixture of compound 13-2 (5.00 g, 28.9 mmol, 1.00 eq) and AlCl₃ (40.6 g, 304 mmol, 10.5 eq) was stirred at 170° C. for about 1.5 hr. The reaction was cooled to 20° C., quenched carefully with aq. HCl (4 N, 300 mL) and extracted with DCM. The combined organic layer was dried over anhydrous Na₂SO₄, filtered and concentrated. The residue was purified by column chromatography eluting with PE/EtOAc=30/1 to 10/1 to give Compound 13-3 (1.10 g, 20% yield) as a pale brown solid. ¹HNMR (400 MHz, DMSO-d₆) δ 9.125 (d, J=1.2 Hz, 2H), 6.896 (d, J=8.4 Hz, 1H), 6.58-6.55 (m, 1H), 2.87-2.84 (m, 2H), 2.57-5.50 (m, 2H).

### Indanone 13: 4-hydroxy-7-methoxy-2, 3-dihydro-1H-inden-1-one

To a mixture of compound 13-3 (940 mg, 4.90 mmol, 1.00 eq) in anhydrous DMF (10 mL) was added Li₂CO₃ (900 mg, 12.2 mmol, 2.50 eq) and MeI (3.50 g, 24.3 mmol, 1.51 mL, 4.99 eq). The reaction mixture was stirred at 50° C. for 16 hr. After the reaction was cooled to 20° C., the mixture was filtered and the filter cake was washed with DCM (20 mL). The filtrate was concentrated under reduced pressure to give the residue, which was purified by column chromatography eluting with PE/EtOAc=5/1 to 1/1 to give Indanone 13 (330 mg, 36.1% yield) as a brown solid. ¹HNMR (400 MHz, CDCl₃) δ 7.000 (d, J=8.4 Hz, 1H), 6.687 (d, J=8.4 Hz, 1H), 4.885 (s, 1H), 3.892 (s, 3H), 3.003 (t, J=5.4 Hz, 2H), 2.713 (t, 1=5.4 Hz, 2H).

### 10. 2-10. Preparation for Ketone 1

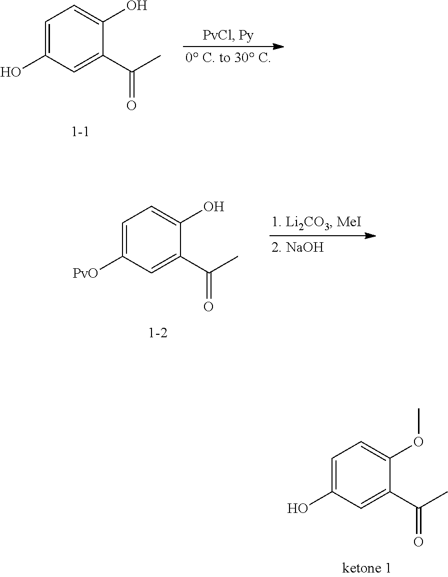

### Compound 1-2: 3-acetyl-4-hydroxyphenyl pivalate

2, 2-dimethylpropanoyl chloride (8.80 g, 73.0 mmol, 1.11 eq) was added drop-wise to a stirred solution of Compound 1-1 in pyridine (100 mL) at 0° C.-5° C. The mixture was stirred at 0° C. for 0.5 hr. The reaction mixture was diluted with DCM (100 mL) and washed with water (20 mL). The organic layer was dried over anhydrous sodium sulfate, filtered and concentrated in vacuum. The residue was purified by column chromatography eluting with PE/EtOAc=100/1 to give Compound 1-2 (2.10 g, 13% yield) as a colorless oil. ¹HNMR (400 MHz, CDCl₃) δ 12.130 (s, 1H), 7.406 (d, J=2.8 Hz, 1H), 7.183-7.154 (m, 1H), 6.979 (d, J=9.2 Hz, 1H), 2.617 (s, 3H), 1.363 (s, 9H)

### Ketone 1: 1-(5-hydroxy-2-methoxyphenyl)ethanone

To a solution of Compound 1-2 (2.20 g, 8.40 mmol, 1.00 eq) and Li₂CO₃ (9.90 g, 133 mmol, 15.9 eq) in anhydrous DMF (30 mL) was added MeI (4.20 g, 29.7 mmol, 3.54 eq). The reaction mixture was stirred at 100° C. for 26 hr. After cooled to 20° C., ten aqueous NaOH (2 M, 9 mL, 2.15 eq) was added. The mixture was stirred at 100° C. for 8 hr. The mixture was neutralized with aq. HCl (2 N, 250 mL). The product was extracted with EtOAc (300 mL*2), dried over anhydrous Na₂SO₄, filtered and concentrated to afford Ketone 1 (1.50 g, 96% yield) as a brown oil. ¹HNMR: (400 MHz, CDCl₃) δ 8.017 (s, 1H), 7.315 (d, J=3.2 Hz, 1H), 7.030-7.008 (m, 1H), 6.848 (d, J=9.2-1 Hz, 1H), 3.845 (s, 3H), 2.608 (s, 3H).

### 3. Preparation of Hydrazide Intermediates

### 11.3.1 Preparation for Intermediate 1, 2, 3, 4 and 7

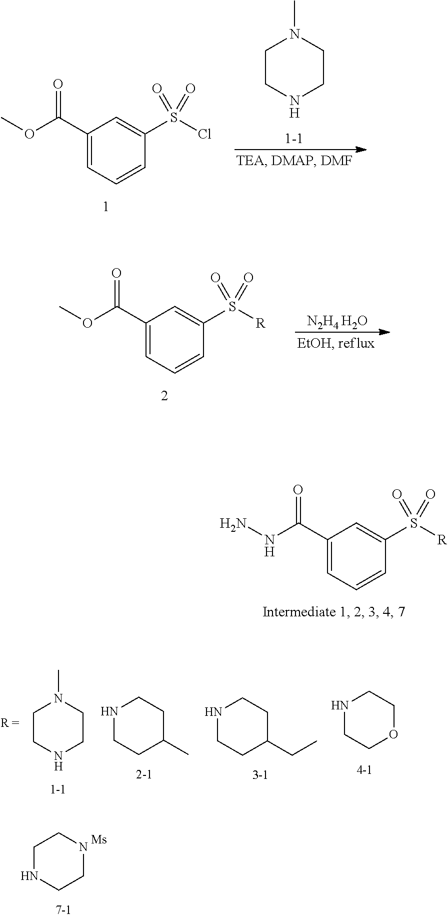

### Compound 1-2: methyl 3-((4-methylpiperazin-1-yl)sulfonyl)benzoate

To a solution of Compound 1 (3.30 g, 32.8 mmol, 1.10 eq) in DMF (70 mL) was added TEA (6.00 g, 59.6 mmol, 2.00 eq) and DMAP (182 mg, 1.50 mmol, 0.05 eq). Then Compound 1-1 (7.00 g, 29.8 mmol, 1.00 eq) was added drop-wise at 15° C. The mixture was stirred at 15° C. for 2 hr. The solution was diluted with water (70 mL) and extracted with EtOAc (100 mL*3). The combined organic layer was washed with aq. HCl (1N, 50 mL) and brine (50 mL), dried over anhydrous sodium sulfate, filtered and concentrated under vacuum to afford Compound 2 (8.00 g, 89% yield) as a colorless oil. ¹HNMR: (400 MHz, CDCl₃) δ 8.245 (d, J=8 Hz, 1H), 7.999 (s, 1H), 7.921 (d, J=8 Hz, 1H), 7.619 (t, J=8 Hz, 1H), 3.945 (s, 3H), 3.054 (s, 4H), 2.491-2.469 (m, 4H), 2.258 (s, 3H).

Compound 2-2, Compound 3-2, Compound 4-2, compound 7-2 were prepared following the same procedure as for Compound 1-2

### Compound 2-2: methyl 3-((4-methylpiperidin-1-yl)sulfonyl)benzoate

¹HNMR: (400 MHz, CDCl₃) δ 8.400 (s, 1H), 8.255 (d, J=8 Hz, 2H), 7.944 (d, J=8 Hz, 1H), 7.623 (t, J=8 Hz, 1H), 3.990 (s, 3H), 3.786 (d, J=11.2 Hz, 2H), 2.271 (t, J=11.2 Hz, 2H), 1.674 (d, J=11.2 Hz, 2H), 1.322-1.239 (m, 3H), 0.914 (d, J=5.2 Hz, 3H).

### Compound 3-2: methyl 3-((4-ethylpiperazin-1-yl)sulfonyl)benzoate

¹HNMR: (400 MHz, CDCl₃) δ 8.378 (s, 1H), 8.247-8.191 (m, 1H), 7.921 (d, J=7.6 Hz, 1H), 7.610 (t, J=8 Hz, 1H), 3.941 (s, 3H), 3.106 (m, 4H), 2.504 (t, J=4.8 Hz, 4H), 2.409-2.355 (m, 2H). 1.004 (t, J=7.2 Hz, 3H).

### Compound 4-2: methyl 3-(morpholinosulfonyl)benzoate

¹HNMR: (400 MHz, CDCl₃) δ 8.398 (s, 1H), 8.294 (d, J=8 Hz, 1H), 7.940 (d, =8 Hz, 1H), 7.660 (t, J=8 Hz, 1H), 3.967 (s, 3H), 3.748 (t, J=8.8 Hz, 4H), 3.023 (t, J=8.8 Hz, 4H).

Compound 7-2: The crude was used for the next step directly.

### Intermediate 1: 3-((4-methylpiperazin-1-yl)sulfonyl)benzohydrazide

To a solution of compound 1-2 (8.20 g, 27.5 mmol, 1.00 eq) in EtOH (80 mL) was added hydrazine hydrate (14.0 g, 275 mmol, 10.0 eq). The mixture was stirred at 80° C. for 12 hr. The reaction mixture was concentrated under vacuum to afford Intermediate 1 as a white solid (8.10 g, 27.2 mmol, 98% yield). ¹HNMR: (400 MHz, DMSO) δ 8.149-8.129 (m, 2H), 7.860 (d, J=8 Hz, 1H), 7.743 (t, J=8 Hz, 1H), 2.894-2.881 (m, 4H), 2.346-2.335 (m, 4H), 2.111 (s, 3H).

Intermediate 2, Intermediate 3, Intermediate 4 and Intermediate 7 were prepared following the same procedure as for Intermediate 1

### Intermediate 2: 3-((4-methylpiperidin-1-yl)sulfonyl)benzohydrazide

¹HNMR: (400 MHz, DMSO) δ 10.096 (s, 3H), 8.132-8.114 (m, 2H), 7.857 (d, J=8 Hz, 1H), 7.727 (t, J=8 Hz, 1H), 3.626 (d, J=11.6 Hz, 2H), 2.200 (t, J=10.8 Hz, 2H), 1.638 (d, J=12.0 Hz, 2H), 1.299-1.273 (m, 1H), 1.165-1.096 (m, 2H), 0.836 (d, J=6.8 Hz, 3H).

### Intermediate 3: 3-((4-ethylpiperazin-1-yl)sulfonyl)benzohydrazide

¹H NMR: (400 MHz, DMSO) δ 10.102 (s, 1H), 8.152-8.130 (m, 2H), 7.860 (d, J=7.6 Hz, 1H), 7.745 (t, J=8 Hz, 1H), 2.937-2.887 (m, 4H), 2.390 (m, 4H), 2.304-2.251 (m, 2H), 0.908 (t, J=7.2 Hz, 3H).

### Intermediate 4: methyl 3-(morpholinosulfonyl)benzoate

¹HNMR: (400 MHz, DMSO) δ 10.121 (s, 1H), 8.171-8.135 (m, 2H), 7.871 (d, J=8 Hz, 1H), 4.612 (br, 3H), 3.625 (t, J=4 Hz, 1H), 2.879 (t, J=4 Hz, 4H), 3.023 (t, J=8.8 Hz, 4H).

Intermediate 7: The crude product was used for the next step directly (no HNMR available).

### 12. 3.2 Preparation for Intermediate 5 and 6

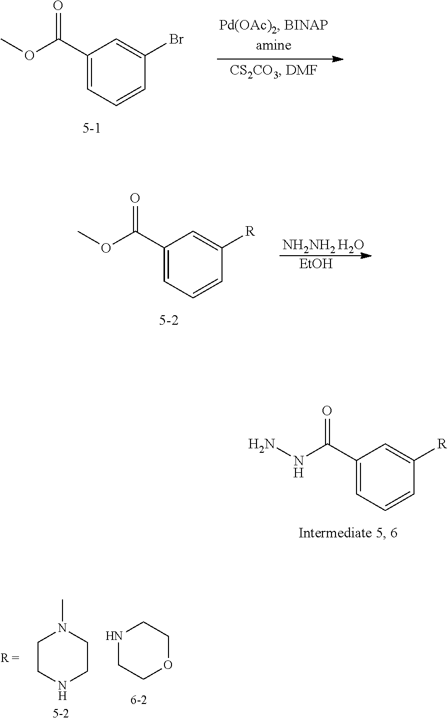

### Compound 6-2: methyl 3-(4-methylpiperazin-1-yl)benzoate

To a solution of Compound 5-1 (2.00 g, 9.30 mmol, 1.00 eq) and morpholine (972 mg, 11.2 mmol, 1.20 eq) in toluene (20 mL) was added Cs₂CO₃ (6.10 g, 18.6 mmol, 2.00 eq), BINAP (289 mg, 465 umol, 0.05 eq) and Pd(OAc)₂ (104 mg, 465 umol, 0.05 eq). The mixture stirred at 100° C. in N₂ atmosphere for 18 hr. The solid was filtered off and the filtrate was concentrated in vacuum. The residue was purified by column chromatography (PE:EtOAc=10:1 to 5:1) to afford Compound 6-2 (1.40 g, 69% yield) as a colorless oil. ¹HNMR: (400 MHz, CDCl₃) δ 7.607 (s, 1H), 7.567 (d, J=8 Hz, 1H), 7.357 (t, J=4 Hz, 1H), 7.134-7.128 (m, 4H), 3.930 (s, 3H), 3.897 (t, J=4.8 Hz, 4H), 3.230 (t, J=4.8 Hz, 4H).

Compound 5-2 was prepared following the same procedure as for Compound 6-2.

### Compound 5-2: methyl 3-morpholinobenzoate

¹HNMR: (400 MHz, CDCl₃) δ 8.403 (s, 1H), 8.295 (d, J=8 Hz, 1H), 7.944 (d, J=8 Hz, 1H), 7.660 (t, J=8 Hz, 1H), 3.971 (s, 3H), 3.750 (t, J=4.8 Hz, 4H), 3.029 (t, J=4.8 Hz, 4H). (The ¹H NMR was taken on the pilot batch.)

### Intermediate 6: 3-(4-methylpiperazin-1-yl)benzohydrazide

To a solution of Compound 6-2 (1.40 g, 6.40 mmol, 1.00 eq) in EtOH (15 mL) was added hydrazine hydrate (3.30 g, 63.7 mmol, 10.0 eq). The mixture was stirred at 80° C. for 14 hr. The solvent was removed in vacuum to give Intermediate 6 (1.70 g, crude) as a yellow solid. ¹HNMR: (400 MHz, DMSO) δ 9.706 (s, 1H), 7.356 (s, 1H), 7.316-7.245 (m, 2H), 77.090-7.066 (m, 1H), 3.747 (t, J=6.4 Hz, 4H), 3.142 (t, J=6.4 Hz, 4H).

Intermediate 5 was prepared following the same procedure as for Intermediate 6.

### Intermediate 5: 3-morpholinobenzohydrazide

¹HNMR: (400 MHz, DMSO) δ 9.685 (s, 1H), 7.348 (s, 1H), 7.285-7.231 (m, 2H), 7.067 (d, J=8 Hz, 1H), 3.174 (t, J=4.8 Hz, 4H), 2.458 (t, J=4.8 Hz, 4H), 2.227 (s, 1H).

### 4. Preparation of E Isomers

In this part, SP-10041_E was used to provide a general procedure for the preparation of E-isomers.

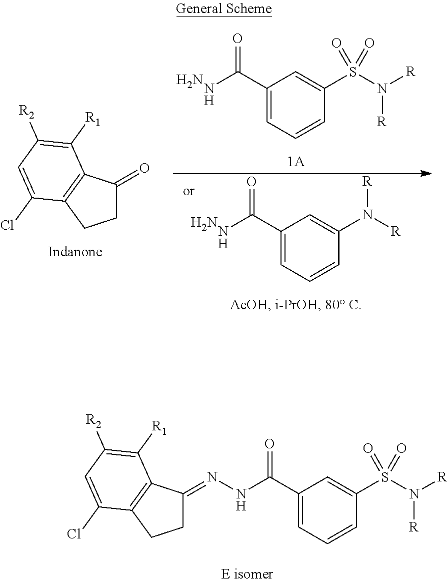

### General Procedure

### SP-10041_E: (E)-N′-(4-chloro-7-hydroxy-2,3-dihydro-1H-inden-1-ylidene)-3-((4-methylpiperazin-1-yl)sulfonyl)benzohydrazide

To a solution of Indanone 3 (4.00 g, 21.9 mmol, 1.00 eq) in isopropanol (80 mL) was added Intermediate 1 (6.50 g, 21.9 mmol, 1.00 eq) and acetate acid (5.30 g, 87.6 mmol, 4.00 eq). The mixture was stirred at 80° C. for 12 hr. The mixture was filtered. The filter cake was collected and dried under vacuum to afford SP-10041_E (5.20 g, 51% yield) as an off-white solid. ¹HNMR: (400 MHz, DMSO-d₆) δ 11.470 (s, 1H), 10.287 (s, 1H), 8.220 (d, J=8 Hz, 1H), 8.153 (s, 1H), 7.956 (d, J=8 Hz, 1H), 7.833 (t, J=8 Hz, 4H), 7.355 (d, J=8.8 Hz, 4H), 6.838 (d, J=8.4 Hz, 4H), 3.112 (m, 4H), 2.940 (m, 4H), 3.112 (m, 4H), 2.145 (s, 3H). LC-MS: [M+1]: 463.0

### SP-10046_E: (E)-N′-(4-chloro-7-hydroxy-6-methyl-2,3-dihydro-1H-inden-1-ylidene)-3-((4-methylpiperidin-1-yl)sulfonyl)benzohydrazide

¹H NMR: (400 MHz, DMSO) δ 11.472 (s, 1H), 10.459 (s, 1H), 8.206 (d, J=8 Hz, 1H), 8.158 (s, 1H), 7.951 (d, J=8.4 Hz, 1H), 7.835-7.797 (m, 1H), 7.280 (s, 1H), 3.655 (d, J=12.4 Hz, 2H), 3.092 (d, J=7.2 Hz, 4H), 2.334-2.235 (m, 1H), 2.201 (s, 3H), 1.663 (d, J=11.6 Hz, 2H), 1.153-1.133 (m, 3H), 0.857 (d, J=6.4 Hz, 3H). LC-MS: [M+1]: 476.2

### SP-10047_E: (E)-N′-(4-chloro-7-hydroxy-6-methyl-2,3-dihydro-1H-inden-1-ylidene)-3-((4-ethylpiperazin-1-yl)sulfonyl)benzohydrazide

¹HNMR: (400 MHz, DMSO-d₆) δ 11.500 (s, 1H), 10.456 (s, 1H), 8.228 (d, J=8 Hz, 1H), 8.151 (d, J=2.4 Hz, 1H), 7.956 (d, J=8 Hz, 1H), 7.856-7.817 (m, 1H), 7.282 (s, 1H), 3.090 (d, J=6.8 Hz, 4H), 2.930 (m, 4H), 2.422 (m, 4H), 2.311-2.275 (m, 2H), 2.201 (s, 3H), 0.924 (t, J=7.2 Hz, 3H). LC-MS [M+1]: 491.1

### SP-10048_E: (E)-N′-(4-chloro-7-hydroxy-6-methyl-2,3-dihydro-1H-inden-1-ylidene)-3-(morpholinosulfonyl) benzohydrazide

¹HNMR: (400 MHz, DMSO-d₆) δ 11.482 (s, 1H), 10.449 (s, 1H), 8.240 (d, J=7.6 Hz, 1H), 8.159 (s, 1H), 7.965 (d, J=7.6 Hz, 1H), 7.859 (d, J=7.6 Hz, 1H), 7.281 (s, 1H), 3.660-3.638 (m, 4H), 3.091 (d, J=6 Hz, 4H), 2.931-2.908 (m, 4H), 2.202 (s, 3H). LC-MS [M+H]: 464.1

### SP-10049_E: (E)-N′-(4-chloro-7-hydroxy-6-methyl-2,3-dihydro-1H-inden-1-ylidene)-3-(4-methylpiperazin-1-yl)benzohydrazide

¹HNMR: (400 MHz, DMSO) δ 11.126 (s, 1H), 10.529 (s, 1H), 8.190 (s, 1H), 7.361-7.340 (m, 2H), 7.290-7.247 (m, 2H), 7.180 (d, J=8.4 Hz, 1H), 3.220-3.164 (m, 6H), 3.088-3.055 (m, 6H), 7.280 (s, 1H), 3.655 (d, J=12.4 Hz, 2H), 3.092 (d, J=7.2 Hz, 4H), 2.334-2.235 (m, 1H), 2.246 (s, 3H), 2.080 (s, 3H). LC-MS: [M+1]: 413.2

### SP-10051_E: (E)-N′-(4-chloro-7-hydroxy-2,3-dihydro-1H-inden-1-ylidene)-3-(4-methylpiperazin-1-yl)benzohydrazide

¹HNMR: (400 MHz, DMSO) δ 7.490 (s, 1H), 7.406-7.386 (m, 2H), 7.284-7.179 (m, 2H), 6.770 (d, J=8.0 Hz, 1H), 3.330 (m, 4H), 3.136-3.109 (m, 4H), 2.703 (m, 4H), 2.417 (s, 3H). LC-MS: [M+1]: 399.2

### SP-10052_E: (E)-N-(4-chloro-7-hydroxy-2,3-dihydro-1H-inden-1-ylidene)-3-morpholinobenzohydrazide

¹HNMR: (400 MHz, DMSO) δ 7.403-7.305 (m, 4H), 7.190 (d, J=7.2 Hz, 1H), 6.827 (d, J=8.4 Hz, 2H), 3.774 (m, 4H), 3.192 (m, 4H), 3.103 (m, 3H). LC-MS: [M+1]: 386.1

### SP-10053_E: (E)-N′-(4-chloro-2,3-dihydro-1H-inden-1-ylidene)-3-((4-methylpiperazin-1-yl)sulfonyl)benzohydrazide

¹HNMR: (400 MHz, DMSO) δ 11.107 (s, 1H), 8.212-8.119 (m, 2H), 7.940 (d, J=8 Hz, 1H), 7.817 (t, J=8 Hz, 21H), 7.701 (d, J=7.2 Hz, 4H), 7.539 (d, J=7.2 Hz, 4H), 7.410 (m, 1H), 3.095-3.071 (m, 3H), 2.934 (m, 3H), 2.370 (m, 3H), 2.144 (m, 3H). LC-MS: [M+1]: 447.2

### SP-10055_E: (E)-N′-(4-chloro-7-hydroxy-3-methyl-2,3-dihydro-1H-inden-1-ylidene)-3-(morpholinosulfonyl)benzohydrazide

¹HNMR: (400 MHz, DMSO) δ 11.466 (s, 1H), 10.378 (s, 1H), 8.246 (d, J=8 Hz, 1H), 8.164 (s, 1H), 7.970 (d, J=8 Hz, 1H), 7.850 (t, J=8 Hz, 1H), 7.351 (d, J=8 Hz, 1H), 6.857 (d, 2H), 3.650 (m, 4H), 3.603-3.568 (m, 1H), 3.307-3.287 (m, 1H), 2.921 (m, 4H), 2.810 (d, J=18 Hz, 1H), 1.043 (d, J=8.8 Hz, 3H). LC-MS: [M+1]: 464.1

### SP-10056_E: (E)-N′-(6-chloro-4-fluoro-7-hydroxy-3-methyl-2,3-dihydro-1H-Inden-1-ylidene)-3-((4-methylpiperazin-1-yl)sulfonyl)benzohydrazide

¹HNMR: (400 MHz, DMSO) δ 8.395-8.336 (m, 2H), 8.036 (s, 1H), 7.817 (s, 1H), 7.195 (d, J=8.8 Hz, 1H), 3.622 (s, 1H), 3.120 (m, 4H), 2.751-2.680 (m, 2H), 2.589 (m, 4H), 2.326 (s, 3H), 1.422 (d, J=5.6 Hz, 3H). LC-MS: [M+1]: 495.1

### SP-10061_E: (E)-N′-(4-chloro-7-methyl-2,3-dihydro-1H-inden-1-ylidene)-3-((4-methylpiperazin-1-yl)sulfonyl)benzohydrazide

¹HNMR: (400 MHz, DMSO) δ 11.052-10.943 (m, 1H), 8.279-8.136 (m, 2H), 7.929-7.754 (m, 1H), 7.397-7.056 (m, 1H), 3.054 (s, 2H), 2.909 (m, 4H), 2.677-2.642 (m, 2H), 2.500 (s, 3H), 2.354 (m, 4H), 2.131 (s, 3H). LC-MS: [M+1]: 461.1

### SP-10065_E: (E)-N′-(4-chloro-3,6-dimethyl-2,3-dihydro-1H-inden-1-ylidene)-3-((4-methylpiperazin-1-yl)sulfonyl)benzohydrazide

¹HNMR: (400 MHz, DMSO) δ 10.978 (s, 1H), 10.128 (s, 1H), 8.368-8.137 (m, 3H), 7.931-7.821 (m, 2H), 7.520-7.364 (m, 1H), 3.372 (m, 2H), 2.918 (m, 4H), 2.746-2.685 (m, 6H), 2.362 (m, 7H), 2.135 (s, 2H), 1.300 (s, 3H). LC-MS: [M+1]: 475.1

### Analog 3_E: (E)-N′-(4-chloro-7-hydroxy-2,3-dihydro-1H-inden-1-ylidene)-3-((4-(methylsulfonyl)piperazin-1-yl)sulfonyl)benzohydrazide

¹HNMR: (400 MHz, DMSO) δ 8.240 (d, J=7.2 Hz, 1H), 8.173 (s, 1H), 7.964 (d, J=8 Hz, 1H), 7.843 (t, J=8 Hz, 1H), 7.350 (d, J=8 Hz, 1H), 3.224 (m, 4H), 3.111 (m, 4H), 3.058 (m, 4H), 2.902 (s, 3H). LC-MS: [M+1]: 527.0

### 5. Preparation of Z Isomers

In this part, SP-10041_Z was used to provide a general procedure for the preparation of Z isomers.

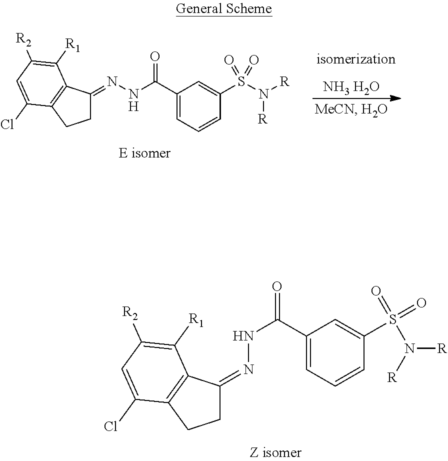

### SP-10041_Z: (E)-N′-(4-chloro-7-hydroxy-2,3-dihydro-1H-inden-1-ylidene)-3-((4-methylpiperazin-1-yl)sulfonyl)benzohydrazide

To a solution of SP-10041_E (5.00 g, 10.8 mmol, 1.00 eq) in MeCN (2500 mL) and water (250 mL) was added NH₃ H₂O (3.80 g, 108 mmol, 10.0 eq). The mixture was stirred at 20° C. for 24 hr. The mixture solution was concentrated under vacuum at 20° C. The residue was triturated with MeCN (100 mL) and EtOH (100 mL) to afford SP-10041_Z (1.50 g, 2.90 mmol, 27% yield) as a yellow solid. ¹HNMR: (400 MHz, DMSO) δ 8.224-8.161 (m, 2H), 7.972-7.758 (m, 2H), 7.370-7.228 (m, 1H), 6.854-6.551 (m, 1H), 3.117 (s, 1H), 3.065-2.890 (m, 7H), 2.572 (s, 3H), 2.550 (s, 1H), 2.277-2.212 (m, 3H). LC-MS: [M+1]: 463.0

### SP-10046_Z: (E)-N′-(4-chloro-7-hydroxy-6-methyl-2,3-dihydro-1H-inden-1-ylidene)-3-((4-methylpiperidin-1-yl)sulfonyl)benzohydrazide

¹HNMR: (400 MHz, DMSO) δ 8.434-8.155 (m, 2H), 1.941-7.724 (m, 2H), 7.281-6.976 (m, 1H), 3.718-3.641 (m, 2H), 3.100-3.081 (m, 2H), 2.899-2.665 (m, 2H), 2.331-2.104 (m, 4H), 1.824 (m, 2H), 1.301-1.130 (m, 4H), 0.849 (s, 3H). LC-MS: [M+1]: 476.1

### SP-10047_Z: (E)-N′-(4-chloro-7-hydroxy-6-methyl-2,3-dihydro-1H-Inden-1-ylidene)-3-((4-ethylpiperazin-1-yl)sulfonyl)benzohydrazide

¹HNMR: (400 MHz, DMSO) δ 8.348 (s, 1H), 8.149 (s, 1H), 7.786 (s, 1H), 7.544 (s, 1H), 7.194 (s, 1H), 3.031 (m, 7H), 2.552 (m, 4H), 2.453 (m, 2H), 2.149 (m, 4H), 0.942 (s, 3H). LC-MS: [M+1]: 491.2

### SP-10048_Z: (E)-N′-(4-chloro-7-hydroxy-6-methyl-2,3-dihydro-1H-inden-1-ylidene)-3-(morpholinosulfonyl)benzohydrazide

¹HNMR: (400 MHz, DMSO) δ 8.289 (s, 2H), 7.950-7.850 (m, 2H), 7.250 (s, 1H), 3.650 (m, 4H), 3.073 (m, 4H), 2.956 (m, 4H), 2.085 (s, 3H). LC-MS: [M+H]: 464.1

### SP-10049_Z: (E)-N′-(4-chloro-7-hydroxy-6-methyl-2,3-dihydro-1H-inden-1-ylidene)-3-(4-methylpiperazin-1-yl)benzohydrazide

¹HNMR: (400 MHz, DMSO) δ 11.127 (s, 1H), 7.449 (s, 1H), 7.430-7.327 (m, 2H), 7.307-7.260 (m, 2H), 7.091-7.051 (m, 1H), 3.092-3.067 (m, 4H), 2.890-2.782 (m, 8H), 2.400-2.197 (m, 4H), 2.013 (s, 3H). LC-MS: [M+1]: 413.2

### SP-10051_Z: (E)-N′-(4-chloro-7-hydroxy-2,3-dihydro-1H-inden-1-ylidene)-3-(4-methylpiperazin-1-yl)benzohydrazide

¹HNMR: (400 MHz, DMSO) δ 11.131 (s. 1H), 10.370 (s, 1H), 7.371-7.262 (m, 3H), 7.174-7.112 (m, 1H), 6.526-6.504 (m, 1H), 3.270 (m, 4H), 3.102 (m, 2H), 2.830 (m, 2H), 2.715-2.616 (m, 4H), 2.409-2.338 (m, 3H). LC-MS: [M+1]: 399.1

### SP-10052Z: (E)-N′-(4-chloro-7-hydroxy-2,3-dihydro-1H-inden-1-ylidene)-3-morpholinobenzohydrazide

¹HNMR: (400 MHz, DMSO) δ 7.692 (s, 1H), 7.568 (d, J=7.6 Hz, 1H), 7.051 (t, J=8 Hz, 1H), 6.902 (d, J=8 Hz, 1H), 6.145 (d, J=8 Hz. 1H), 3.772 (m, 4H), 3.192 (m, 4H), 2.718-2.681 (m, 4H). LC-MS: [M+1]: 386.1

### SP-10054_Z: (E)-N′-(4-chloro-7-hydroxy-3-methyl-2, 3-dihydro-1H-inden-1-ylidene)-3-((4-methylpiperazin-1-yl)sulfonyl)benzohydrazide

¹HNMR: (400 MHz, DMSO) δ 8.218-8.187 (m, 2H), 7.894-7.758 (m, 2H), 7.248 (d, J=8.8 Hz, 1H), 6.604 (d, J=8.8 Hz, 1H), 3.285-3.249 (m, 1H), 3.199-3.138 (m, 1H), 3.003 (m, 4H), 2.678 (m, 4H), 2.466 (m, 1H), 2.297 (s, 3H), 1.227 (d, J=6.8 Hz, 3H). LC-MS: [M+1]: 477.1

### SP-10055_Z: (E)-N′-(4-chloro-7-hydroxy-3-methyl-2,3-dihydro-1H-inden-1-ylidene)-3-(morpholinosulfonyl)benzohydrazide

¹HNMR: (400 MHz, DMSO) δ 8.214 (s, 1H), 7.866 (d, J=7.6 Hz, 1H), 7.710 (d, J=7.6 Hz, 1H), 7.231 (d, J=8.8 Hz, 1H), 6.596 (d, J=8.8 Hz, 1H), 3.632 (m, 4H), 3.284-3.248 (m, 1H), 3.191-3.131 (m, 1H). 2.912 (s, 3H), 2.461 (m, 1H), 1.227 (d, J=6.8 Hz, 3H). LC-MS: [M+1]: 464.1

### Analog 3_Z: (E)-N′-(4-chloro-7-hydroxy-2,3-dihydro-1H-inden-1-ylidene)-3-((4-(methylsulfonyl)piperazin-1-yl)sulfonyl)benzohydrazide

¹HNMR: (400 MHz, DMSO) δ 8.371 (d, J=7.6 Hz, 1H), 8.321 (s, 1H), 7.844 (d, J=7.6 Hz, 1H), 7.771 (s, 1H), 7.742 (d, J=8.0 Hz, 1H), 6.947 (d, J=8.0 Hz, 1H), 6.192 (d, J=8.8 Hz, 1H), 3.232 (m, 4H), 3.123 (m, 1H), 2.887 (s, 3H), 2.745 (m, 4H). LC-MS: [M+1]: 527.0

General Biochemical and Cell Materials and Methods

LSD1 activity was determined using a LSD1 Inhibitor Screening Assay Kit (Cayman Chemical Item Number 700120) purchased from Cayman Chemical Company (Ann Arbor, Mich.). Recombinant (expressed in baculovirus infected BTI insect cells) monoamine oxidase A and monoamine oxidase B (Catalog No. M7316 and M7441, respectively) were purchased from Sigma-Aldrich Co. LLC. (St. Louis, Mo.). MAO-Glo™ Assay Kit was purchased from Promega Corporation (Madison, Wis.). ATPlite™ Luminescence Assay System (e.g. Catalog No. V1401) was purchased from PerkinElmer Inc. (Waltham, Mass.).

Table 1 below lists some of the preferred compounds of the invention:

TABLE 1
(E/Z)-N′-(4-chloro-7-hydroxy-2,3-dihydro-1H-inden-1-ylidene)-3-substituted benzohydrazides.

| Num- | Reference | | Molecular | | Physical | |
|---|---|---|---|---|---|---|
| ber | Number | Structure | Weight | Purity | Property | Salt form |
| 1 | SP-10041_Z | 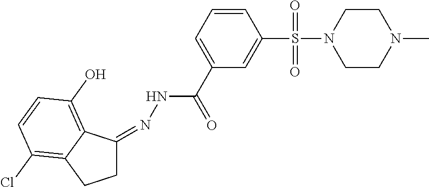 [SMILES: CN1CCN(S(=O)(=O)c2cccc(C(=O)N/N=C3/CCc4c(Cl)ccc(O)c43)c2)CC1] | 462.95 | 91.62% | Yellow Solid | free base |
| 2 | SP-10041_E | 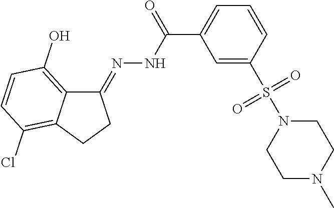 [SMILES: CN1CCN(S(=O)(=O)c2cccc(C(=O)N/N=C3\CCc4c(Cl)ccc(O)c43)c2)CC1] | 462.95 | 98% | Yellow Solid | free base |
| 3 | SP-10042_Z | 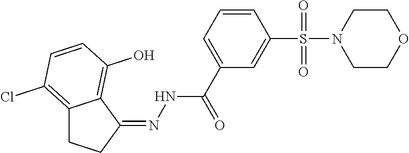 [SMILES: O=C(N/N=C1/CCc2c(Cl)ccc(O)c21)c1cccc(S(=O)(=O)N2CCOCC2)c1] | 449.91 | 94% | Yellow Solid | free base |
| 4 | SP-10042_E | 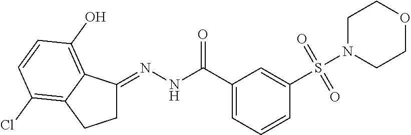 [SMILES: O=C(N/N=C1\CCc2c(Cl)ccc(O)c21)c1cccc(S(=O)(=O)N2CCOCC2)c1] | 449.91 | 95% | Yellow Solid | free base |
| 5 | SP-10043_Z | 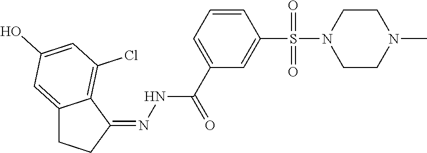 [SMILES: CN1CCN(S(=O)(=O)c2cccc(C(=O)N/N=C3/CCc4cc(O)cc(Cl)c43)c2)CC1] | 462.95 | 93% | Yellow Solid | free base |
| 6 | SF-10043-E | 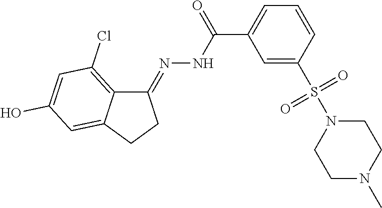 [SMILES: CN1CCN(S(=O)(=O)c2cccc(C(=O)N/N=C3\CCc4cc(O)cc(Cl)c43)c2)CC1] | 462.95 | 96% | Yellow Solid | free base |
| 7 | SP-10044_Z | 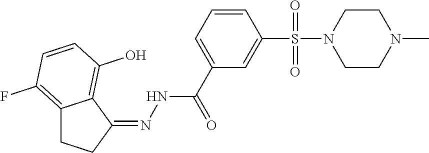 [SMILES: CN1CCN(S(=O)(=O)c2cccc(C(=O)N/N=C3/CCc4c(F)ccc(O)c43)c2)CC1] | 446.50 | 94% | Yellow Solid | free base |
| 8 | SP-10044_E | 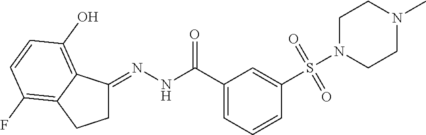 [SMILES: CN1CCN(S(=O)(=O)c2cccc(C(=O)N/N=C3\CCc4c(F)ccc(O)c43)c2)CC1] | 446.50 | 93% | Yellow Solid | free base |
| 9 | SP-10046_E | 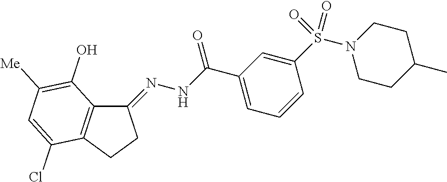 [SMILES: Cc1cc(Cl)c2c(c1O)/C(=N/NC(=O)c1cccc(S(=O)(=O)N3CCC(C)CC3)c1)CC2] | 475.99 | 99.64% | Light Yellow Solid | free base |
| 10 | SF-10048_E | 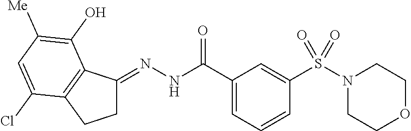 [SMILES: Cc1cc(Cl)c2c(c1O)/C(=N/NC(=O)c1cccc(S(=O)(=O)N3CCOCC3)c1)CC2] | 463.93 | 98.00% | Light Yellow Solid | free base |
| 11 | SP-10052_E | 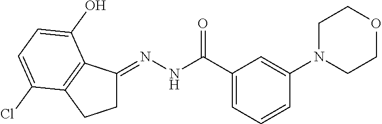 [SMILES: O=C(N/N=C1\CCc2c(Cl)ccc(O)c21)c1cccc(N2CCOCC2)c1] | 385.84 | 98.05% | White Solid | free base |
| 12 | SP-10055_E | 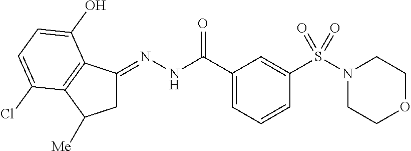 [SMILES: CC1C/C(=N\NC(=O)c2cccc(S(=O)(=O)N3CCOCC3)c2)c2c(O)ccc(Cl)c21] | 463.93 | 97.64% | White Solid | free base |
| 13 | SP-10055_Z | 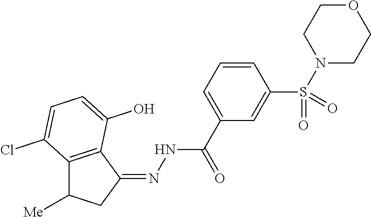 [SMILES: CC1C/C(=N/NC(=O)c2cccc(S(=O)(=O)N3CCOCC3)c2)c2c(O)ccc(Cl)c21] | 463.93 | 94.81% | White Solid | free base |
| 14 | SP-10047_E | 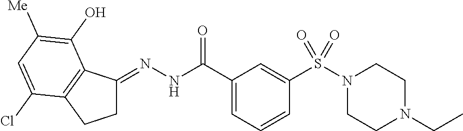 [SMILES: CCN1CCN(S(=O)(=O)c2cccc(C(=O)N/N=C3\CCc4c(Cl)cc(C)c(O)c43)c2)CC1] | 491.00 | 97.10% | Light Yellow Solid | free base |
| 15 | SP-10047_Z | 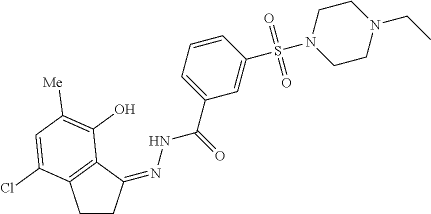 [SMILES: CCN1CCN(S(=O)(=O)c2cccc(C(=O)N/N=C3/CCc4c(Cl)cc(C)c(O)c43)c2)CC1] | 491.00 | 96.30% | Light Yellow Solid | free base |
| 16 | SP-10049_E | 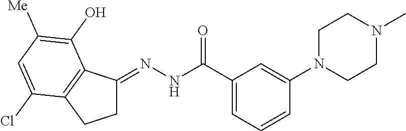 [SMILES: Cc1cc(Cl)c2c(c1O)/C(=N/NC(=O)c1cccc(N3CCN(C)CC3)c1)CC2] | 412.91 | 97.10% | Light Yellow Solid | free base |
| 17 | SP-10051_E | 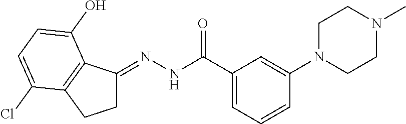 [SMILES: CN1CCN(c2cccc(C(=O)N/N=C3\CCc4c(Cl)ccc(O)c43)c2)CC1] | 398.89 | 95.88 | Light Yellow Solid | free base |
| 18 | SP-10053_E | 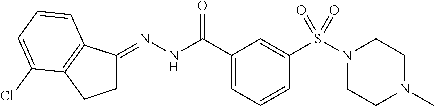 [SMILES: CN1CCN(S(=O)(=O)c2cccc(C(=O)N/N=C3\CCc4c(Cl)cccc43)c2)CC1] | 446.95 | 98.96% | Light Yellow Solid | free base |
| 19 | SP-10056_E | 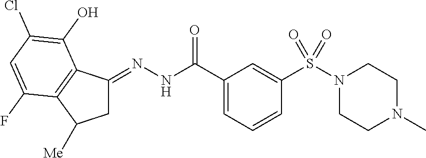 [SMILES: CC1C/C(=N\NC(=O)c2cccc(S(=O)(=O)N3CCN(C)CC3)c2)c2c(O)c(Cl)cc(F)c21] | 494.97 | 95.12% | Yellow Solid | free base |
| 20 | SP-10046_Z | 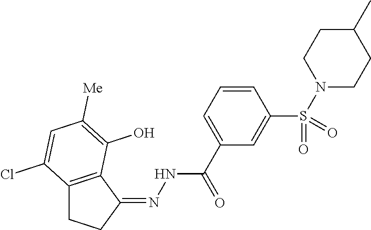 [SMILES: Cc1cc(Cl)c2c(c1O)/C(=N\NC(=O)c1cccc(S(=O)(=O)N3CCC(C)CC3)c1)CC2] | 495.99 | 94.66% | Light Yellow Solid | free base |
| 21 | SF-10048_Z | 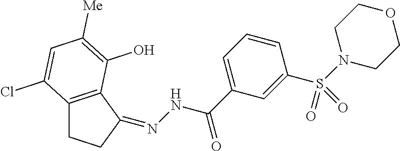 [SMILES: Cc1cc(Cl)c2c(c1O)/C(=N\NC(=O)c1cccc(S(=O)(=O)N3CCOCC3)c1)CC2] | 463.93 | 92.83% | Yellow Solid | free base |
| 22 | SP-10049_Z | 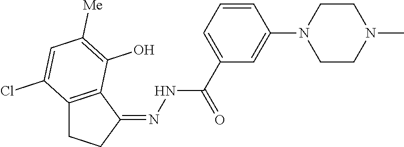 [SMILES: Cc1cc(Cl)c2c(c1O)/C(=N\NC(=O)c1cccc(N3CCN(C)CC3)c1)CC2] | 412.91 | 92.02% | Yellow Solid | free base |
| 23 | SF-10051_Z | 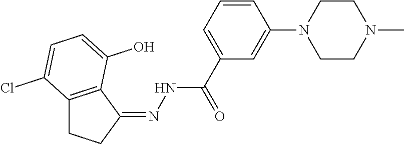 [SMILES: CN1CCN(c2cccc(C(=O)N/N=C3/CCc4c(Cl)ccc(O)c43)c2)CC1] | 398.89 | 99.73% | Yellow Solid | free base |
| 24 | SP-10061_E | 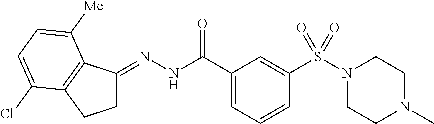 [SMILES: Cc1ccc(Cl)c2c1/C(=N/NC(=O)c1cccc(S(=O)(=O)N3CCN(C)CC3)c1)CC2] | 460.98 | 93.82% | White Solid | free base |
| 25 | SP-10065_E | 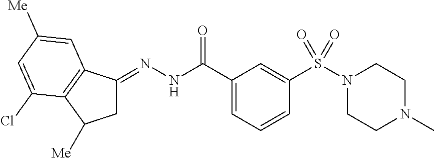 [SMILES: Cc1cc(Cl)c2c(c1)/C(=N/NC(=O)c1cccc(S(=O)(=O)N3CCN(C)CC3)c1)CC2C] | 475.00 | 98.60% | Yellow Solid | free base |
| 26 | SP-10052_Z | 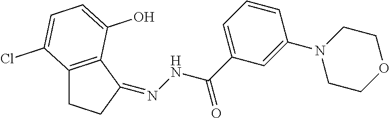 [SMILES: O=C(N/N=C1/CCc2c(Cl)ccc(O)c21)c1cccc(N2CCOCC2)c1] | 385.84 | 96.24% | Yellow Solid | free base |
| 27 | SP-10054_Z | 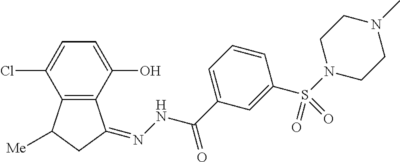 [SMILES: CC1C/C(=N/NC(=O)c2cccc(S(=O)(=O)N3CCN(C)CC3)c2)c2c(O)ccc(Cl)c21] | 476.98 | 97.85% | Yellow Solid | free base |
| 28 | SP-10054_E | 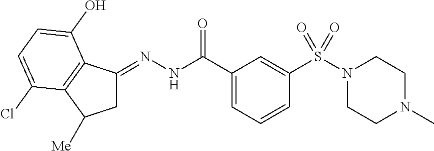 [SMILES: CC1C/C(=N\NC(=O)c2cccc(S(=O)(=O)N3CCN(C)CC3)c2)c2c(O)ccc(Cl)c21] | 476.98 | 97.26% | Yellow Solid | free base |
| 29 | SP-10055_Z | 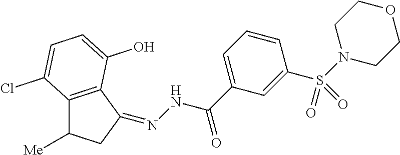 [SMILES: CC1C/C(=N/NC(=O)c2cccc(S(=O)(=O)N3CCOCC3)c2)c2c(O)ccc(Cl)c21] | 463.93 | 92.68% | Yellow Solid | free base |
| 30 | Analog 3_E | 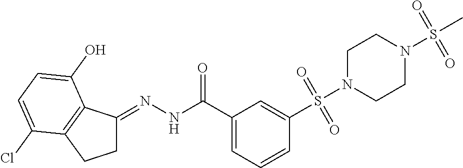 [SMILES: CS(=O)(=O)N1CCN(S(=O)(=O)c2cccc(C(=O)N/N=C3\CCc4c(Cl)ccc(O)c43)c2)CC1] | 527.01 | 97.40% | White Solid | free base |
| 31 | Analog 4 | 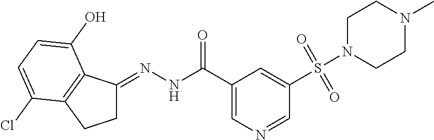 [SMILES: CN1CCN(S(=O)(=O)c2cncc(C(=O)N/N=C3\CCc4c(Cl)ccc(O)c43)c2)CC1] | 463.94 | 97.70% | Yellow Solid | free base |

LSD1 Histone Demethylase Assay

The primary assay for compound inhibitory activity was the LSD1 Inhibitor Screening Assay Kit (Cayman Chemical Company, Ann Arbor, Mich.; Cayman Chemical Item Number 700120). Briefly, test compounds were diluted to 20× the desired test concentration in 100% DMSO and 2.5 μL of the diluted test sample was added to a black 384-well plate. The LSD1 enzyme stock was diluted 17-fold with assay buffer, and 40 μL of the diluted LSD1 enzyme was added to the appropriate wells. Substrate, consisting of horseradish peroxidase, dimethyl K4 peptide corresponding to the first 21 amino acids of the N-terminal tail of histone H3, and 10-acetyl-3,7-dihydroxyphenoxazine was then added to wells. Resorufin was analyzed on an Envision plate reader with an excitation wavelength of 530 nm and an emission wavelength of 595 nm.

IC₅₀ Calculation

IC₅₀ values are determined using GraphPad Prism 5 software. The data were entered as an X-Y plot into the software as percent inhibition for each concentration of the drug. The concentration values of the drug were log transformed and the nonlinear regression was carried out using the “sigmoidal dose-response (variable slope)” option within the GraphPad software to model the data and calculate IC₅₀ values. The IC₅₀ values reported are the concentration of drug at which 50% inhibition was reached.

Compound Activity

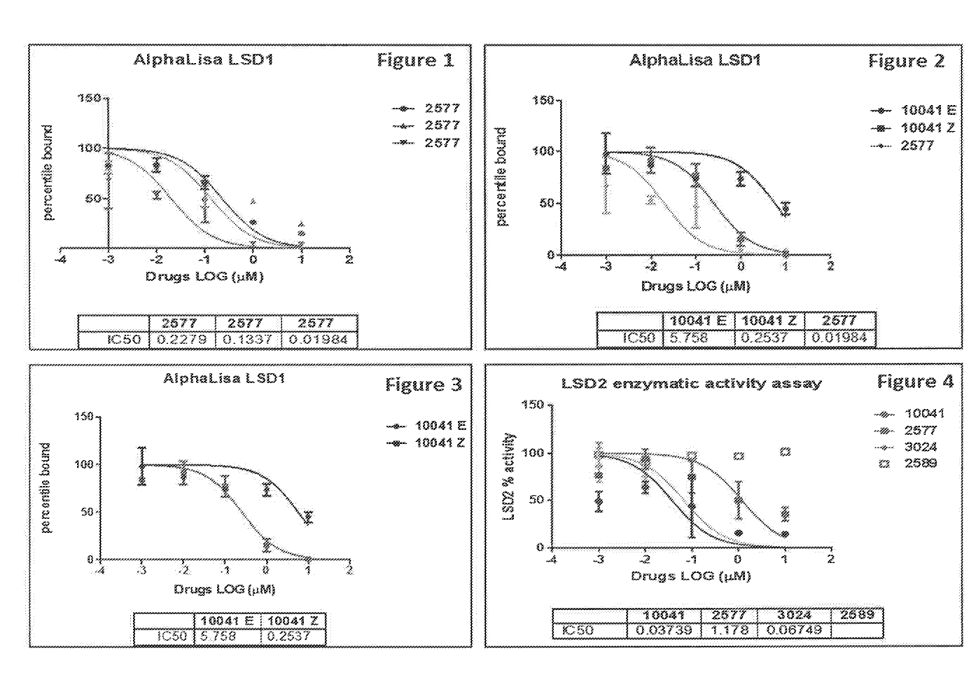

The ability of representative disclosed compounds to modulate various biochemical and cellular activities was determined using the assays described above. The results are shown in the tables below. The IC₅₀ (μM) for inhibition of both LSD1 and LSD2 activity shown in Table 2 and FIGS. 1-4. If an IC₅₀ or other assay result is indicated as “n.d.”, it was not determined in the indicated assay.

TABLE 2
E/Z)-N′-(4-chloro-7-hydroxy-2,3-dihydro-1H-inden-1-ylidene)-3-substituted
benzohydrazides series of LSD1, and LSD2 inhibition with an IC₅₀ in μM).

| | Reference | | LSD1 | Counter Assay |
|---|---|---|---|---|
| Number | Number | Structure | (IC₅₀ uM)* | (IC₅₀ uM)* |
| 1 | SP-10041_Z | 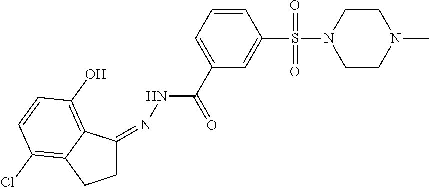 [SMILES: CN1CCN(S(=O)(=O)c2cccc(C(=O)N/N=C3/CCc4c(Cl)ccc(O)c43)c2)CC1] | 0.025 | NA |
| 2 | SP-10041_E | 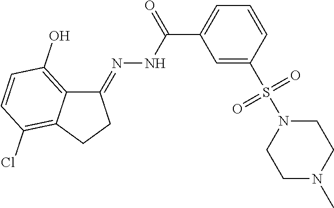 [SMILES: CN1CCN(S(=O)(=O)c2cccc(C(=O)N/N=C3\CCc4c(Cl)ccc(O)c43)c2)CC1] | 0.036 | NA |
| 3 | S4-10042_Z | 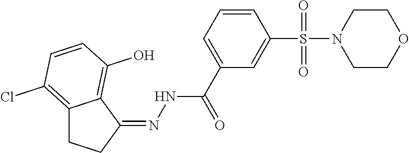 [SMILES: O=C(N/N=C1/CCc2c(Cl)ccc(O)c21)c1cccc(S(=O)(=O)N2CCOCC2)c1] | 0.498 | NA |
| 4 | SP-10042_E | 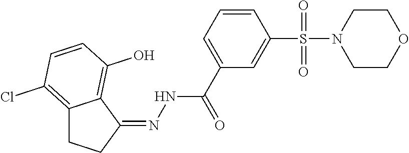 [SMILES: O=C(N/N=C1/CCc2c(Cl)ccc(O)c21)c1cccc(S(=O)(=O)N2CCOCC2)c1] | ND | NA |
| 5 | SP-10043_Z | 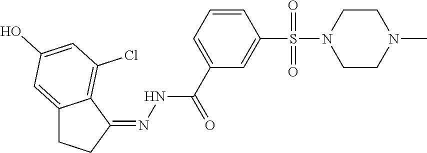 [SMILES: CN1CCN(S(=O)(=O)c2cccc(C(=O)N/N=C3/CCc4cc(O)cc(Cl)c43)c2)CC1] | >10 | NA |
| 6 | SP-10043-E | 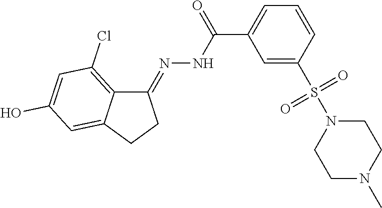 [SMILES: CN1CCN(S(=O)(=O)c2cccc(C(=O)N/N=C3\CCc4cc(O)cc(Cl)c43)c2)CC1] | >10 | NA |
| 7 | SP-10044_Z | 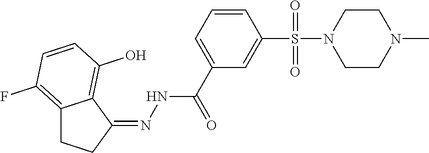 [SMILES: CN1CCN(S(=O)(=O)c2cccc(C(=O)N/N=C3/CCc4c(F)ccc(O)c43)c2)CC1] | 0.403 | NA |
| 8 | SP-10044_E | 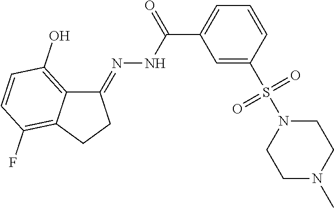 [SMILES: CN1CCN(S(=O)(=O)c2cccc(C(=O)N/N=C3\CCc4c(F)ccc(O)c43)c2)CC1] | ND | NA |
| 9 | SP-10046_E | 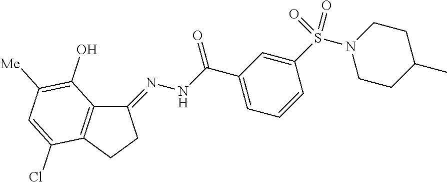 [SMILES: Cc1cc(Cl)c2c(c1O)/C(=N/NC(=O)c1cccc(S(=O)(=O)N3CCC(C)CC3)c1)CC2] | >10 | NA |
| 10 | SP-10048_E | 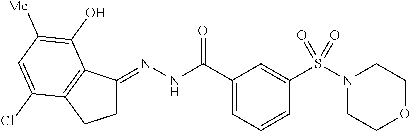 [SMILES: Cc1cc(Cl)c2c(c1O)/C(=N/NC(=O)c1cccc(S(=O)(=O)N3CCOCC3)c1)CC2] | NA | NA |
| 11 | SP-10052_E | 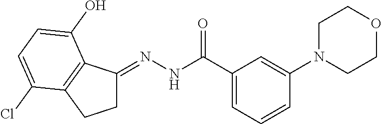 [SMILES: O=C(N/N=C1\CCc2c(Cl)ccc(O)c21)c1cccc(N2CCOCC2)c1] | NA | NA |
| 12 | SP-10055_E | 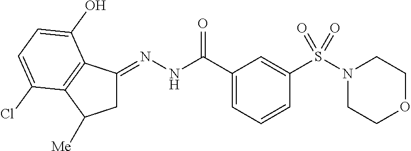 [SMILES: CC1C/C(=N\NC(=O)c2cccc(S(=O)(=O)N3CCOCC3)c2)c2c(O)ccc(Cl)c21] | 0.078 | NA |
| 13 | SP-10055_Z | 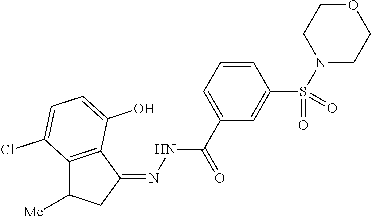 [SMILES: CC1C/C(=N/NC(=O)c2cccc(S(=O)(=O)N3CCOCC3)c2)c2c(O)ccc(Cl)c21] | 0.418 | NA |
| 14 | SP-10047_E | 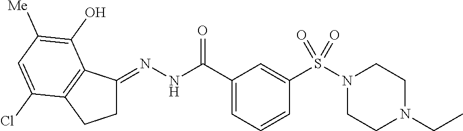 [SMILES: CCN1CCN(S(=O)(=O)c2cccc(C(=O)N/N=C3\CCc4c(Cl)cc(C)c(O)c43)c2)CC1] | NA | NA |
| 15 | SP-10047_Z | 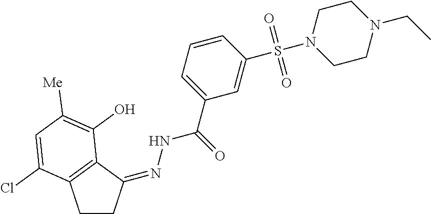 [SMILES: CCN1CCN(S(=O)(=O)c2cccc(C(=O)N/N=C3/CCc4c(Cl)cc(C)c(O)c43)c2)CC1] | 0.426 | NA |
| 16 | SP-10049_E | 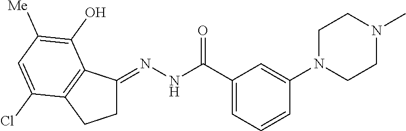 [SMILES: Cc1cc(Cl)c2c(c1O)/C(=N/NC(=O)c1cccc(N3CCN(C)CC3)c1)CC2] | NA | NA |
| 17 | SP-10051_E | 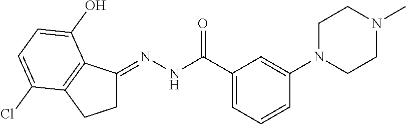 [SMILES: CN1CCN(c2cccc(C(=O)N/N=C3\CCc4c(Cl)ccc(O)c43)c2)CC1] | NA | NA |
| 18 | SP-10053_E | 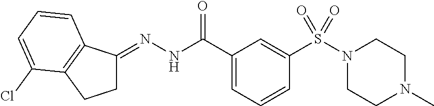 [SMILES: CN1CCN(S(=O)(=O)c2cccc(C(=O)N/N=C3\CCc4c(Cl)cccc43)c2)CC1] | NA | NA |
| 19 | SP-10056_E | 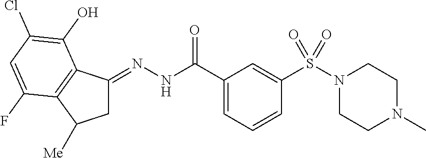 [SMILES: CC1C/C(=N\NC(=O)c2cccc(S(=O)(=O)N3CCN(C)CC3)c2)c2c(O)c(Cl)cc(F)c21] | ND | ND |
| 20 | SP-10046_Z | 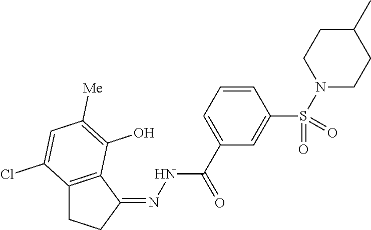 [SMILES: Cc1cc(Cl)c2c(c1O)/C(=N\NC(=O)c1cccc(S(=O)(=O)N3CCC(C)CC3)c1)CC2] | NA | NA |
| 21 | SP-10048_Z | 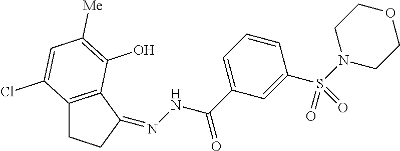 [SMILES: Cc1cc(Cl)c2c(c1O)/C(=N\NC(=O)c1cccc(S(=O)(=O)N3CCOCC3)c1)CC2] | NA | NA |
| 22 | SP-10049_Z | 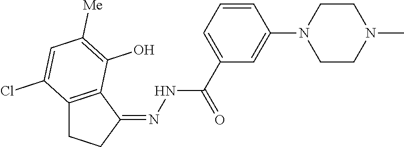 [SMILES: Cc1cc(Cl)c2c(c1O)/C(=N\NC(=O)c1cccc(N3CCN(C)CC3)c1)CC2] | NA | NA |
| 23 | SP-10051_Z | 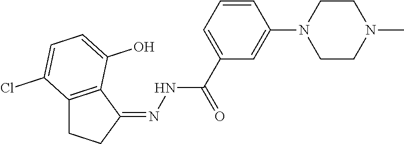 [SMILES: CN1CCN(c2cccc(C(=O)N/N=C3/CCc4c(Cl)ccc(O)c43)c2)CC1] | NA | NA |
| 24 | SP-10061_E | 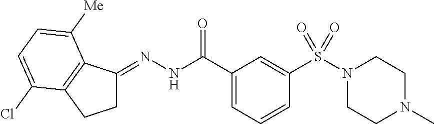 [SMILES: Cc1ccc(Cl)c2c1/C(=N/NC(=O)c1cccc(S(=O)(=O)N3CCN(C)CC3)c1)CC2] | 30.4 | NA |
| 25 | SP-10065_E | 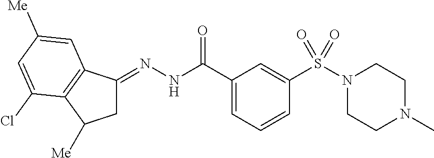 [SMILES: Cc1cc(Cl)c2c(c1)/C(=N/NC(=O)c1cccc(S(=O)(=O)N3CCN(C)CC3)c1)CC2C] | NA | NA |
| 26 | SP-10052_Z | 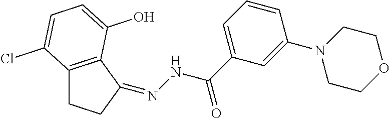 [SMILES: O=C(N/N=C1/CCc2c(Cl)ccc(O)c21)c1cccc(N2CCOCC2)c1] | NA | NA |
| 27 | SP-10054_Z | | 0.029 | NA |
| 28 | SP-10054_E | | ND | ND |
| 29 | SP-10055_Z | | ND | ND |
| 30 | Analog 3_E | | ND | ND |
| 31 | Analog 4 | | ND | ND |

*NA = Note Active, ND = Not Determined

LSD1 and LSD2 Enzymatic Activity of Selected Benzohydrazides

FIGS. 1-4 are the graphs depicting the LSD1 and LSD2 enzymatic activity of selected benzohydrazides. Specifically, FIG. 1 provides data for compound “2577” (not subject of the present application), which has the following chemical structure:

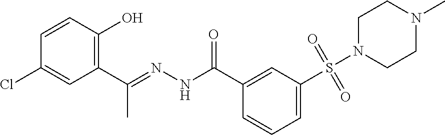 [SMILES: C/C(=N\NC(=O)c1cccc(S(=O)(=O)N2CCN(C)CC2)c1)c1cc(Cl)ccc1O]

FIGS. 2 and 3 provide data for compounds 2577 (FIG. 2 only), and also for compounds 10041E and 10041 Z, which structures are shown above in this application. FIG. 4 provides data for compounds 10041E, 2577, 3024 and 2589.

The “3024” compound has the following structure:

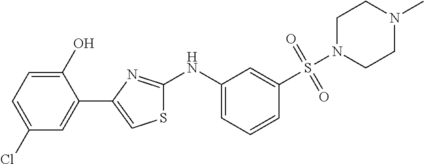 [SMILES: CN1CCN(S(=O)(=O)c2cccc(Nc3nc(-c4cc(Cl)ccc4O)cs3)c2)CC1]

The “2589” compound has the following structure:

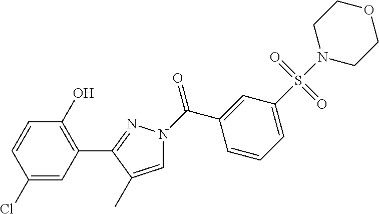 [SMILES: Cc1cn(C(=O)c2cccc(S(=O)(=O)N3CCOCC3)c2)nc1-c1cc(Cl)ccc1O]

These results show that the compounds of the invention, such as 10041E and 10041Z significantly inhibit LSD1 and LSD2 activity.

Cell Viability with Drug Screening

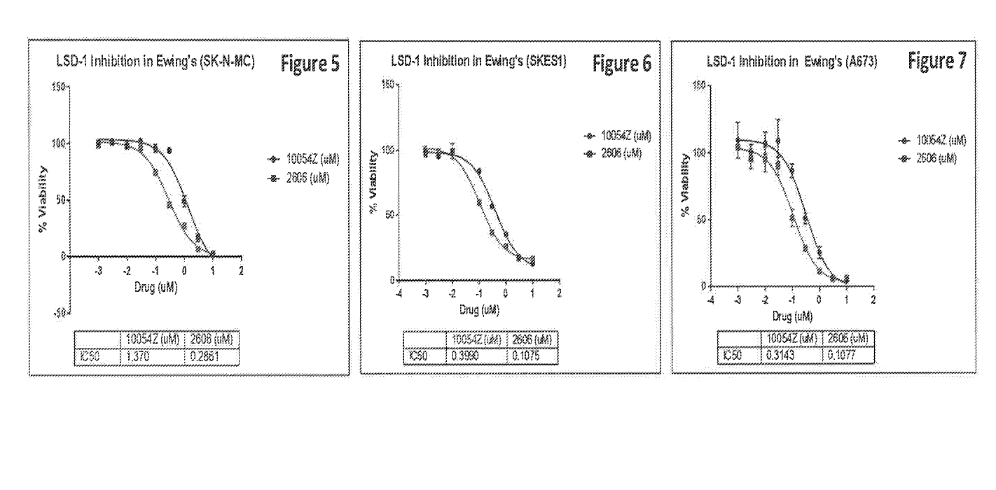

A hemocytometer count is performed to determine cell concentration. In a 96-well tissue culture plate, 2000 cells per well are plated for each drug concentration. Generally, 10 wells are done in duplicate. Then, the plates are placed in incubator overnight to allow cells to adhere to the plate (If suspension cells, proceed to the next step). Then, the desired drug concentrations are added to each well (ex. Serial dilutions 10 uM, 3 uM, 1 uM, 03 uM . . . etc.). Generally, each drug concentration is made at 10× in media and then 10 uL of drug is diluted in 90 uL of cells to get the final desired concentration. Then, the cells are incubated at 37° C. for 72 hours; after 72 hours, 100 uL of Promega Cell Titer Glo is added, and allowed incubation at room temperature for 10 minutes. Finally, the plates are read using the Envision software. Data analysis is performed using Graph Pad Prism 6. The results of the selected compounds on various cancer cells along with reference standards are provided in FIGS. 5-7.

FIGS. 5-7 depict graphs showing 72 hour time course cell viability of Ewing's sarcoma using CellTiter Glo (Promega). The tested compound was 10054Z and the control compound was 2606 which has the following structure:

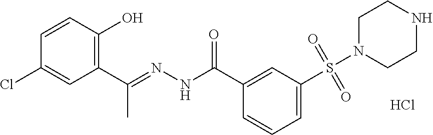 [SMILES: C/C(=N\NC(=O)c1cccc(S(=O)(=O)N2CCNCC2)c1)c1cc(Cl)ccc1O ; Cl]

The results show that 10054Z compound was able to significantly decrease the viability of Ewing's sarcoma cells.

Prophetic In Vivo Anti-Tumor Effects: Cell-Line Xenograft Model

The following examples of the in vive effect of the disclosed compounds are prophetic. Generally agents which modulate the regulation of chromatin, including histone demethylase inhibitors, display efficacy in preclinical models of cancer. In vivo effects of the compounds described in the preceding examples are expected to be shown in various animal models of cancer known to the skilled person, such as tumor xenograft models. These models are typically conducted in rodent, most often in mouse, but may be conducted in other animal species as is convenient to the study goals. Compounds, products, and compositions disclosed herein are expected to show in vivo effects in various animal models of cancer known to the skilled person, such as mouse tumor xenograft models.

In vivo effects of compounds can be assessed with in a mouse tumor xenograft study, one possible study protocol is described herein. Briefly, cells (2 to 5×10⁶ in 100 mL culture media) were implanted subcutaneously, e.g. by subcutaneous injection, in the right hind flank of athymic nu/nu nude mice (5 to 6 weeks old, 18-22 g). For test compounds of the present invention, a typical cell-line used for the tumor xenograft study would be AN3 CA or BT-20. Other suitable cell-lines for these studies are BT-549, HCT 116, HER218, MCF7, MDA-MB-231, MDA-MB-235, MDA-MB-435S, MDA-MB-468, PANC-1, PC-3, SK-N-MC, T-47D, and U-87 MG cells. The cells are cultured prior to harvesting for this protocol as described herein.

Following implantation, the tumors are allowed to grow to about 100 mm³, typically about 6-18 days post-implantation, before the animals are randomized into treatment groups (e.g. vehicle, positive control and various dose levels of the test compound); the number of animals per group is typically 8-12. Day 1 of study corresponds to the day that the animals receive their first dose. The efficacy of a test compound can be determined in studies of various lengths dependent upon the goals of the study. Typical study periods are for 14, 21 and 28-days. The dosing frequency (e.g. whether animals are dosed with test compound daily, every other day, every third day or other frequencies) is determined for each study depending upon the toxicity and potency of the test compound. A typical study design would involve dosing daily (M-F) with the test compound with recovery on the weekend. Throughout the study, tumor volumes and body weights are measured twice a week. At the end of the study the animals are euthanized and the tumors harvested and frozen for further analysis. Alternatively, tumors may be processed immediately for analysis, e.g. fixed in buffered-formalin, paraffin embedded, and sectioned for hematoxylin/eosin staining and further immunohistochemical analysis for desired oncology markers.

For example, compounds of the invention, or a pharmaceutically acceptable salt, solvate, polymorph, hydrate and the stereochemically isomeric form thereof, are expected to show such in vivo effects.

Prophetic In Vivo Anti-Tumor Effects: Tumor Graft Model

Alternatively, it can be desirable to assess the in vivo efficacy of the disclosed compounds in a tumor explant or tumor graft animal models (e.g. see Rubio-Viqueira B., et al. Clin Cancer Res. (2006) 12:4652-4661; Fiebig, H. H., Maier, A. and Burger, A. M. Eur. J. Canc. (2004) 40:802-820; and DeRose, Y. S., et al. “Patient-derived tumor grafts authentically reflect tumor pathology, growth, metastasis and disease outcomes.” (2011) Nat. Med., in press). These models can provide higher quality information on in vivo effects of therapeutic compounds. It is believed tumor graft models are more authentic in vivo models of many types of cancer, e.g. human breast cancer, with which to examine the biology of tumors and how they metastasize. Engraftment of actual patient tumor tissues into immunodeficient mice (termed ‘tumor grafts’) provides improvement over implantation of cell lines, in terms of phenocopying human tumors and predicting drug responses in patients (Clarke, R. Breast Cancer Res (2009) 11 Suppl 3, S22; Press, J. Z., et al. Gynecol Oncol (2008) 110:56-264; Kim, M. P., et al. Nat Protoc (2009) 4:670-1680; Daniel, V. C., et al. Cancer Res (2009) 69:3364-3373; and Ding, L., et al. Nature (2010) 464:999-1005).

Briefly, tissue samples will be collected from informed, consented patients at Huntsman Cancer Hospital/University of Utah under an approved IRB protocol. Samples will be collected and de-identified by the Huntsman Cancer Institute Tissue Resource and Application Core facility before being obtained for implantation. It is anticipated that all primary tumors will be from individuals that had not received chemotherapy prior to tissue collection, and and all metastatic effusions will be from individuals that had been treated with chemotherapy, hormone therapy, and/or radiation therapy. The University of Utah Institutional Animal Care and Use Committee will review and approve all mouse experiments. It is anticipated that a minimum of three mice per experimental group will be used, and only female mice will be used for studies involving breast cancer tumors. A single fragment of fresh or frozen tumor (˜8 mm3), or about 10⁶ cells in matrigel, is implanted into cleared inguinal mammary fat pads of 3-4 week old female NOD/SCID mice. At the same, interscapular estrogen pellets are subcutaneously implanted in mice with ER+ tumors. Tumor growth is measured weekly using calipers. When tumors reach about 150-2,000 mm³, the mice are euthanized, and tissue fragments are re-transplanted into another cohort of mice, frozen for later use, and/or analyzed for histology, gene expression, and DNA copy number. Tumor volumes are calculated using the formula 0.5×length×(width)². For experiments to determine estrogen dependence, ER* tumors are implanted into mice as described above, in the presence or absence of intrascapular estrogen pellets and with or without a concurrent surgical procedure to remove the ovaries, which is performed according to standard methods.

Freshly harvested tumor tissues from patients or mice are cut into ˜8 mm3 pieces and stored in liquid nitrogen, in a solution of 95% FBS and 5% DMSO for later implantation. Alternatively, the tissue is digested with collagenase solution (1 mg/ml collagenase [Type IV, Sigma] in RPMI 1640 supplemented with 2.5% FBS, 10 mM HEPES, 10 pg/mL penicillin-streptomycin) at 37° C. for 40-60 min, while shaking at 250 rpm. Digested tissue is strained to remove debris and washed in human breast epithelial cell (HBEC) medium (DMEM F/12 supplemented with 10 mM HEPES, 5% FBS, 1 mg/mL BSA, 0.5 μg/mL hydrocortisone, 50 μg mL Gentamycin, 1 μg/mL ITS-X100) three times. The pellet is resuspended in freezing medium (5% FBS and 10% DMSO in HBEC medium) and stored in liquid nitrogen.

To assess the effect of a disclosed compound, tumors in mice are allowed to grow to about 100 mm³, typically about 6-18 days post-implantation, before the animals are randomized into treatment groups (e.g. vehicle, positive control and various dose levels of the test compound); the number of animals per group is typically 8-12. Day 1 of study corresponds to the day that the animals receive their first dose. The efficacy of a test compound can be determined in studies of various lengths dependent upon the goals of the study. Typical study periods are for 14, 21 and 28-days. The dosing frequency (e.g. whether animals are dosed with test compound daily, every other day, every third day or other frequencies) is determined for each study depending upon the toxicity and potency of the test compound. A typical study design would involve dosing daily (M-F) with the test compound with recovery on the weekend. Throughout the study, tumor volumes and body weights are measured twice a week. At the end of the study the animals are euthanized and the tumors harvested and frozen for further analysis. Alternatively, tumors may be processed immediately for analysis, e.g. fixed in buffered-formalin, paraffin embedded, and sectioned for hematoxylin/eosin staining and further immunohistochemical analysis for desired oncology markers.

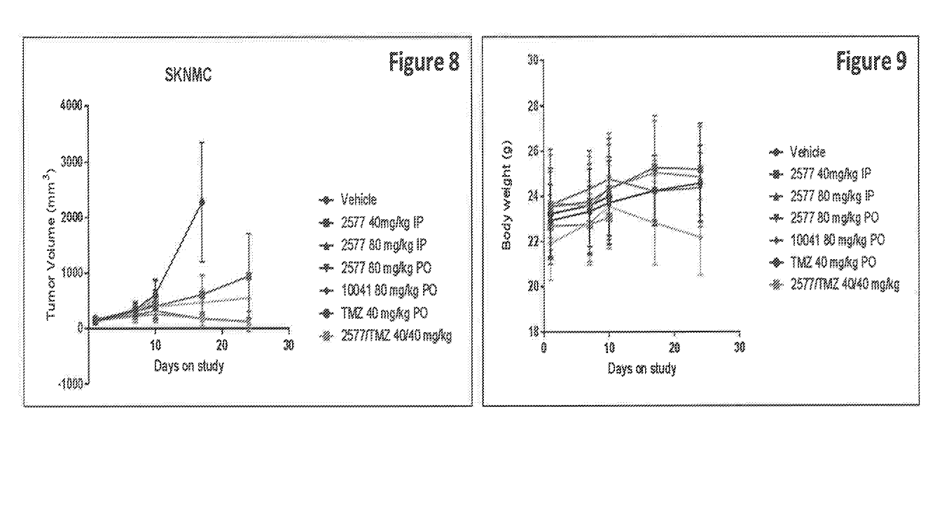

For example, compounds of the invention, or a pharmaceutically acceptable salt, solvate, polymorph, hydrate and the stereochemically isomeric form thereof, are expected to show such in vivo effects. The selected benzohydrazide compound (10041) was assessed for its antitumor properties. Its efficacy was demonstrated in mice SKNMC Ewing's Sarcoma model (FIGS. 8 and 9). These Figures show that 10041 compound was extremely effective.

Prophetic Pharmaceutical Composition Examples

“Active ingredient” as used throughout these examples relates to one or more of the compounds of the invention, or a pharmaceutically acceptable salt, solvate, polymorph, hydrate and the stereochemically isomeric form thereof. The following examples of the formulation of the compounds of the present invention in tablets, suspension, injectables and ointments are prophetic.

Typical examples of recipes for the formulation of the invention are as given below. Various other dosage forms can be applied herein such as a filled gelatin capsule, liquid emulsion/suspension, ointments, suppositories or chewable tablet form employing the disclosed compounds in desired dosage amounts in accordance with the present invention. Various conventional techniques for preparing suitable dosage forms can be used to prepare the prophetic pharmaceutical compositions, such as those disclosed herein and in standard reference texts, for example the British and US Pharmacopoeias, Remington's Pharmaceutical Sciences (Mack Publishing Co.) and Martindale The Extra Pharmacopoeia (London The Pharmaceutical Press).

The disclosure of this reference is hereby incorporated herein by reference.

A. Pharmaceutical Composition for Oral Administration

A tablet can be prepared as follows:

| Component | Amount | |
|---|---|---|
| Active ingredient | 10 to 500 | mg |
| Lactose | 100 | mg |
| Crystalline cellulose | 60 | mg |
| Magnesium stearate | 5 | |
| Starch (e.g. potato starch) | Amount necessary to yield total | |
| | weight indicated below | |
| Total (per capsule) | 1000 | mg |

Alternatively, about 100 mg of a disclosed compound, 50 mg of lactose (monohydrate), 50 mg of maize starch (native), 10 mg of polyvinylpyrrolidone (PVP 25) (e.g. from BASF, Ludwigshafen, Germany) and 2 mg of magnesium stearate are used per tablet. The mixture of active component, lactose and starch is granulated with a 5% solution (m/m) of the PVP in water. After drying, the granules are mixed with magnesium stearate for 5 min. This mixture is moulded using a customary tablet press (e.g. tablet format: diameter 8 mm, curvature radius 12 mm). The moulding force applied is typically about 15 kN.

Alternatively, a disclosed compound can be administered in a suspension formulated for oral use. For example, about 100-5000 mg of the desired disclosed compound, 1000 mg of ethanol (96%), 400 mg of xanthan gum, and 99 g of water are combined with stirring. A single dose of about 10-500 mg of the desired disclosed compound according can be provided by 10 ml of oral suspension.

In these Examples, active ingredient can be replaced with the same amount of any of the compounds according to the present invention, in particular by the same amount of any of the exemplified compounds. In some circumstances it may be desirable to use a capsule, e.g. a filled gelatin capsule, instead of a tablet form. The choice of tablet or capsule will depend, in part, upon physicochemical characteristics of the particular disclosed compound used.

Examples of alternative useful carriers for making oral preparations are lactose, sucrose, starch, talc, magnesium stearate, crystalline cellulose, methyl cellulose, hydroxypropyl cellulose, hydroxypropylmethyl cellulose, carboxymethyl cellulose, glycerin, sodium alginate, gum arabic, etc. These alternative carriers can be substituted for those given above as required for desired dissolution, absorption, and manufacturing characteristics.

The amount of a disclosed compound per tablet for use in a pharmaceutical composition for human use is determined from both toxicological and pharmacokinetic data obtained in suitable animal models, e.g. rat and at least one non-rodent species, and adjusted based upon human clinical trial data. For example, it could be appropriate that a disclosed compound is present at a level of about 10 to 1000 mg per tablet dosage unit.

B. Pharmaceutical Composition for Injectable Use

A parenteral composition can be prepared as follows:

| Component | Amount | |
|---|---|---|
| Active ingredient | 10 to 500 | mg |
| Sodium carbonate | 560 | mg* |
| Sodium hydroxide | 80 | mg* |
| Distilled, sterile water | Quantity sufficient to prepare | |
| | total volumen indicated below | |
| Total (per capsule) | 10 | ml per ampule |

*Amount adjusted as required to maintain physiological pH in the context of the amount of active ingredient, and form of active ingredient, e.g. a particular salt form of the active ingredient.

Alternatively, a pharmaceutical composition for intravenous injection can be used, with composition comprising about 100-5000 mg of a disclosed compound, 15 g polyethylenglycol 400 and 250 g water in saline with optionally up to about 15% Cremophor EL, and optionally up to 15% ethyl alcohol, and optionally up to 2 equivalents of a pharmaceutically suitable acid such as citric acid or hydrochloric acid are used. The preparation of such an injectable composition can be accomplished as follows: The disclosed compound and the polyethylenglycol 400 are dissolved in the water with stirring. The solution is sterile filtered (pore size 0.22 μm) and filled into heat sterilized infusion bottles under aseptic conditions. The infusion bottles are sealed with rubber seals.

In a further example, a pharmaceutical composition for intravenous injection can be used, with composition comprising about 10-500 mg of a disclosed compound, standard saline solution, optionally with up to 15% by weight of Cremophor EL, and optionally up to 15% by weight of ethyl alcohol, and optionally up to 2 equivalents of a pharmaceutically suitable acid such as citric acid or hydrochloric acid. Preparation can be accomplished as follows: a desired disclosed compound is dissolved in the saline solution with stirring. Optionally Cremophor EL, ethyl alcohol or acid are added. The solution is sterile filtered (pore size 0.22 m) and filled into heat sterilized infusion bottles under aseptic conditions. The infusion bottles are sealed with rubber seals.

In this Example, active ingredient can be replaced with the same amount of any of the compounds according to the present invention, in particular by the same amount of any of the exemplified compounds.

The amount of a disclosed compound per ampule for use in a pharmaceutical composition for human use is determined from both toxicological and pharmacokinetic data obtained in suitable animal models, e.g. rat and at least one non-rodent species, and adjusted based upon human clinical trial data. For example, it could be appropriate that a disclosed compound is present at a level of about 10 to 1000 mg per tablet dosage unit.

Carriers suitable for parenteral preparations are, for example, water, physiological saline solution, etc. which can be used with tris(hydroxymethyl)aminomethane, sodium carbonate, sodium hydroxide or the like serving as a solubilizer or pH adjusting agent. The parenteral preparations contain preferably 50 to 1000 mg of a disclosed compound per dosage unit.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula (I): 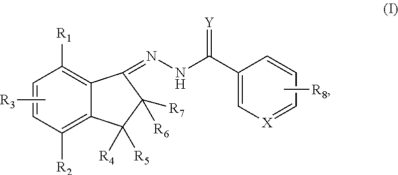
wherein
X is CH or N;
Y is O or S;
R₁, R₂ and R₃ are independently selected from the group consisting of hydrogen, OH, a C₁₋₆ alkyl, NH₂, a halogen, CF₃, OCF₃, O—(C₁₋₆ alkyl); and CN;
R₄, R₅, R₆ and R₇ are independently selected from the group consisting of hydrogen, a C₁₋₆ alkyl, and a halogen;
R₈ is 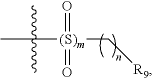
R₉ is selected from the group consisting of CH₃, NH₂, NCH₃, a C₁₋₆ alkyl, a C₁₋₆ cycloalkyl, a halogen-C₁₋₆ alkyl, a cycloalkyl, a C₁₋₆ heterocycloalkyl, aziridinyl, azetidinyl, pyrrolidinyl, piperidinyl, azepanyl, oxazolidinyl, imidazolidinyl, pyrazolidinyl, piperazinyl, oxazinanyl, morpholinyl, hexahydrophyrimidinyl, hexahydropyridazinyl and an optionally substituted moiety selected from the group consisting of: 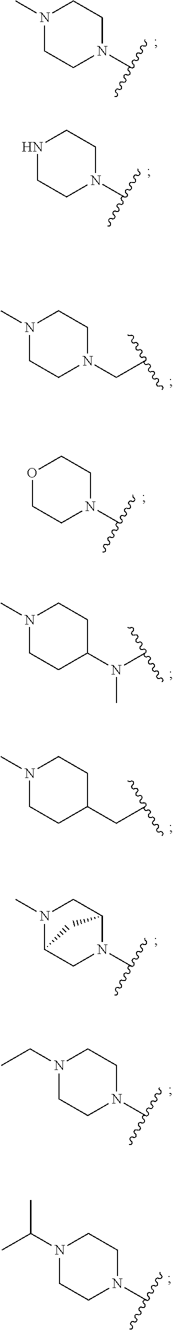 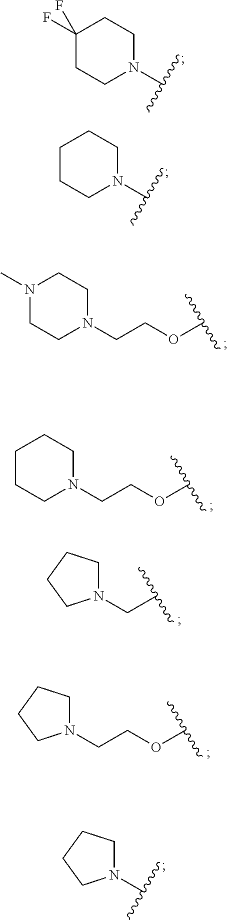 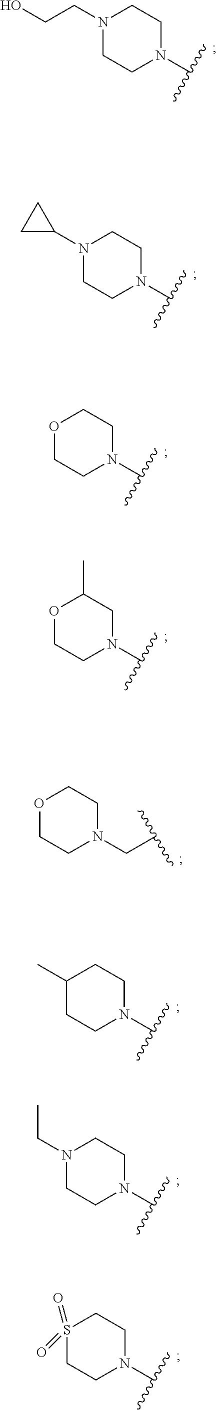 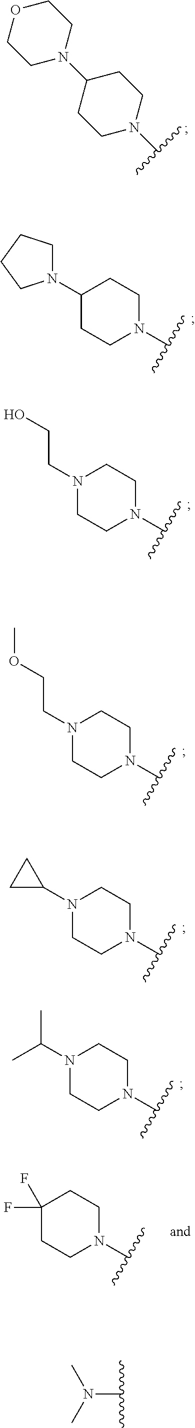
m is 0 or 1;
n is 0 or 1; with the proviso that when:
a) R₂ is a halogen; and
b) R₃ is H; and
c) m is 1; and
d) n is 0; then
R₁ cannot be OH.
or an isomer or a pharmaceutically acceptable salt thereof.

2. The compound of claim 1, wherein
X is CH and
Y is O.

3. The compound of claim 1, wherein
R₁ is selected from the group consisting of H, a halogen, an alkyl and OH;
R₂ is selected from the group consisting of H and a halogen;
R₃ is selected from the group consisting of H, OH and an alkyl;
R₄, R₅, R₆ and R₇ are H;
n is 0; and
R₉ is selected from the group consisting of:

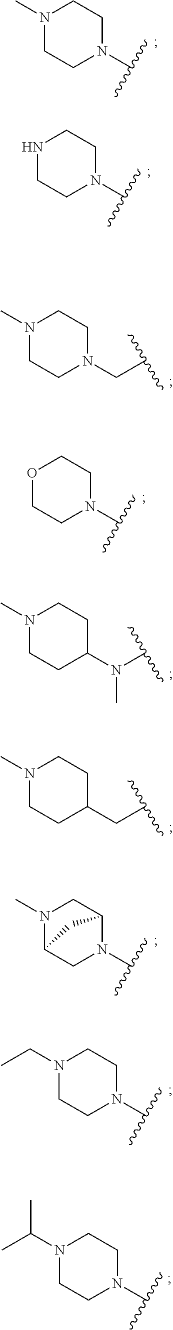

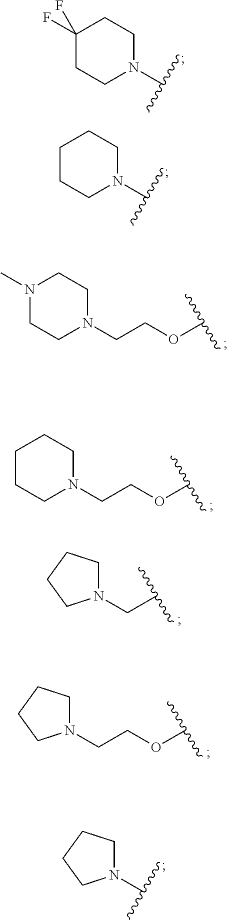

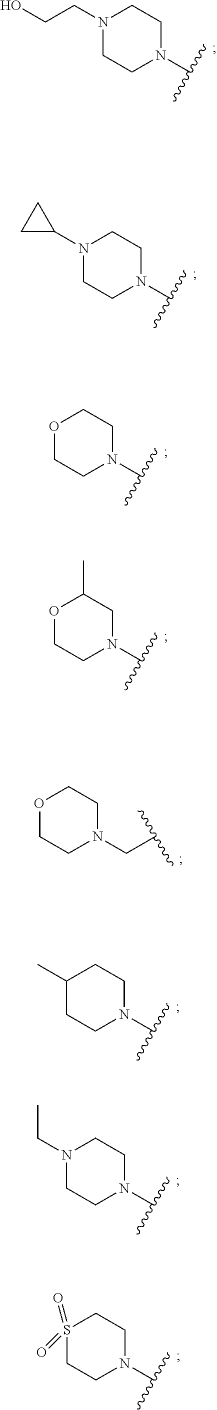

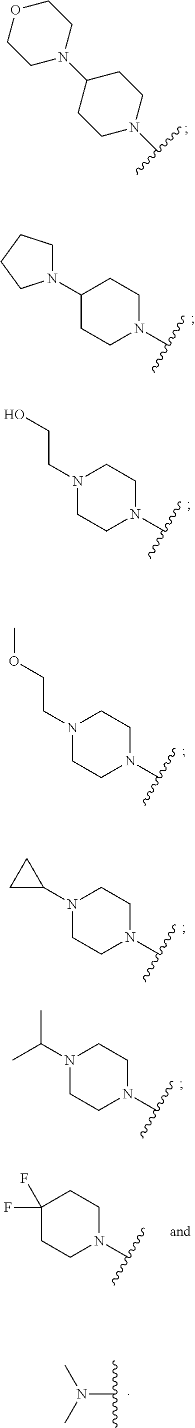

4. A compound selected from the group consisting of: 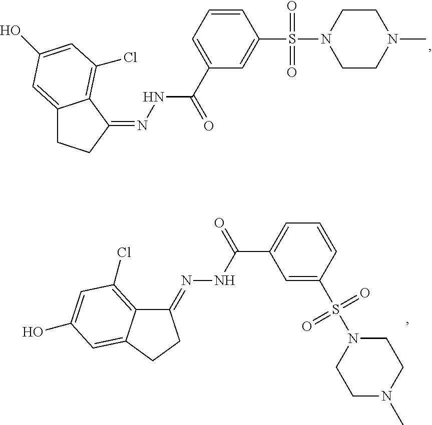 [SMILES: CN1CCN(S(=O)(=O)c2cccc(C(=O)N/N=C3/CCc4cc(O)cc(Cl)c43)c2)CC1 ; CN1CCN(S(=O)(=O)c2cccc(C(=O)N/N=C3\CCc4cc(O)cc(Cl)c43)c2)CC1] 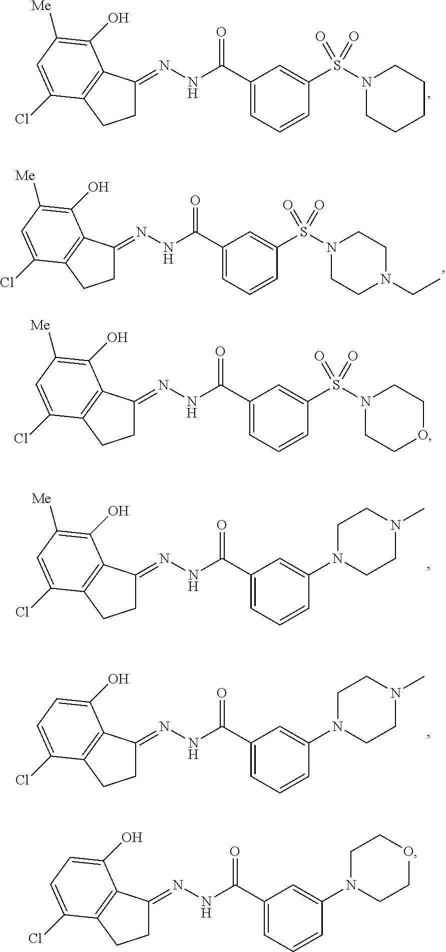 [SMILES: Cc1cc(Cl)c2c(c1O)/C(=N/NC(=O)c1cccc(S(=O)(=O)N3CCCCC3)c1)CC2 ; CCN1CCN(S(=O)(=O)c2cccc(C(=O)N/N=C3\CCc4c(Cl)cc(C)c(O)c43)c2)CC1 ; Cc1cc(Cl)c2c(c1O)/C(=N/NC(=O)c1cccc(S(=O)(=O)N3CCOCC3)c1)CC2 ; Cc1cc(Cl)c2c(c1O)/C(=N/NC(=O)c1cccc(N3CCN(C)CC3)c1)CC2 ; CN1CCN(c2cccc(C(=O)N/N=C3\CCc4c(Cl)ccc(O)c43)c2)CC1 ; O=C(N/N=C1\CCc2c(Cl)ccc(O)c21)c1cccc(N2CCOCC2)c1] 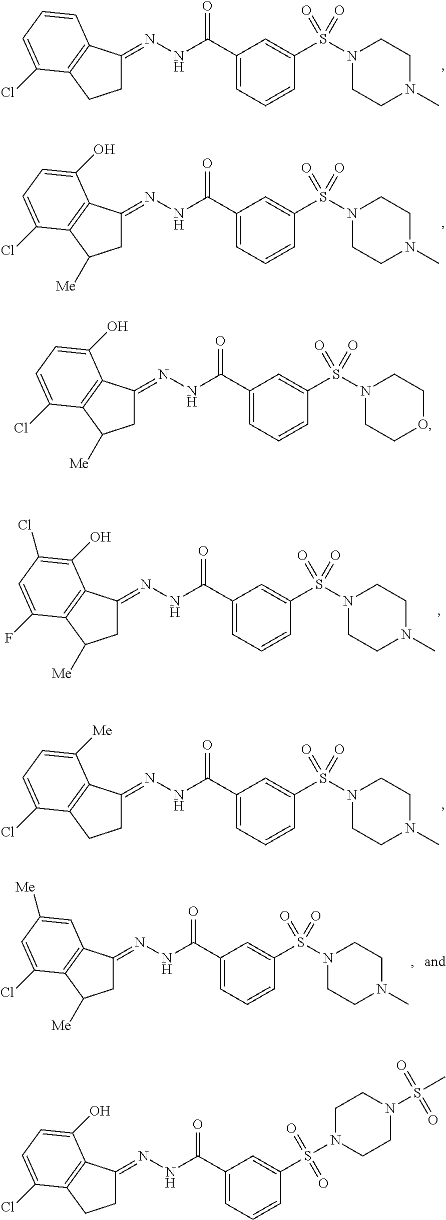
or an isomer or a pharmaceutically acceptable salt thereof.

5. A compound having a structure:

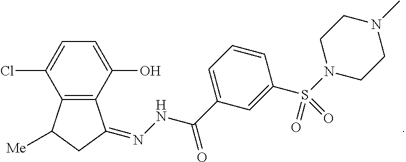 [SMILES: CC1C/C(=N/NC(=O)c2cccc(S(=O)(=O)N3CCN(C)CC3)c2)c2c(O)ccc(Cl)c21]

6. A compound having a structure:

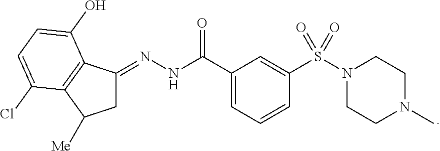 [SMILES: CC1C/C(=N\NC(=O)c2cccc(S(=O)(=O)N3CCN(C)CC3)c2)c2c(O)ccc(Cl)c21]

7. A compound having a structure:

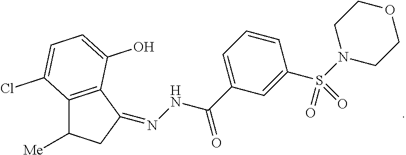 [SMILES: CC1C/C(=N/NC(=O)c2cccc(S(=O)(=O)N3CCOCC3)c2)c2c(O)ccc(Cl)c21]

8. A pharmaceutical composition comprising a therapeutically effective amount of a compound of claim 1 and a pharmaceutically acceptable carrier.

9. A method for the treatment of a disorder of uncontrolled cellular proliferation in a mammal, the method comprising the step of administering to the mammal an effective amount of a compound of claim 1.

10. A method for decreasing histone demethylase activity in a mammal, the method comprising the step of administering to the mammal an effective amount of a compound of claim 1.

11. A method for inhibiting lysine specific demethylase 1 (LSD1) activity in a mammal, the method comprising the step of administering to the mammal an effective amount of any of the compounds of the invention.

12. A method for inhibiting lysine specific demethylase 2 (LSD2) activity in a mammal, the method comprising the step of administering to the mammal an effective amount of a compound of compound having a structure represented by a formula (II): 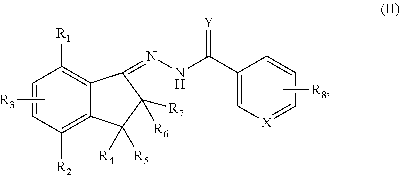
wherein
X is CH or N;
Y is O or S;
R₁, R₂ and R₃ are independently selected from the group consisting of hydrogen, OH, a C₁₋₆ alkyl, NH₂, a halogen, CF₃, OCF₃, O—(C₁₋₆ alkyl); and CN;
R₄, R₅, R₆ and R₇ are independently selected from the group consisting of hydrogen, a C₁₋₆ alkyl, and a halogen;
R₈ is 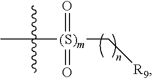
R₉ is selected from the group consisting of CH₃, NH₂, NCH₃, a C₁₋₆ alkyl, a C₁₋₆ cycloalkyl, a halogen-C₁₋₆ alkyl, a cycloalkyl, a C₁₋₆ heterocycloalkyl, aziridinyl, azetidinyl, pyrrolidinyl, piperidinyl, azepanyl, oxazolidinyl, imidazolidinyl, pyrazolidinyl, piperazinyl, oxazinanyl, morpholinyl, hexahydrophyrimidinyl, hexahydropyridazinyl and an optionally substituted moiety selected from the group consisting of: 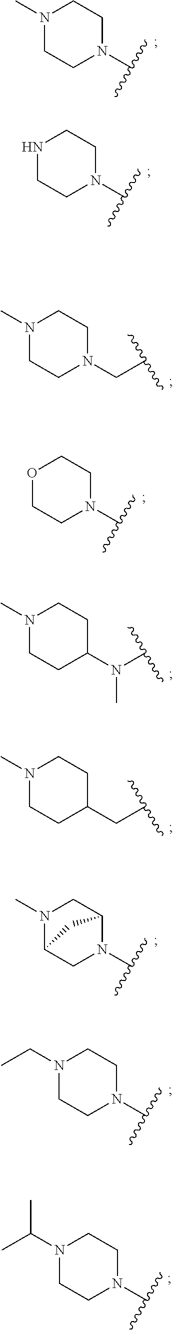 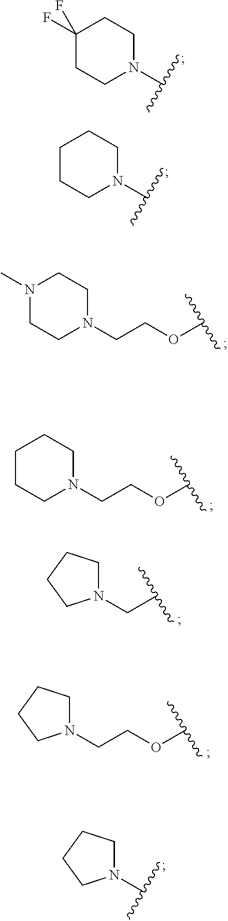 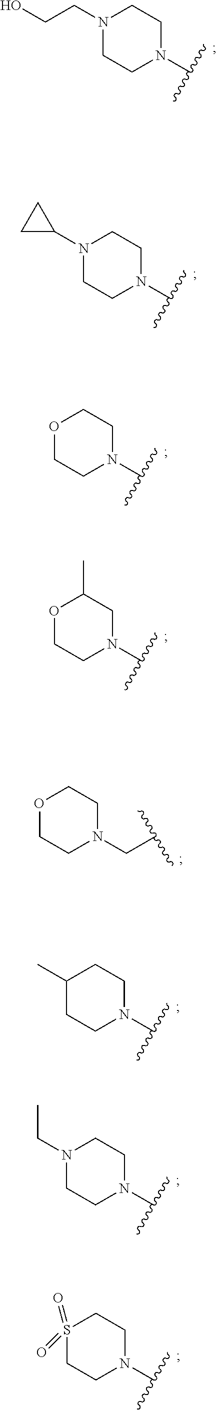 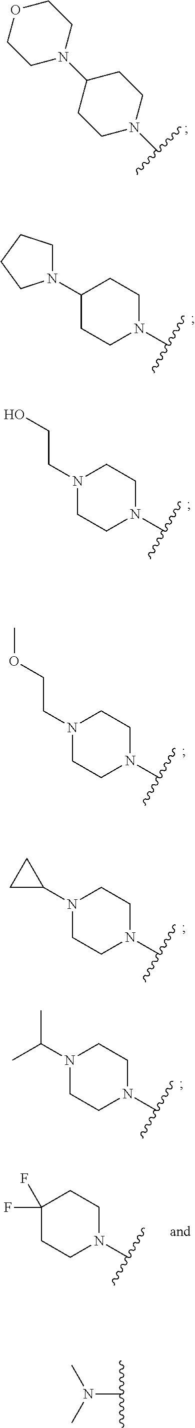
m is 0 or 1;
n is 0 or 1;
or an isomer or a pharmaceutically acceptable salt thereof.

13. The method of claim 12, wherein
X is CH and
Y is O.

14. The method of claim 12, wherein
R₁ is selected from the group consisting of H, a halogen, an alkyl and OH;
R₂ is selected from the group consisting of H and a halogen;
R₃ is selected from the group consisting of H, OH and an alkyl;
R₄, R₅, R₆ and R₇ are H;
n is 0; and
R₉ is selected from the group consisting of:

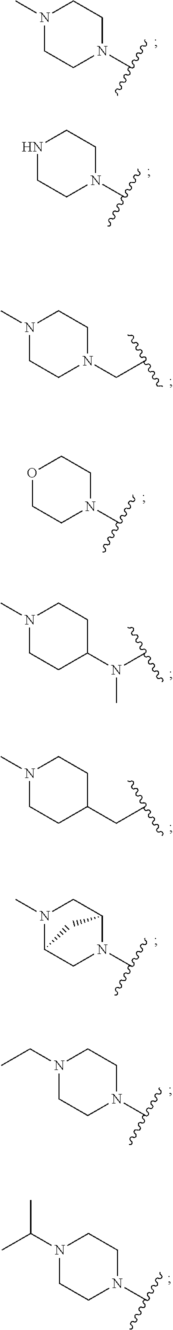

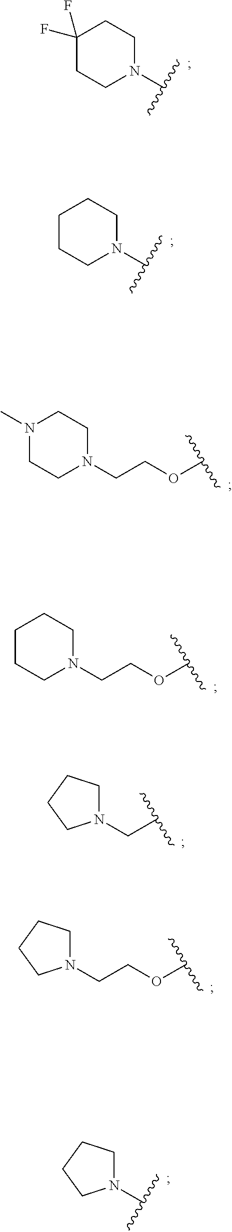

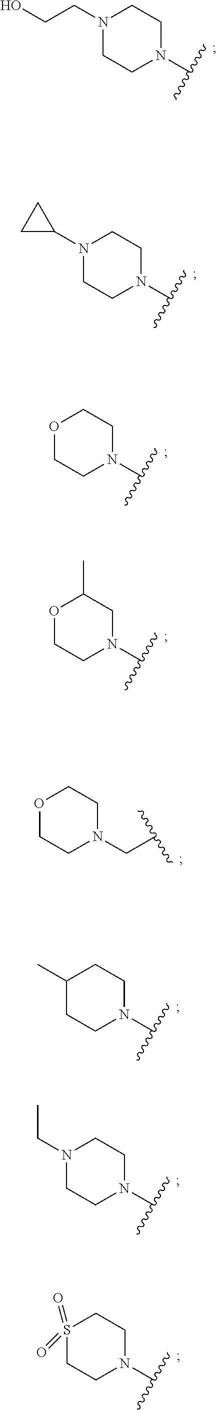

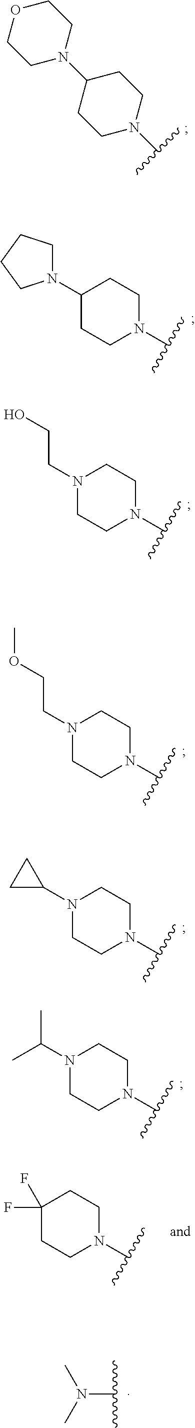

15. The method of claim 12, wherein the compound is selected from the group consisting of: 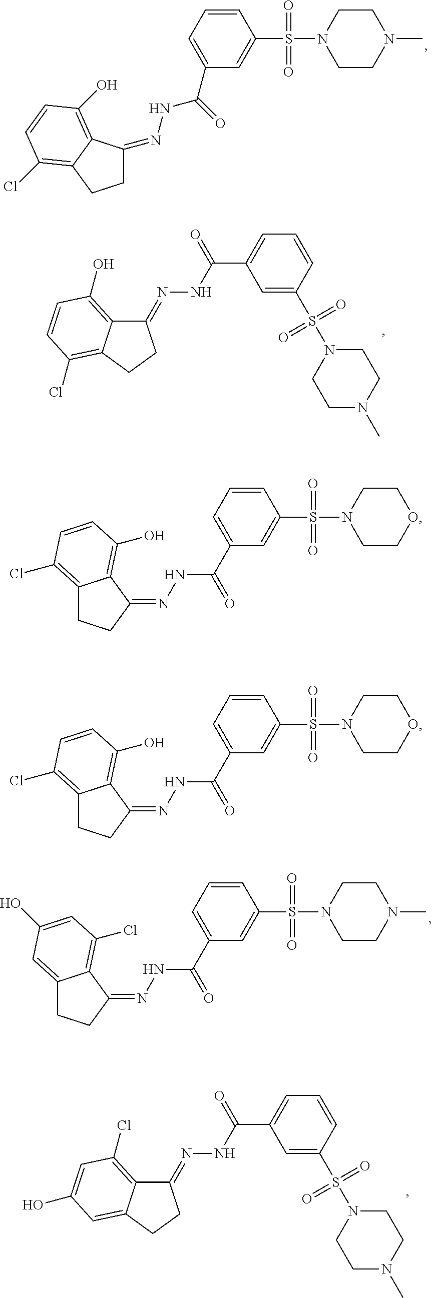 [SMILES: CN1CCN(S(=O)(=O)c2cccc(C(=O)N/N=C3/CCc4c(Cl)ccc(O)c43)c2)CC1 ; CN1CCN(S(=O)(=O)c2cccc(C(=O)N/N=C3\CCc4c(Cl)ccc(O)c43)c2)CC1 ; O=C(N/N=C1/CCc2c(Cl)ccc(O)c21)c1cccc(S(=O)(=O)N2CCOCC2)c1 ; O=C(N/N=C1/CCc2c(Cl)ccc(O)c21)c1cccc(S(=O)(=O)N2CCOCC2)c1 ; CN1CCN(S(=O)(=O)c2cccc(C(=O)N/N=C3/CCc4cc(O)cc(Cl)c43)c2)CC1 ; CN1CCN(S(=O)(=O)c2cccc(C(=O)N/N=C3\CCc4cc(O)cc(Cl)c43)c2)CC1] 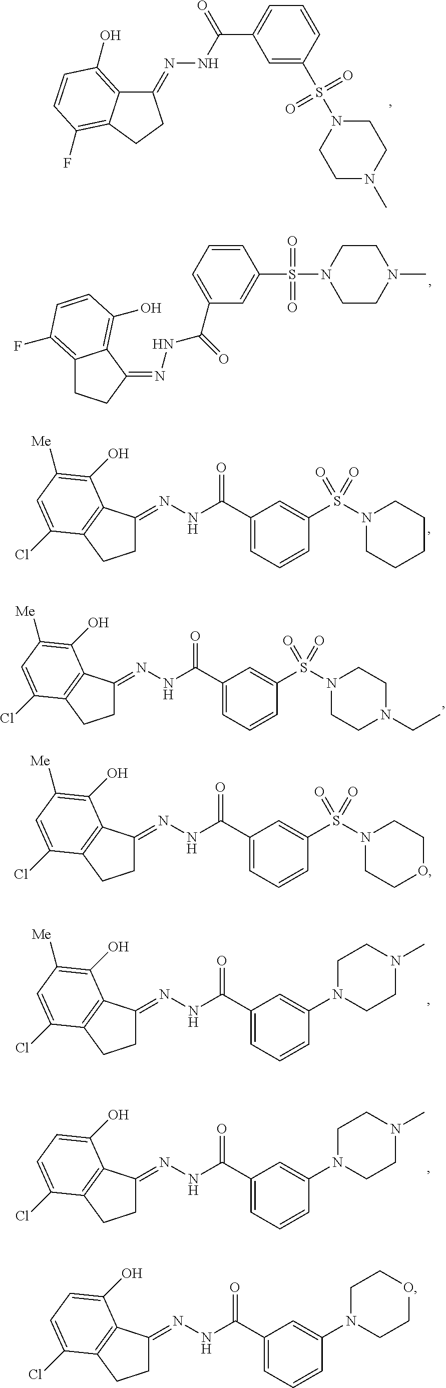 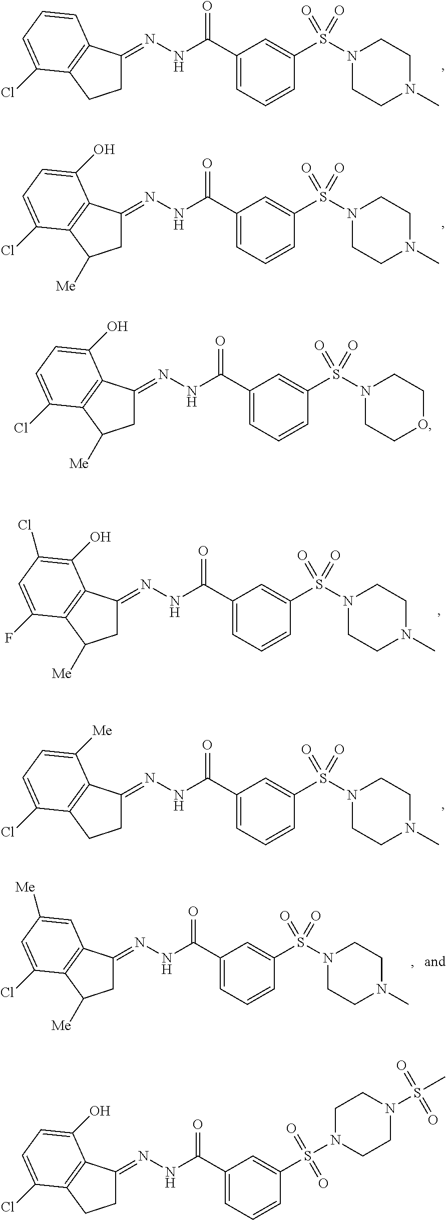
or an isomer or a pharmaceutically acceptable salt thereof.

16. The method of claim 12, wherein the compound has a following structure:

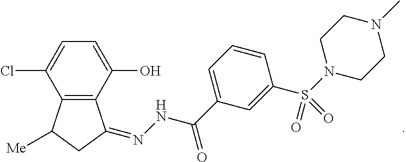 [SMILES: CC1C/C(=N/NC(=O)c2cccc(S(=O)(=O)N3CCN(C)CC3)c2)c2c(O)ccc(Cl)c21]

17. The method of claim 12, wherein the compound has a following structure:

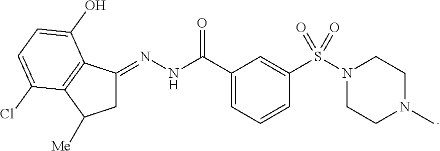 [SMILES: CC1C/C(=N\NC(=O)c2cccc(S(=O)(=O)N3CCN(C)CC3)c2)c2c(O)ccc(Cl)c21]

18. The method of claim 12, wherein the compound has a following structure: 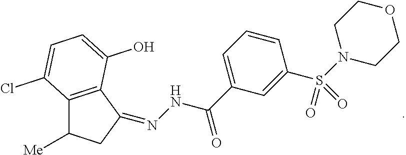 [SMILES: CC1C/C(=N/NC(=O)c2cccc(S(=O)(=O)N3CCOCC3)c2)c2c(O)ccc(Cl)c21]
